(19) United States
(12) Patent Application Publication
(10) Pub. No.: US 2003/0186281 A1
(43) Pub. Date: Oct. 2, 2003

(54) Modified tetracycline repressor protein compositions and methods of use

(76) Inventor: Wolfgang Hillen, Uttenreuth (DE)

Correspondence Address: PENNIE AND EDMONDS, 1155 AVENUE OF THE AMERICAS, NEW YORK, NY, 100362711

(21) Appl. No.: 10/326,671

(22) Filed: Dec. 20, 2002

Related U.S. Application Data
(60) Provisional application No. 60/343,278, filed on Dec. 21, 2001.

(51) Int. Cl. C12Q 1/68; G01N 33/554; G01N 33/569; C07H 21/04; C12N 9/06; C12N 1/21; C12P 21/02; C12N 15/74

(52) U.S. Cl. 435/6; 435/7.32; 435/69.1; 435/252.3; 435/252.31; 435/320.1; 536/23.2; 435/191

(57) ABSTRACT

The present invention relates to a system for regulating gene expression in prokaryotes using modified tetracycline repressor proteins. In particular, the present invention relates to modified tetracycline repressor proteins that exhibit a “reverse” phenotype in prokaryotic organisms, nucleic acids encoding these repressor proteins, methods for identifying and preparing these proteins, and methods for using these proteins for regulating gene expression in prokaryotic organisms, in drug screening assays and for identifying non-antibiotic compounds that are specific inducers of these modified repressor proteins.

This application claims the benefit of priority of U.S. provisional application serial No. 60/343,278, filed Dec. 21, 2001, which is incorporated herein by reference, in its entirety.

### 1. INTRODUCTION

The present invention relates to a system for regulating gene expression in prokaryotes using modified tetracycline repressor proteins. In particular, the present invention relates to modified tetracycline repressor proteins that exhibit a “reverse” phenotype in prokaryotic organisms, nucleic acids encoding these repressor proteins, methods for identifying and preparing these proteins, and methods for using these proteins for regulating gene expression in prokaryotic organisms, in drug screening assays and for identifying non-antibiotic compounds that are specific inducers of these modified repressor proteins.

### 2. BACKGROUND OF THE INVENTION

Increased resistance of pathogenic organisms to conventional antibiotics is a serious clinical problem confronting physicians and health care providers. Resistance to tetracycline (tet), a broad spectrum antibiotic that inhibits bacterial protein chain elongation, is one of the most common forms of antibiotic resistance encountered in bacteria, and at least three mechanisms have been described for conferring resistance: active efflux of tetracycline from the cell, protection of the ribosomal protein target and chemical degradation of the drug (for a general review of tetracycline resistance, see Hillen & Berens, (1994) Annu. Rev. Microbiol. 48:345-369).

The most abundant resistance mechanism against tetracycline in Gram-negative bacteria is active efflux of tetracycline from the cell, and resistance is often conferred to cells by tetracycline-resistance determinants that are encoded by mobile genetic elements. Certain mobile genetic elements, e.g., the transposon Tn10, contain two genes involved in resistance: a resistance gene, tetA, and a regulatory gene, tetR, which are transcribed from divergent promoters that are regulated by tetracycline. The resistance protein, TetA, is a tetracycline/metal-proton antiporter located in the cytoplasmic membrane and is responsible for efflux of tetracycline from the cell. The repressor protein, TetR, is a dimeric, DNA binding protein that regulates the expression of tetA and tetR at the level of transcription by binding in the absence of tetracycline to specific nucleotide sequences located within and overlapping the divergent promoter region (i.e., tandem tet operators O1 and O2; e.g., see Wissmann et al., (1991) Genetics 128:225-232). In the presence of tetracycline, TetR binds to intracellular tetracycline, which has a higher affinity for TetR than its target in the host. The binding of tetracycline results in an allosteric conformational change that greatly reduces the affinity of TetR for DNA thereby leaving the divergent promoters available for access by RNA polymerase, whereupon transcription of tetA and tetR is induced. Once tetracycline has been removed, the original conformation of TetR predominates and transcription of each promoter is repressed.

A number of different classes of Tet repressors and cognate operator sequences have been described, e.g., TetR(A), TetR(B), TetR(C), TetR(D), TetR(E), TetR(G), TetR(H), TetR(J), and TetR(Z). Individual Tet repressors are assigned to one of the above classes based upon nucleic acid hybridization, under stringent conditions, of the DNA encoding the associated efflux pump to that of the prototype for each class. In general, Tet repressors within each class exhibit at least 80% sequence identity (M. C. Roberts, 1996 FEMS. Microbiol. Reviews 19: 1-24), while the amino acid sequences between members of different classes of Tet repressors share a relatively high degree of homology (i.e., 40-60% across the length of the protein). In addition, Tet repressors have been subjected to extensive genetic and biochemical characterization, and a number of TetR variants haver been described, including modified tetracycline repressor fusion proteins that bind to tet operator DNA in eukaryotic cells only in the presence of tet (Gossen et al., (1995) Science 268:1766-1769). In the latter instance, these modified repressor proteins are used as fusion proteins containing an additional transactivator domain such that binding of the fusion protein via the DNA binding domain of TetR to a tet operator sequence engineered into a eukaryotic promoter results in transcriptional activation, not repression as described above for prokaryotic organisms. The presence of the additional transactivator domain as well as the dramatically different cellular environment between prokaryotic and eukaryotic organisms makes such fusion proteins undesirable for prokaryotic systems.

Notwithstanding the extensive amount of biochemical and genetic manipulation of tetracycline repressors over two decades, modified tetracycline repressors that exhibit a reverse phenotype in prokaryotic organisms (revTetR) have not yet been identified. Thus, there is a need to identify revTetR that are active in prokaryotic organisms, and that can provide a system for regulating gene expression in prokaryotic organisms.

### 3. SUMMARY OF THE INVENTION

A regulatory system that utilizes modified components of the Tet repressor/operator system to regulate gene expression in prokaryotic cells is provided. In particular, modified tetracycline repressor proteins that exhibit a “reverse” phenotype in prokaryotes, nucleic acids encoding these proteins, methods for identifying and preparing these proteins, and methods of use therefor in regulating gene expression in prokaryotic organisms, in drug screening assays, and in the identification of non-antibiotic molecules that are specific inducers of the instant revTetR repressors are provided.

In one embodiment, modified tetracycline repressor polypeptides that exhibit a “reverse” phenotype (revTetR) in prokaryotic organisms are provided. The revTetR repressors of the present invention bind to a tet operator DNA sequence in prokaryotes with a greater affinity (i.e., with a lower dissociation constant or K_{d} value) in the presence of tetracycline or tetracycline analog than in the absence of tetracycline or tetracycline analog. In one aspect, revTetR that exhibit the reverse phenotype in prokaryotes only at defined temperatures, e.g., at 28° C. or at 37° C., are provided.

In certain embodiments, the isolated nucleic acids comprise a nucleotide sequence encoding modified revTetR proteins that exhibit the reverse phenotype in prokaryotes only at particular “permissive” temperatures, e.g., at 28° C., while exhibiting essentially undetectable binding to a tet operator sequence at other “non-permissive” temperatures, e.g. 37° C. In still further embodiments, the isolated nucleic acids comprise a nucleotide sequence encoding modified revTetR proteins that exhibit the reverse phenotype in prokaryotes only at particular “permissive” temperatures, e.g., at 37° C., while exhibiting essentially undetectable binding to a tet operator sequence at other “non-permissive” temperatures, e.g. 28° C. In such embodiments, transcription in a prokaryote from a promoter operably associated with a tet operator is at least ten-fold greater at the permissive temperature than it is at the non-permissive temperature. In other such embodiments, transcription in a prokaryote from a promoter operably associated with a tet operator is at least twenty-fold or at least forty-fold greater at the permissive temperature than it is at the non-permissive temperature.

In one preferred embodiment, the modified tetracycline repressor is a chimeric revTetR that comprises the DNA binding domain of TetR(B) (e.g., amino acid residues 1-50 of SEQ ID NO. 32) and the tetracycline binding pocket of TetR(D), (e.g., amino acid residues 51-208 of SEQ ID NO. 32), i.e., a TetR(BD), and further comprises at least one amino acid substitution at position 96 or 99, or substitutions at positions 96, 103 and 114; positions 96, 157 and 200; positions 96 and 159; positions 160, 178, 196; positions 59, 95 and 100; positions 96 and 188; positions 96 and 205; positions 96 and 110; positions 99 and 194; positions 99 and 158; positions 70, 91 and 99; positions 71, 95 and 127; positions 59, 98, 101 and 192, of SEQ ID NO: 32.

Presently preferred amino acid substitutions that confer a reverse phenotype in prokaryotes include, but are not limited to, Asn at position 59, Val at positions 70 and 71; Gln at position 91; Glu and Gly at position 95; Arg and Glu at position 96; Arg at position 98; Glu at position 99; Ala at position 100; His at position 101; Ser at position 103; Phe at position 110; Val at position 114; Arg at position 127; Asn at position 157; Cys at position 158; Leu at position 159; Gln at position 188; Gly at position 192; Val at position 194; Trp at position 196; His at position 200; and Ser at position 205 of SEQ ID NO: 32. In more preferred embodiments, the revTetR comprises an amino acid sequence selected from any of one of the sequences set forth in SEQ ID NOS. 2, 4, 6, 8, 10, 12, 14, 16, 18, 20, 22, 24, 26, 28, and 30.

Additional examples of amino acid substitutions within the amino acid sequence of SEQ ID NO: 32 that confer a reverse phenotype on the encoded tetracycline repressor protein are provided. Accordingly, revTetR proteins of the present invention also include those comprising an amino acid sequence selected from the group consisting of SEQ ID NOS.: 71 to 264. The nucleotide sequences comprising the preferred nucleotide substitutions in these examples are provided in SEQ ID NOS.: 265 to 458.

In still another embodiment, revTetR comprising at least 6, 8, 10, 15, 18, 20, 25, 30, 35, 40, 45, 50 contiguous amino acids or more that contain at least one amino acid substitution that confers a reverse phenotype in prokaryotes are provided. Presently preferred peptides are those comprising at least one mutation conferring a reverse phenotype located within all or a portion of amino acid positions 90 to 105, 95 to 103; 110 to 127; 150 to 159; and 160 to 205 of SEQ ID NO: 32. Additional preferred peptides containing one or more amino acid substitutions that confer a reverse phenotype in prokaryotes include those made in a segment spanning amino acid positions 13-25, 14-24, and 17-23. In specific aspects of this embodiment, the revTetR protein comprises an amino acid substitution at a position selected from the group consisting of positions number 18, 22, 20, 23, and 17 of SEQ ID NO: 32, selected from the group consisting of positions 18, 20, and 22 of SEQ ID NO: 32, and more particularly, or at position 18 of SEQ ID NO: 32. Other preferred peptides comprising one or more amino acid substitutions that confer a reverse phenotype in prokaryotes include those made in a segment spanning amino acid positions 53-61 of SEQ ID NO: 32. In specific aspects of this embodiment, the revTetR protein comprises an amino acid substitution at a position selected from the group consisting of positions 59, 56, 53, 61, and 60 of SEQ ID NO: 32, and more particularly, selected from the group consisting of positions 59 and 56 of SEQ ID NO.; 32. Other preferred peptides comprisng one or more amino acid substitutions those made in a segmentt confer a reverse phenotype in prokaryotes include that spanning amino acid positions 95-99 of SEQ ID NO: 32. In a specific aspect of this embodiment, the revTetR comprises an amino acid substitution at a position selected from the group consisting of position 99 and 96 of SEQ ID NO: 32.

In other embodiments of the present invention, the specific amino acid substitutions identified as described herein with TetR(BD) chimeras, may also, in turn, be substituted by similar, functionally equivalent amino acids, as described infra, to provide additional revTetR repressors that are within the scope of the invention. Moreover, in certain embodiments, a revTetR repressor protein of the present invention can be constructed from any TetR repressor protein, in particular, the TetR protein of of the TetR(A), TetR(B), TetR(C), TetR(D), TetR(E), TetR(G), TetR(H), TetR(J), and TetR(Z) classes, by substituting, at the position corresponding to that identified in the TetR(BD) chimera depicted in SEQ ID NO: 32, either the exact amino acid identified in the revTet(BD) chimeras depicted in SEQ ID NO: 2, 4, 6, 8, 10, 12, 14, 16, 18, 20, 22, 24, 26, 28, 30, and 71-264, or the functional equivalent of that amino acid.

The amino acid substitutions of the present invention and their functional equivalents can be introduced into TetR proteins of each of the nine classes of TetR proteins, to provide novel revTet repressor proteins. The position of each of the amino acid substitutions disclosed above is numbered according to the amino acid sequence of the TetR(BD) chimeric protein of SEQ ID NO: 32. As would be apparent to one of ordinary skill, the corresponding amino acid to be substituted in another TetR protein such as, but not limited to those;members of the TetR(A), TetR(B), TetR(C), TetR(D), TetR(E), TetR(G), TetR(H), TetR(J), and TetR(Z) classes of TetR repressor proteins to provide a revTetR protein, is readily identified using methods and tools well known in the art. For example, the amino acid sequence of a subject TetR repressor is readily compared with that provided by SEQ ID NO: 32 using software publically available from the National Center for Biotechnology Information and the National Library of Medicine at http://www.ncbi.nlm.nih.gov/BLAST. (For a description of this software, see Tatusova et al. (1999) FEMS Microbiol Lett 177(1): 187-88).

For example, comparisons have been carried out for each representative TetR(A), TetR(B), TetR(C), TetR(D), TetR(E), TetR(G), TetR(H), TetR(J), and TetR(Z) protein disclosed above, to provide the position and nature of the amino acid corresponding to each of the substitutions disclosed herein, for each representative class member. The results of such comparisons are summarized in Table 1, where TetR(BD) is SEQ ID NO: 32, TetR(A) is SEQ ID NO: 34, TetR(B) is SEQ ID NO: 36, TetR(C) is SEQ ID NO: 38, TetR(D) is SEQ ID NO: 40, TetR(E) is SEQ ID NO: 42, TetR(G) is SEQ ID NO: 44, TetR(H) is SEQ ID NO: 46, TetR(J) is SEQ ID NO: 48, and TetR(Z) is SEQ ID NO: 50. The first column of Table 1 provides the wild type amino acid residue, the amino acid position number, and the substituted amino acid residue found at that position in the revTet(BD) mutants disclosed above. The corresponding amino acid position and wild type amino acid residue found at that position for each representative member of TetR A, B, C, D, E, G, H, J, and Z are provided in the remaining nine columns of Table 1.

In another aspect of the invention, isolated nucleic acids comprising nucleotide sequences encoding modified tetracycline repressor proteins that exhibit a “reverse” phenotype (revTetR) in prokaryotic cells are provided. In one embodiment, the isolated nucleic acids comprise a nucleotide sequence encoding modified revTetR proteins that bind to a tet operator DNA sequence in prokaryotes with a greater affinity (i.e., with a lower dissociation constant or K_{d} value) in the presence of tetracycline or tetracycline analog than in the absence of tetracycline or tetracycline analog. In other embodiments, the isolated nucleic acids comprise a nucleotide sequence encoding modified revTetR proteins that exhibit the reverse phenotype in prokaryotes only at particular temperatures, e.g., exhibit the reverse phenotype only at 28° C. or 37° C., but not both.

In preferred embodiments, the isolated nucleic acid molecules encode a chimeric revTetR repressor composed of the DNA binding domain of TetR(B) (e.g., amino acid residues 1-50 of SEQ ID NO. 32) and the tetracycline binding pocket of TetR(D), (e.g., amino acid residues 51-208 of SEQ ID NO. 32) and further comprises at least one mutation conferring a reverse phenotype in a prokaryotic organism.

In other embodiments, the isolated nucleic acids comprise a nucleotide sequence that encodes any of the amino acid sequences set forth in SEQ ID NOS. 2, 4, 6, 8, 10, 12, 14, 16, 18, 20, 22, 24, 26, 28, 30, and 71-264. In further embodiments, the isolated nucleic acids comprise the sequence of nucleotides selected from the group consisting of SEQ ID NOS. 1, 3, 5, 7, 9, 11, 13, 15, 17, 19, 21, 23, 25, 27, 29, and 265-458.

In still further embodiments, the isolated nucleic acid molecules encode a revTetR comprising a sequence of nucleotides including at least one revTetR mutation, and preferably having at least 35%, 40%, 50%, 60%, 70%, 75%, 80%, 85%, 90%, 95%, 98% or 99% nucleotide sequence identity, more preferably at least 90%, 95%, 98% or 99% sequence identity, to any of the nucleotide sequences set forth in SEQ ID NOS. 1, 3, 5, 7, 9, 11, 13, 15, 17, 19, 21, 23, 25, 27, 29, and 265-458.

In yet another embodiment, the isolated nucleic acid molecules comprise a sequence of nucleotides which comprises at least one revTetR mutation and hybridizes under moderate stringency conditions to the entire length of any of the nucleotide sequences set forth in SEQ ID NOS. 1, 3, 5, 7, 9, 11, 13, 15, 17, 19, 21, 23, 25, 27, 29, and 265-458. In still yet another embodiment, the isolated nucleic acid molecules comprise a sequence of nucleotides which comprises one or more revTetR mutation(s), and hybridizes under high stringency conditions to the entire length of any of the nucleotide sequences set forth in SEQ ID NOS. 1, 3, 5, 7, 9, 11, 13, 15, 17, 19, 21, 23, 25, 27, 29, and 265-458.

Isolated nucleic acids comprising a full-length complement of the nucleotide sequences any of these nucleic acids are also provided.

Isolated nucleic acid fragments of a revTetR comprising at least 10, 15, 20, 25, 30, 35, 40, 45 or 50 contiguous nucleotides comprising at least one mutation that confers a reverse phenotype in prokaryotes, or the complement thereof, are also provided. Particularly preferred nucleic acid fragments are those containing at least one mutation conferring a reverse phenotype in prokaryotic organisms located within nucleotide positions 210-216, 285 to 309, 330-381, 450-477, or 480 to 605 of SEQ ID NO. 31. Additional preferred nucleic acid fragments are those containing at least one mutation conferring a reverse phenotype in prokaryotic organisms within nucleotide positions 37-75, 40-72, 49-69, 157-183, and 283-297 of SEQ ID NO: 31.

In other embodiments, isolated nucleic acids comprising the coding region of a revTetR of the present invention operably linked to a nucleotide sequence containing a heterologous promoter are provided. In further embodiments, a vector or plasmid comprising nucleotide sequences encoding a revTetR of the present invention are provided.

In other embodiments, prokaryotic organisms comprising the isolated nucleic acids encoding a revTetR of the present invention are provided. Presently preferred prokaryotic organisms include, but are not limited to Bacillus anthracis, Bacteriodes fragilis, Bordetella pertussis, Burkholderia cepacia, Camplyobacter jejuni, Chlamydia pneumoniae, Chlamydia trachomatus, Clostridium botulinum, Clostridum tetani, Clostridium perfringens, Clostridium difficile, Corynebacterium diptheriae, Enterobacter cloacae, Enterococcus faecalis, Escherichia coli, Haemophilus influenzae, Helicobacter pylori, Klebsiella pneumoniae, Listeria monocytogenes, Moraxella catarrhalis, Mycobacterium leprae, Mycobacterium tuberculosis, Neisseria gonorrhoeae, Nesseria meningitidis, Nocardia asteroides, Proteus vulgaris, Pseudomonas aeruginosa, Salmonella typhi, Salmonella typhimurium, Shigella boydii, Shigella dysenteriae, Shigella flexneri, Shigella sonnei, Staphylococcus aureus, Staphylococcus epidermidis, Streptococcus mutans, Streptococcus pneumoniae, Treptonema pallidum, Vibrio cholerae, Vibrio parahemolyticus, and Yersina pestis.

In yet another embodiment, antibodies to modified tetracycline repressor that specifically recognize a revTetR, but not wild type TetR, are provided. The antibodies may be polyclonal or monoclonal antibodies, and are more preferably monoclonal antibodies that are specific for the conformation of the resulting revTetR or specific against the epitopes comprising the substitutions that confer the reverse phenotype. Preferred antibodies of the present invention have binding affinities including those with a dissociation constant or K_{d} less than 5×10⁻⁶M, 10⁻⁶M, 5×10⁻⁷M, 10⁻⁷M, 5×10⁻⁸M, 10⁻⁸M, 5×10 ⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, or 10⁻¹⁵M.

In another embodiment, methods for preparing recombinant, modified tetracycline repressors that exhibit a reverse phenotype in prokaryotes are provided. In one aspect, the method comprises introducing into a prokaryotic organism an expressible nucleic acid comprising a nucleotide sequence encoding a modified tetracycline repressor that exhibits a reverse phenotype in the prokaryotic organism, expressing the modified tetracycline repressor protein in the organism, and purifying the expressed modified tetracycline repressor. In a preferred embodiment, the nucleotide sequence encoding the modified tetracycline repressor is selected from nucleotide sequence encoding any of the amino acid sequences of SEQ ID NOS. 2, 4, 6, 8, 10, 12, 14, 16, 18, 20, 22, 24, 26, 28, 30, and 71-264.

In another embodiment, methods for identifying modified tetracycline repressors that exhibit a reverse phenotype in prokaryotes are provided. The methods comprise introducing into prokaryotic organisms a collection of nucleic acids each comprising a reporter gene operatively linked to a promoter regulated by a tetracycline operator, and an expressible nucleic acid encoding a modified tetracycline repressor containing at least one, preferably different, amino acid substitutions relative to a wild type tetracycline repressor that binds the tetracycline operator in the absence of tetracycline or tetracycline analog; culturing the prokaryotic organism in the presence or absence of tetracycline or tetracycline analog, and under conditions such that the modified tetracycline repressor is expressed; comparing and identifying the prokaryotic organism that express the reporter gene at a higher level in the absence than in the presence of the tetracycline or tetracycline analog.

The modified tetracycline-regulated repressor proteins of the present invention are useful for regulating expression, in a highly controlled manner, of a gene linked to one or more tet operator sequences in prokaryotes. Methods for using the regulatory system for regulating expression of a tet operator-linked gene in a prokaryotic organism are provided. In one embodiment, the method comprises introducing into an organism a target gene of interest which is under the control of at least one tet operator and an expressible nucleotide sequence encoding a revTetR, and contacting the organism with a concentration of tetracycline or tetracycline analog sufficient to alter the level of transcription of the target gene. The methods of the invention also allow for the regulation of expression of an endogenous gene which has been operatively linked to one or more tet operator sequence(s) that binds the revTet of the invention. In a preferred embodiment, the nucleotide sequence encoding the revTetR repressor is selected from nucleotide sequence encoding any of the amino acid sequences of SEQ ID NOS. 2, 4, 6, 8, 10, 12, 14, 16, 18, 20, 22, 24, 26, 28, 30, and 71-264. Alternatively, the tet operator-linked gene can be an exogenous gene which has been introduced into the cells.

In another embodiment, methods for identifying genes or gene products essential for proliferation or pathogenicity of a prokaryotic organism are provided. In one embodiment, the method comprises introducing into the prokaryotic organism an expressible nucleic acid encoding a putatively essential gene for proliferation or pathogenicity under the control of a promoter and at least one tet operator, and an expression vector comprising a nucleotide sequence encoding a modified tetracycline repressor, wherein said modified tetracycline repressor binds to a tetracycline operator sequence in a prokaryotic organism with a greater affinity in the presence of tetracycline or a tetracycline analog than in the absence of tetracycline or a tetracycline analog. The prokaryotic organism is cultured under conditions such that the modified tetracycline repressor is expressed, and in the presence of tetracycline or tetracycline analog at a concentration sufficient to repress expression of the putative essential gene. In a preferred aspect of this embodiment, the concentration of tetracycline or tetracycline analog sufficient to repress expression of the putative essential gene is a sub-inhibitory concentration. The viability or pathogenicity of the prokaryotic organism is determined, whereby a lack or decrease in viability or pathogenicity in the presence of the antibiotic indicate that the gene is essential or required for pathogenesis.

In yet another embodiment, methods for identifying compounds that inhibit an essential gene or gene product are provided. The method comprises introducing into the prokaryotic organism a nucleic acid comprising a nucleotide sequence encoding an essential gene under the control of at least one tet operator, and an expressible nucleic acid encoding a modified tetracycline repressor, wherein said modified tetracycline repressor binds to a tetracycline operator sequence in the prokaryotic organism with a greater affinity in the presence of tetracycline or a tetracycline analog than in the absence of tetracycline or a tetracycline analog; culturing the prokaryotic organism under conditions such that the modified tetracycline repressor is expressed and in the presence of tetracycline or tetracycline analog at a concentration sufficient to repress expression of the essential gene; contacting the prokaryotic organism with a test compound; and determining the effect of the test compound compared to control cell not cultured in tetracycline or tetracycline analog. In a further aspect of this embodiment, the control cell comprises an expressible nucleic acid encoding the modified tetracycline repressor and is cultured in the presence of the tetracycline or tetracyline analog, but the essential gene of the control cell is not under the control of a tet operator.

In yet another embodiment, methods for identifying non-antibiotic compounds that mimic tetracycline or its analog and can modulate the binding affinity of the modified tetracycline repressor to a tetracycline operator, are provided. Preferably, the non-antibiotic compounds specifically interact with revTetR to produce the reverse phenotype in prokaryotes. The method comprises introducing into the prokaryotic organism a nucleic acid comprising a reporter gene operatively linked to a promoter regulated by a tetracycline operator, and an expression vector comprising a nucleotide sequence encoding the modified tetracycline repressor; culturing the prokaryotic organism in the presence or absence of the non-antibiotic compound, and under conditions such that the modified tetracycline repressor is expressed; and identifying the non-antibiotic compound that modulates expression of the reporter gene product.

In a further embodiment, methods for in vivo antibiotic screening are provided. In this embodiment, a prokaryotic organism comprising a target gene essential for proliferation or pathogenicity is placed under the control of a promoter and at least one tet operator, and an expressible nucleotide sequence encoding a revTetR. Hence, expression of the revTetR gene in the recombinant prokaryotic organism regulates the level of expression of the target gene product required for growth and/or pathogenicity. Such a recombinant organism is used to infect a suitable animal model of a disease caused by the prokaryotic organism, e.g. a mouse model of an infectious disease, and the level of expression of the essential and/or virulence gene or genes is modulated by the level of tetracycline or its analog provided to the test mouse, e.g., in its drinking water. The beneficial effect(s) of the test compound on the infected animal is compared with control animals not provided with the antibiotic. In this manner, the virulence and/or growth rate of the pathogen may be regulated, providing a test system of variable sensitivity in an animal model. The sensitivity of the system can be adjusted by the amount of tetracycline in the system. That is, minimal expression of the regulated target gene product will provide a system capable of detecting low levels of active compound, as well as, higher levels of less-active compound that may serve as a lead structure for further development. Alternatively, high level expression of the regulated gene provides a less sensitive system in which only the most active compounds will be detected.

### 4. BRIEF DESCRIPTION OF THE DRAWINGS

FIG. 1: FIG. 1 illustrates the alignment of the primary amino acid sequences of the following TetR repressor proteins: TetR(A) (SEQ ID NO: 34); TetR(B) (SEQ ID NO: 36); TetR(C) (SEQ ID NO: 38); TetR(D) (SEQ ID NO: 40); TetR(E) (SEQ ID NO: 42); TetR(G) (SEQ ID NO: 70), which represents a combination of three Genbank Accession Files: AF133139, AF133140, and S52438; TetR(H) (SEQ ID NO: 46); TetR(30) (SEQ ID NO: 69); and TetR(Z) (SEQ ID NO: 50).

FIG. 2: FIG. 2 shows the relative activity of the modified TetR repressors that exhibit a reverse phenotype in prokaryotes. The relative activity of revTetR repressors was determined at 28° C. and 37° C. for each clone by measuring β-galactosidase activity of a tetracycline-regulated promoter in transformed Escherichia coli in the presence and absence of the tetracycline analog, anhydrotetracycline (atc). The relative β-galactosidase activity was measured in standard Miller units and is presented as the percent of maximal expression as measured in the absence of Tet repressor. The absolute levels of repressed and non-repressed transcription vary but each mutant demonstrates the reverse phenotype compared to wild type. With respect to each mutant, as well as the wild-type controls, FIG. 2 provides two horizontal bars; the upper horizontal bar represents the level of β-galactosidase activity in the absence of anhydrotetracycline (−atc) while the lower horizontal bar represents the level of β-galactosidase activity in the presence of anhydrotetracycline (+atc).

FIG. 3: FIG. 3 illustrates the time-dependent induction of tet-regulated transcription by revTetR repressors upon removal of the tetracycline analog, anhydrotetracycline (atc).

### 5. DETAILED DESCRIPTION OF THE INVENTION

5.1. Definitions

Unless defined otherwise, all technical and scientific terms used herein have the same meaning as is commonly understood by one of skill in the art to which this invention belongs. All patents and publications referred to herein are, unless noted otherwise, incorporated by reference in their entirety.

As used herein, “nucleotide sequence” refers to a heteropolymer of nucleotides, including but not limited to ribonucleotides and deoxyribonucleotides, or the sequence of these nucleotides. “Nucleic acid” and “polynucleotide” are also used interchangeably herein to refer to a heteropolymer of nucleotides, which may be unmodified or modified DNA or RNA. For example, polynucleotides can be single-stranded or double-stranded DNA, DNA that is a mixture of single-stranded and double-stranded regions, hybrid molecules comprising DNA and RNA with a mixture of single-stranded and double-stranded regions. In addition, the polynucleotide can be composed of triple-stranded regions comprising DNA, RNA, or both. A polynucleotide can also contain one or more modified bases, or DNA or RNA backbones modified for nuclease resistance or other reasons. Generally, nucleic acid segments provided by this invention can be assembled from fragments of the genome and short oligonucleotides, or from a series of oligonucleotides, or from individual nucleotides, to provide a synthetic nucleic acid.

As used herein, a “probe”, “primer”, or “fragment” is single-stranded DNA or RNA that has a sequence of nucleotides that includes at least 10 contiguous bases that are the same as (or the complement of) any 14 bases set forth in any of SEQ ID NOS. 1, 3, 5, 7, 9, 11, 13, 15, 17, 19, 21, 23, 25, 27, 29, and 265-458. Preferred regions from which to construct probes and primers include 5′ and/or 3′ coding sequences, sequences predicted to confer the reverse phenotype in prokaryotic organisms. Particularly preferred nucleic acid fragments are those containing at least one mutation conferring a reverse phenotype in prokaryotic organisms in the regions comprising nucleotides 210-216, 285 to 309, 330-381, 450-477, or 480 to 605 of SEQ ID NO. 31. Additional preferred nucleic acid fragments are those containing at least one mutation conferring a reverse phenotype in prokaryotic organisms in the regions comprising nucleotides 37-75, 40-72, 49-69, 157-183, and 283-297.

As used herein, “polypeptide” refers to the molecule formed by joining amino acids to each other by peptide bonds, and may contain amino acids other than the twenty commonly used gene-encoded amino acids. The term “active polypeptide” refers to those forms of the polypeptide which retain the biologic and/or immunologic activities of any naturally occurring polypeptide. The term “naturally occurring polypeptide” refers to polypeptides produced by cells that have not been genetically engineered and specifically contemplates various polypeptides arising from post-translational modifications of the polypeptide including, but not limited to, proteolytic processing, acetylation, carboxylation, glycosylation, phosphorylation, lipidation and acylation.

As used herein, “recombinant” refers to a polypeptide or protein, means that is derived from recombinant (e.g., microbial or mammalian) expression systems. “Microbial” refers to recombinant polypeptides or proteins made in bacterial or fungal (e.g., yeast) expression systems. As a product, “recombinant microbial” refers to a polypeptide or protein essentially unaccompanied by associated native glycosylation. Polypeptides or proteins expressed in most bacterial cultures, e.g., E. coli, will be free of glycosylation modifications; polypeptides or proteins expressed in yeast will be glycosylated.

As used herein, “isolated” refers to a nucleic acid or polypeptide separated from at least one macromolecular component (e.g., nucleic acid or polypeptide) present with the nucleic acid or polypeptide in its natural source. In one embodiment, the polynucleotide or polypeptide is purified such that it constitutes at least 95% by weight, more preferably at least 99.8% by weight, of the indicated biological macromolecules present (but water, buffers, and other small molecules, especially molecules having a molecular weight of less than 1000 daltons, can be present).

As used herein, “substantially” varies with the context as understood by those skilled in the relevant art and generally means at least 70%, preferably means at least 80%, more preferably at least 90%, and still more preferably 95%, and most preferably at least 98%.

As used herein, a “sub-inhibitory” concentration of e.g. tetracycline or a tetracycline analog refers to a concentration that does not significantly affect the growth rate of a specific prokaryotic organism. That is, the growth rate of the prokaryotic organism cultured in the presence of a sub-inhibitory concentration of tetracycline or a tetracyline analog is substantially the same as that of the same organism cultured in the absence of tetracycline or the tetracyline analog. A sub-inhibitory level of tetracycline or a tetracycline analog is also referred to herein as a “non-antibiotic” concentration of tetracycline or a tetracycline analog.

As used herein, “substantial sequence homology” as used in reference to the nucleotide sequence of DNA, the ribonucleotide sequence of RNA, or the amino acid sequence of protein, that have slight and non-consequential sequence variations from the actual sequences disclosed herein. Species having substantial sequence homology are considered to be equivalent to the disclosed sequences and as such are within the scope of the appended claims. In this regard, “slight and non-consequential sequence variations” mean that “homologous” sequences, i.e., sequences that have substantial similarity with the DNA, RNA, or proteins disclosed and claimed herein, are functionally equivalent to the sequences disclosed and claimed herein. Functionally equivalent sequences will function in substantially the same manner to produce substantially the same compositions as the nucleic acid and amino acid compositions disclosed and claimed herein. In particular, functionally equivalent DNAs encode proteins that are the same as those disclosed herein or that have conservative amino acid variations, such as substitution of a non-polar residue for another non-polar residue or a charged residue for a similarly charged residue. These changes include those recognized by those of skill in the art as those that do not substantially alter the tertiary structure of the protein.

As used herein, “substantially pure” means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, “biological activity” refers to the in vivo activities of a compound or physiological responses that result upon administration of a compound, composition or other mixture. Biological activities may be observed in in vitro systems designed to test or use such activities.

As used herein, “functionally equivalent,” refers to a polypeptide capable of exhibiting a substantially similar in vivo activity as the modified revTetR repressors encoded by one or more of the nucleotide sequences described herein.

As used herein: stringency of hybridization in determining sequence similarity is as follows:

1) high stringency: 0.1× SSPE, 0.1% SDS, 65° C.

2) moderate stringency: 0.2× SSPE, 0.1% SDS, 50° C.

3) low stringency: 1.0× SSPE, 0.1% SDS, 50° C.

It is understood that equivalent stringencies may be achieved using alternative buffers, salts and temperatures (e.g., see Maniatis (1989) Molecular Cloning, A Laboratory Manual, Cold Spring Harbor, N.Y.; Current Protocols in Molecular Biology (Ausubel et al., eds) Vol. 1, Chapter 2 (John Wiley & Sons, Inc.)).

As used herein, “expression” refers to the process by which a nucleic acid is transcribed into mRNA and translated into peptides, polypeptides, or proteins.

As used herein, “vector” or “plasmid” refers to discrete elements that are used to introduce heterologous DNA into cells for either expression of the heterologous DNA or for replication of the cloned heterologous DNA. Selection and use of such vectors and plasmids are well within the level of skill of the art.

As used herein, “transformation/transfection” refers to the process by which DNA or RNA is introduced into cells. Transfection refers to the taking up of exogenous nucleic acid, e.g., an expression vector, by a host cell whether any coding sequences are in fact expressed or not. Numerous methods of transfection are known to the ordinarily skilled artisan, for example polyethylene glycol [PEG]-mediated DNA uptake, electroporation, lipofection [see, e.g., Strauss (1996) Meth. Mol. Biol. 54:307-327], microcell fusion [see, Lambert (1991) Proc. Natl. Acad. Sci. U.S.A. 88:5907-5911; U.S. Pat. No. 5,396,767, Sawford et al. (1987) Somatic Cell Mol. Genet. 13:279-284; Dhar et al. (1984) Somatic Cell Mol. Genet. 10:547-559; and McNeill-Killary et al. (1995) Meth. Enzymol. 254:133-152], lipid-mediated carrier systems [see, e.g., Teifel et al. (1995) Biotechniques 19:79-80; Albrecht et al. (1996) Ann. Hematol. 72:73-79; Holmen et al. (1995) In Vitro Cell Dev. Biol. Anim. 31:347-351; Remy et al. (1994) Bioconjug. Chem. 5:647-654; Le Bolch et al. (1995) Tetrahedron Lett. 36:6681-6684; Loeffler et al. (1993) Meth. Enzymol. 217:599-618] or other suitable method. Transformation means introducing DNA into an organism so that the DNA is replicable, either as an extrachromosomal element or by chromosomal integration. Transformation include various processes of DNA transfer that occur between organisms, such as but not limited to conjugation. Successful transformation/transfection is generally recognized by detection of the presence of the heterologous nucleic acid within the transformed/transfected cell, such as any indication of the operation of a vector within the host cell.

As used herein, “recombinant host cells” refers to cultured cells which have stably integrated a recombinant transcriptional unit into chromosomal DNA or carry stably the recombinant transcriptional unit extrachromosomally. Recombinant host cells as defined herein will express heterologous polypeptides or proteins, particularly revTeR repressors of the present invention, and RNA encoded by the DNA segment or synthetic gene in the recombinant transcriptional unit. This term also means host cells which have stably integrated a recombinant genetic element or elements having a regulatory role in gene expression, for example, promoters or enhancers. Recombinant expression systems as defined herein will express RNA, polypeptides or proteins endogenous to the cell upon induction of the regulatory elements linked to the endogenous DNA segment or gene to be expressed. The cells can be prokaryotic or eukaryotic.

As used herein, “prokaryotic organism” includes members of Eubacteria and Archaea.

As used herein, the one letter and three letter abbreviations for amino acids are in accord with their common usage and the IUPAC-IUB Commission on Biochemical Nomenclature, see, (1972) Biochem. 11: 1726. Each naturally occurring L-amino acid is identified by the standard three letter code or the standard three letter code with or without the prefix “L-”; the prefix “D-” indicates that the stereoisomeric form of the amino acid is D.

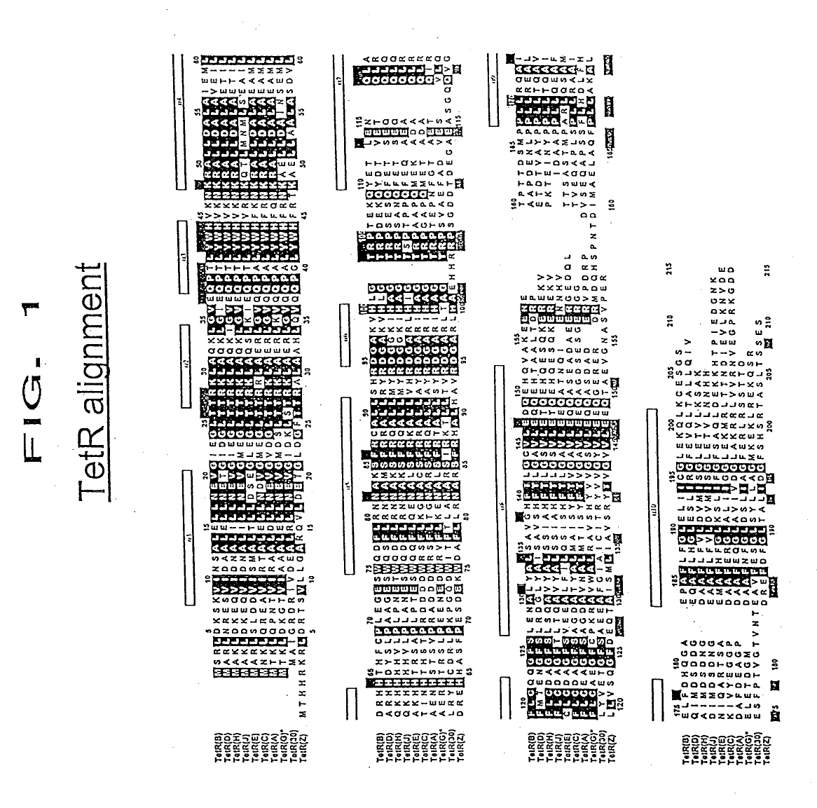

As used herein, mutations within the class B-class D chimeric modified repressor are indicated by the wild type amino acid residue, the amino acid position corresponding to SEQ ID NO: 32, and the mutant amino acid residue. For example, G96R shall mean a mutation from glycine to arginine at position 96 in the chimeric modified repressor. Mutations in other classes of repressor will be indicated by the gene, its classification, the wild type amino acid residue, the amino acid position corresponding to the representative of the class as indicated above, and as shown in FIG. 1, and the mutant amino acid residue.

5.2 Modified Tetracycline Repressors

5.2.1 Tetracycline Repressors Exhibiting a Reverse Phenotype in Prokaryotes

As used herein, “tetracycline analog” or “Tc analog” is intended to include compounds which are structurally related to tetracycline and which bind to the Tet repressor with a Kₐ of at least about 10⁻⁶ M. Preferably, the tetracycline analog binds with an affinity of about 10⁻⁹ M or greater. Examples of such tetracycline analogs include, but are not limited to, anhydrotetracycline (atc), doxycycline, chlorotetracycline, oxytetracycline and others disclosed by Hlavka and Boothe, “The Tetracyclines,” in Handbook of Experimental Pharmacology 78, R. K. Blackwood et al. (eds.), Springer-Verlag, Berlin, N.Y., 1985; L. A. Mitscher, “The Chemistry of the Tetracycline Antibiotics”, Medicinal Research 9, Dekker, N.Y., 1978; Noyee Development Corporation, “Tetracycline Manufacturing Processes” Chemical Process Reviews, Park Ridge, N.J., 2 volumes, 1969; R. C. Evans, “The Technology of the Tetracyclines,” Biochemical Reference Series 1, Quadrangle Press, New York, 1968; and H. F. Dowling, “Tetracycline,” Antibiotic Monographs, no. 3, Medical Encyclopedia, New York, 1955. For use in prokaryotic organisms, a Tc analog can be chosen which has reduced antibiotic activity as compared to Tc, such as, but not limited to, anhydrotetracycline.

As used herein, “wild-type Tet repressor” is intended to describe a protein occurring in nature which represses transcription via binding to a tet operator sequence in a prokaryotic cell in the absence of Tc. The difference(s) between a modified Tet repressor and a wild-type Tet repressor may be substitution of one or more amino acids, deletion of one or more amino acids or addition of one or more amino acids. The term is intended to include repressors of different class types, such as but not limited to, TetR(A), TetR(B), TetR(C), TetR(D), TetR(E), TetR(G), TetR(H), TetR(J), and TetR(Z).

In light of the high degree of sequence conservation (at least 80%) among members of each class of Tet repressor, a single member of each class of Tet repressor is used herein as representative of the entire class. Accordingly, the teaching of the present invention with respect to a specific member of a Tet repressor class is directly applicable to all members of that class.

As used herein, the TetR(A) class is represented by the Tet repressor carried on the Tn1721 transposon (Allmeir et al. (1992) Gene 111(1): 11-20; NCBI (National Library of Medicine, National Center for Biotechnology Information) accession number X61367 and database cross reference number (GI:) for encoded protein sequence GI:48198). This representative TetR(A) protein sequence is provided as SEQ ID NO: 34, encoded by the nucleotide sequence of SEQ ID NO: 33.

The TetR(B) class is represented by a Tet repressor encoded by a Tn10 tetracycline resistance determinant (Postle et al. (1984) Nucleic Acids Research 12(12): 4849-63, Accession No. X00694, GI:43052). This representative TetR(B) protein sequence is provided as SEQ ID NO: 36, which is encoded by the nucleotide sequence of SEQ ID NO: 35.

The TetR(C) class is represented by the tetracycline repressor of the plasmid pSC101 (Brow et al. (1985) Mol. Biol. Evol. 2(1): 1-12, Accession No. M36272, GI:150496). This representative TetR(C) protein sequence is provided as SEQ ID NO: 38, which is encoded by the nucleotide sequence of SEQ ID NO: 37.

The TetR(D) class is represented by the Tet repressor identified in Salmonella ordonez (Allard et al. (1993) Mol. Gen. Genet. 237(1-2): 301-5, Accession No. X65876, GI:49075). This representative TetR(D) protein sequence is provided as SEQ ID NO: 40, which is encoded by the nucleotide sequence of SEQ ID NO: 39.

The TetR(E) class is represented by a Tet repressor isolated from a member of Enterobacteriaceae (Tovar et al. (1988) Mol. Gen. Genet. 215(1): 76-80, Accession No. M34933, GI:155020). This representative TetR(E) protein sequence is provided as SEQ ID NO: 42, which is encoded by the nucleotide sequence of SEQ ID NO: 41.

The TetR(G) class is represented by a Tet repressor identified in Vibrio anguillarum (Zhao et al. (1992) Microbiol Immunol 36(10): 1051-60, Accession No. S52438, GI:262929). This representative TetR(G) protein sequence is provided as SEQ ID NO: 44, which is encoded by the nucleotide sequence of SEQ ID NO: 43.

The TetR(H) class is represented by a Tet repressor encoded by plasmid pMV111 isolated from Pasteurella multocida (Hansen et al. (1993) Antimicrob. Agents. Chemother. 37(12): 2699-705, Accession No. U00792, GI:392872). This representative TetR(H) protein sequence is provided as SEQ ID NO: 46, which encoded by the nucleotide sequence of SEQ ID NO: 45.

The TetR(J) class is represented by a Tet repressor cloned from Proteus mirabilis (Magalhaes et al. (1998) Biochim. Biophys. Acta. 1443(1-2): 262-66, Accession No. AF038993, GI:4104706). This representative TetR(J) protein sequence is provided as SEQ ID NO: 48, which is encoded by the nucleotide sequence of SEQ ID NO: 47.

The TetR(Z) class is represented by a Tet repressor encoded by the pAG1plasmid isolated from the gram-positive organism Corynebacterium glutamicum (Tauch et al. (2000) Plasmid 44(3): 285-91, Accession No. AAD25064, GI:4583400). This representative TetR(Z) protein sequence is provided as SEQ ID NO: 50, which is encoded by the nucleotide sequence of SEQ ID NO: 49.

As used herein, “tet operator,” “tet operator sequence,” or tetO, is intended to encompass all classes of tet operator sequences, such as but not limited to tetO(A), tetO(B), tetO(C), tetO(D), tetO(E), tetO(G), tetO(H), tetO(J) and tetO(Z). The nucleotide sequences of Tet repressors of members of the A, B, C, D, E, G, H, J and Z classes, and their corresponding tet operator sequences are known, and can be used in the present invention. See, for example, Waters, S. H. et al. (1983) Nucl. Acids Res 11:6089-6105, Hillen, W. and Schollmeier, K. (1983) Nucl. Acids Res. 11:525-539 and Postle, K. et al. (1984) Nucl. Acids Res. 12:4849-4863, Unger, B. et al. (1984) Gene 31: 103-108, Unger, B. et al. (1984) Nucl Acids Res. 12:7693-7703 and Tovar, K. et al. (1988) Mol. Gen. Genet. 215:76-80, which are incorporated herein by reference in their entireties.

As used herein, “modified tetracycline repressor,” “modified tetracycline repressor exhibiting a reverse phenotype,” “revTetR,” or “revTetR protein” is intended to include polypeptides having an amino acid sequence which is similar to one or more wild-type Tet repressor but which has at least one amino acid difference from a wild-type Tet repressor that confers greater binding affinity to a tet operator sequence in prokaryotes in the presence of tetracycline or its analog than in the absence of tetracycline or its analog. A revTetR provided herein has the following functional properties: 1) the polypeptide can bind to a tet operator sequence, i.e., it retains the DNA binding specificity of a wild-type Tet repressor; and 2) it is regulated in a reverse manner by tetracycline than a wild-type Tet repressor, i.e., the modified Tet repressor binds to a tet operator sequence with a greater binding affinity (or a lower dissociation constant, K_{d}) in the presence of Tc or Tc analog, than in the absence of Tc or its analog. Moreover, the affinity of a revTetR protein of the present invention for a tet operator sequence is substantially proportional to the concentration of tetracyline; that is, as the concentration of tetracycline or analog thereof increases, the binding affinity of the revTetR protein for the tet operator sequence increases. Preferably, this reverse phenotype of the revTetR is only displayed in a prokaryote, and not in a eukaryote. The term modified tetracycline repressor or revTetR is intended to include modified TetR of different class types, such as but not limited to TetR(A), TetR(B), TetR(C), TetR(D), TetR(E), TetR(G), TetR(H), TetR(J), and TetR(Z), as well as “chimeric tetracycline repressor” or “chimeric revTetR”.

As used herein, “chimeric tetracycline repressor” or “chimeric revTetR” is intended to include polypeptides having an amino acid sequence comprising amino acid residues derived from more than one type of tetracycline repressor and exhibits the reverse phenotype in prokaryotes. The term is intended to include chimeric tetracycline repressors constructed from different class types, such as but not limited to, TetR(A), TetR(B), TetR(C), TetR(D), TetR(E), TetR(G), TetR(H), TetR(J), and TetR(Z). In certain embodiments, the chimeric tetracycline repressors of the present invention comprise an amino-terminal DNA-binding domain and a carboxy-terminal tetracycline binding domain, including but not limited to the corresponding domains of the TetR(A), TetR(B), TetR(C), TetR(D), TetR(E), TetR(G), TetR(H), TetR(J), and TetR(Z). Such chimeric tetracycline repressors further comprise at least one amino acid substitution that confers the reverse phenotype. A chimeric revTetR retains the DNA binding specificity of the DNA binding domain of a wild-type Tet repressor. Preferably, this reverse phenotype of the chimeric revTetR is only displayed in a prokaryote, and not in a eukaryote. In preferred embodiments, the chimeric revTetR is a “TetR(BD)” comprising about amino acids 1 to 50 from TetR(B) (SEQ ID NO: 36) operatively linked to amino acid residues about 51 to 208 of TetR(D) (SEQ ID NO: 40) and that further comprises at least one substitution that confers binding to DNA containing a tet operator sequence with a greater affinity (i.e., lower dissociation constant K_{d}) in the presence of a tetracycline (Tc) or tetracycline analog, compared to DNA binding in the absence of a tetracycline (Tc) or tetracycline analog.

The term “modified tetracycline repressor” or “modified revTetR” further include Tet repressors wherein the amino-terminal DNA-binding domain is derived from a DNA-binding protein other than a TetR repressor protein, and the DNA sequence to which such a chimeric tetracycline repressor protein binds corresponds to the DNA sequence recognized and bound by the non-TetR repressor, DNA-binding protein. Non-limiting examples of such DNA-binding proteins include, but are not limited to, the cro repressor, 454 repressor and CI repressor of bacteriophage λ, as well as the hin, gin, cin, and pin recombinase proteins (see, Feng et al. (1994) Science 263: 348-55).

In a preferred embodiment, the parent Tet repressors from which the chimeric repressors of the present invention are constructed are TetR of classes B and D (see Schnappinger et al., (1998) EMBO J. 17:535-543), and the tet operator sequence is a class B tet operator sequence.

In preferred embodiments, the “modified tetracycline repressor” or “modified revTetR” or “chimeric revTetR” of the present invention is not a fusion protein comprising a protein or protein portion that activates transcription in a eukaryotic cell.

As described in detail below, the inventor discovered that revTetR that are active in prokaryotic organisms have amino acid substitutions that tend to be localized in discrete regions of the polypeptide sequence. In particular, the inventor discovered that nucleotide substitutions that result in at least one codon change in amino acid residues from positions 70, 71, 91 to 103, 157-159 and 192 to 205 of SEQ ID NO: 32 appear to be important for the reverse phenotype in prokaryotic organisms. In addition, nucleotide substitutions that result in at least one codon change in amino acid residues found within the following regions also appear to be important for the reverse phenotype in prokaryotic organisms: residues from positions 13-25, more specifically 14-24, and even more specifically residues from positions 17-23, 53-61, and/or 95-99 of SEQ ID NO: 32.

The crystal structure of a Tet repressor-tetracycline complex, as described in Hinrichs, W. et al. (1994) Science 264: 418-420, can be used for the rational design of mutant Tet repressors. The polypeptide folds into 10 alpha helices, α1 to α10. Helices α7 to α10 are apparently involved in the dimerization of the repressor. More specifically, Hinrichs further described the tetracycline repressor protein as made up of a “protein core” and DNA binding domains. The DNA core comprises α-helices α5 to α10. The tetracycline binding pocket is formed with the carboxy-termini of the α4 and α6 helices along with the α5, α7, α8′, and α9′ helices (where the prime indicates that the helix is part of the second repressor of the DNA-binding and tetracycline-binding dimer). The DNA binding domains are formed with α helices α1-α3 of both repressor proteins of the dimer and the DNA-binding domains are connected to the core through the α4 helix. The amino sequence of each of the ten α helices of the TetR(B) and TetR(D) are provided in Schnappinger et al. (1998) EMBO J. 17(2): 535-543. Accordingly, each of these ten helices appears to include the following indicated amino acid residues as provided in SEQ ID NO: 32: α1, amino acid residues 5-21; α2, amino acid residues 27-34; α3, amino acid residues 38-44; α4, amino acid acid residues 48-64; α5, amino acid residues 74-92; α6, amino acid residues 95-100; α7, amino acid residues 110-123; α8, amino acid residues 128-154; α9, amino acid residues 167-178; and α10, amino acid residues 183-203.

Therefore, based upon the crystal structure, amino acid positions 70 and 71 are located prior to α5 of the tetracycline-binding pocket and yet amino acid substitutions at this site appear to contribute to the desired functional properties of a revTetR. Moreover, amino acid positions 95, 96, 98, 101 and 103 located within a6 that forms a part of the conserved tetracycline-binding pocket, and amino acid positions 188, 192, 196 and 200 located within α10 also appear to be involved in conferring the reverse phenotype to a revTetR. In addition, as demonstrated below amino acid substitutions within the peptide sequence within or adjacent to the α1 helix involved in DNA binding, i.e. spanning amino acids 13-25, particularly 14-24, and more particularly 17-23, especially residues 18, 20, and 22, and even more particularly, residue 18, appear to contribute to the desired functional properties of a revTetR. Similarly, amino acid substitutions within the α4 helix involved in tetracycline binding as well as connecting the DNA-binding domain to the core protein, i.e. the peptide sequence spanning amino acids 53-61, particularly residues 53, 56, 59, and 61, and more particularly amino acid residues 56 and 59, appear to contribute to the desired functional properties of a revTetR. Moreover, amino acid substitutions within the α6 helix which, as noted above, forms part of the conserved tetracycline-binding pocket, i.e. the peptide sequence spanning amino acids 95-99, particularly amino acid residues 99 and 96, appear to contribute to the desired functional properties of a revTetR. These observations suggest, without being bound by any theory, that these mutations may alter the relative position of the monomers in the dimer or alter the resulting conformation or relative position of the DNA binding domain such that upon binding of tetracycline or tetracycline analog, the proper conformation for binding to DNA is restored, rather than perturbed.

Accordingly, in certain embodiments, the modified tetracycline repressor polypeptides exhibiting a reverse phenotype in prokaryotic organisms of the present invention comprise at least one, at least two, or at least three amino acid substitutions within any helix of helices α1-α10 of a tetracycline repressor protein.

5.2.2 Exemplary Modified Repressors

In one embodiment, the modified tetracycline repressor polypeptide is the TetR(BD) chimera (SEQ ID NO. 32) further comprising at least one amino acid substitution at position 96 or 99, or substitutions at positions 96, 103 and 114; positions 96, 157 and 200; positions 96 and 159; positions 160, 178, 196; positions 59, 95 and 100; positions 96 and 188; positions 96 and 205; positions 96 and 110; positions 99 and 194; positions 99 and 158; positions 70, 91 and 99; positions 71, 95 and 127; positions 59, 98, 101 and 192.

Presently preferred amino acid substitutions that confer a reverse phenotype in prokaryotes in a TetR(BD) chimera include, but are not limited to, Asn at position 59, Val at positions 70 and 71; Gln at position 91; Glu and Gly at position 95; Arg and Glu at position 96; Arg at position 98; Glu at position 99; Ala at position 100; His at position 101; Ser at position 103; Phe at position 110; Val at position 114; Arg at position 127; Asn at position 157; Cys at position 158; Leu at position 159; Gln at position 188; Gly at position 192; Val at position 194; Trp at position 196; His at position 200; and Ser at position 205. In more preferred embodiments, the revTetR repressor polypeptide is selected from any of the amino acid sequences set forth in SEQ ID NOS. 2, 4, 6, 8, 10, 12, 14, 16, 18, 20, 22, 24, 26, 28 and 30.

Additional amino acid substitutions that confer a reverse phenotype in prokaryotes in a TetR(BD) chimera include those amino acid substitutions provided in Table 3. Accordingly, revTetR polypeptides of the present invention are also selected from those comprising an amino acid sequence selected from the group consisting of SEQ ID NOS.: 71 to 264.

Table 1 discloses the designation (TetRev) of specific RevTetR-containing isolates and the corresponding SEQ ID NO. and amino acid substitution(s) present in those isolates, as compared with the amino acid sequence of the corresponding wild-type chimeric tetracycline repressor protein (SEQ ID NO: 32).

TABLE 1

| Mutant Name | SEQ ID NO: | Amino Acid Substitutions | | | | | | |
|---|---|---|---|---|---|---|---|---|
| TetRrevAtc4-1 | 71 | A50S | D53F | A56G | V57I | H63Q | | |
| TetRrevAtc4-10 | 72 | I59N | L60F | A61G | | | | |
| TetRrevAtc4-11 | 73 | L55L | A56P | R62S | | | | |
| TetRrevAtc4-13 | 74 | E58N | I59T | L60F | H63Y | | | |
| TetRrevAtc4-14 | 75 | L51L | D53Q | V57V | E58K | L60L | A61V | R62P |
| | | H63D | | | | | | |
| TetRrevAtc4-16 | 76 | D53N | A54G | V57F | I59S | L60F | | |
| TetRrevAtc4-17 | 77 | L52M | A56P | V57L | E58V | | | |
| TetRrevAtc4-18 | 78 | E58N | I59T | L60F | H63Y | | | |
| TetRrevAtc4-19 | 79 | R49H | A56P | V57L | L60M | | | |
| TetRrevAtc4-2 | 80 | R49G | L55L | V57V | E58N | R62L | H63Q | |
| TetRrevAtc4-20 | 81 | V57V | E58K | L60V | A61T | R62G | | |
| TetRrevAtc4-21 | 82 | K6K | D53F | L55L | V57L | I59S | A61S | |
| TetRrevAtc4-22 | 83 | R49H | A56P | V57L | L60M | | | |
| TetRrevAtc4-23 | 84 | A50D | L51V | L52Q | A54A | L55L | A61S | |
| TetRrevAtc4-24 | 85 | A56P | V57A | E58Q | H63Q | | | |
| TetRrevAtc4-25 | 86 | K48K | V57L | E58V | I59T | L60F | R62S | |
| TetRrevAtc4-28 | 87 | V57V | E58K | L60V | R62G | | | |
| TetRrevAtc4-29 | 88 | E58N | I59T | L60F | H63Y | | | |
| TetRrevAtc4-3 | 89 | D53E | A54S | I59M | A61P | | | |
| TetRrevAtc4-31 | 90 | A50S | D53Y | A56G | V57I | H63Q | | |
| TetRrevAtc4-37 | 91 | R49R | L52L | D53E | A54S | L55L | A61P | |
| TetRrevAtc4-4 | 92 | I59S | R62S | | | | | |
| TetRrevAtc4-40 | 93 | A56P | L60L | A61G | R62R | | | |
| TetRrevAtc4-43 | 94 | L52V | A56V | V57L | I59T | A61S | | |
| TetRrevAtc4-44 | 95 | D53Y | L55M | A56C | L60F | A61A | H63N | |
| TetRrevAtc4-47 | 96 | L51L | E58A | I59N | H63Y | | | |
| TetRrevAtc4-48 | 97 | D53N | A54S | L55L | A56P | V57V | A61A | R62L |
| TetRrevAtc4-5 | 98 | I59F | A61S | H64Q | A97T | | | |
| TetRrevAtc4-52 | 99 | D53F | A54T | L55M | I59S | H63Q | | |
| TetRrevAtc4-53 | 100 | D53F | L55M | I59S | H63Q | | | |
| TetRrevAtc4-59 | 101 | D53Y | L55M | A56C | L60F | H63N | | |
| TetRrevAtc4-6 | 102 | D53Y | L55L | A56P | R62L | | | |
| TetRrevAtc4-61 | 103 | L51L | A56V | I59H | R62L | | | |
| TetRrevAtc4-67 | 104 | K48K | D53Y | A56G | E58G | | | |
| TetRrevAtc4-7 | 105 | I59R | L55L | A61E | H64N | | | |
| TetRrevAtc4-70 | 106 | D53A | A61P | | | | | |
| TetRrevAtc4-71 | 107 | D53T | L55M | 56P | A61S | | | |
| TetRrevAtc4-8 | 108 | E58N | I59T | L60F | H63Y | | | |
| TetRrevAtc4-9 | 109 | L52M | A56P | V57L | E58V | L60L | H188Y | |
| TetRrevAtc4-9b | 110 | A56P | V57L | E58V | L60L | | | |
| TetRrevDox4-1 | 111 | D53E | A54S | A61P | | | | |
| TetRrevDox4-2 | 112 | D53E | A54S | I59M | A61P | | | |
| TetRrev04-1 | 113 | G96E | K98T | | | | | |
| TetRrev04-4 | 114 | G96E | V113A | | | | | |
| TetRrev6-13 | 115 | G96R | L101I | | | | | |
| TetRrev6-17 | 116 | R94G | G96W | | | | | |
| TetRrev6-2 | 117 | D95G | G96R | H100L | | | | |
| TetRrev6-30 | 118 | G96Q | G102S | | | | | |
| TetRrev6-23 | 119 | D95E | A97E | | | | | |
| TetRrev6-25 | 120 | Y93S | D95E | G102A | | | | |
| TetRrev6-26 | 121 | Y93F | G96W | A97E | H100N | | | |
| TetRrev6-27 | 122 | A71T | G96E | | | | | |
| TetRrev6-28 | 123 | G96R | A97S | V99L | | | | |
| TetRrev6-29 | 124 | A97T | K98R | V99G | | | | |
| TetRrev6-3 | 125 | V99G | H100P | | | | | |
| TetRrev6-31 | 126 | G96Q | G102S | | | | | |
| TetRrev6-32 | 127 | L79V | A97T | K98R | V99G | | | |
| TetRrev6-33 | 128 | A97T | K98R | V99G | | | | |
| TetRrev6-34 | 129 | R94L | D95A | H100 | | | | |
| TetRrev6-35 | 130 | D95Y | V99E | | | | | |
| TetRrev6-36 | 131 | R94L | D95A | H100Q | | | | |
| TetRrev6-37-1 | 132 | G96R | A97S | V99L | | | | |
| TetRrev6-38 | 133 | Y93H | G96W | K98Q | V99G | | | |
| TetRrev6-39 | 134 | G96Q | G102S | | | | | |
| TetRrev6-40 | 135 | Y93N | G96W | V99G | | | | |
| TetRrev6-50 | 136 | V99G | | | | | | |
| TetRrev6-51 | 137 | A97T | V99G | | | | | |
| TetRrev6-53 | 138 | A97P | K98R | V99D | | | | |
| TetRrev6-54 | 139 | Y93F | D95N | V99F | | | | |
| TetRrev4/6-3 | 140 | D95A | V99G | | | | | |
| TetRrev4/6-4 | 141 | D95A | V99G | V57V | A61G | | | |
| TetRrev4/6-5 | 142 | A54S | V99R | | | | | |
| TetRrev4/6-6 | 143 | D53Y | V57V | A61P | | | | |
| TetRrev4/6-7 | 144 | L55L | L60V | A97V | V99G | | | |
| TetRrev4/6-10 | 145 | A56S | V99E | | | | | |
| TetRrev4/6-15 | 146 | A56S | I59A | L60W | V99V | | | |
| TetRrev4/6-17 | 147 | G99R | V99L | L101I | | | | |
| TetRrev4/6-24 | 148 | A61T | V99R | | | | | |
| TetRrev4/6-25 | 149 | I59S | L60L | R62G | D95A | V99L | | |
| TetRrev4/6-27 | 150 | V99R | | | | | | |
| TetRrev1/34: | 151 | L17S | E23K | | | | | |
| TetRrev3/38 | 152 | L17H | I22F | E23G | | | | |
| TetRrev19/48 | 153 | N18K | E19D | V20L | I22N | E23A | | |
| TetRrev22/5 | 154 | E15V | L17A | E19D | | | | |
| TetRrev25/43 | 155 | L17F | E19- | G24G | | | | |
| TetRrev28/8 | 156 | L14S | N18Y | I22F | L25L | | | |
| TetRrev28/16 | 157 | L14L | E15A | L17G | L25V | | | |
| TetRrev28/23 | 158 | A13A | L14S | N18K | E23V | | | |
| TetRrev28/26 | 159 | L14T | N18Y | G21G | I22F | | | |
| TetRrev28/27 | 160 | L17G | I22M | | | | | |
| TetRrev28/30 | 161 | V20G | G21A | I22I | E23G | | | |
| TetRrev28/31 | 162 | A13A | V20G | G21R | I22N | G24G | | |
| TetRrev28/36 | 163 | N18I | E19G | V20G | E23V | | | |
| TetRrev28/40 | 164 | L14S | N18Y | I22F | L25L | | | |
| TetRrev28/41 | 165 | L17L | N18D | V20R | I22N | | | |
| TetRrev28/46 | 166 | A13A | V20G | G21R | I22N | G24G | | |
| TetRrev28/48 | 167 | N18Y | V20D | I22T | W43S | | | |
| TetRrev28/49 | 168 | N18Y | V20D | I22T | | | | |
| TetRrev29/9 | 169 | L17V | N18Y | G21G | I22T | | | |
| TetRrev29/17 | 170 | L17S | E23D | | | | | |
| TetRrev29/24 | 171 | E15V | L17F | N18Y | I22M | E23K | | |
| TetRrev29/25 | 172 | G21- | I22- | E23- | G24S | | | |
| TetRrev29/27 | 173 | L14E | V20G | | | | | |
| TetRrev29/35 | 174 | L17S | E23D | | | | | |
| TetRrev29/42 | 175 | E19D | G96R | | | | | |
| TetRrev29/44 | 176 | A13A | V20G | G21R | I22N | G24G | | |
| TetRrev29/52 | 177 | L14V | L17V | N18K | V20V | G24G | | |
| TetRrevAD1/2 | 178 | N18Y | L52L | D53Y | A54A | I59T | | |
| TetRrevAD1/6 | 179 | N18Y | E23V | D53A | A54S | L55L | A56S | A61T |
| | | H63Y | | | | | | |
| TetRrevAF1/7 | 180 | G96R | L101I | | | | | |
| TetRrevAF1/8 | 181 | G24G | Y93N | L101F | G102D | | | |
| TetRrevAF1/11 | 182 | L14I | V20V | I22L | R94H | A97A | V99E | |
| TetRrevAF2/5 | 183 | L14R | E!9E | D95H | L101P | | | |
| TetRrevAD2/4 | 184 | L17F | N18R | D53N | I59M | A61T | | |
| TetRrevAD2/6 | 185 | N18Y | G21E | E23K | | | | |
| TetRrevAD2/12 | 186 | E23H | D53Y | A56A | E58Q | I59T | | |
| TetRrevAD2/13 | 187 | E19D | V20E | E23K | V57L | I59N | L60F | A61A |
| TetRrevAD2/2 | 188 | L14I | G21R | A56V | | | | |
| TetRrevAF1/3 | 189 | G24G | D95A | G96R | | | | |
| TetRrevAF1/4 | 190 | N18K | D95V | G96V | A97V | | | |
| TetRrevAF1/5 | 191 | L14V | G24G | Y93D | R94G | D95G | | |
| TetRrevAD3/2 | 192 | A56P | E58H | A61A | H63Y | | | |
| TetRrevAD3/3 | 193 | G21- | I22- | E23- | G24S | | | |
| TetRrevAF2/7 | 194 | Y93C | D95E | A97T | | | | |
| TetRrevAF6/12 | 195 | L14F | E15E | G96V | K98E | V99L | L101P | |
| TetRrevAF7/1 | 196 | Y93C | G96R | K98N | | | | |
| TetRrevAF7/2 | 197 | L14V | E15V | E23D | K48K | G96L | G102A | |
| TetRrevAD3/5 | 198 | L17L | N18K | E19V | I22T | 53Y | A54S | |
| TetRrevAD3/6 | 199 | V20G | A56P | I59L | R62R | | | |
| TetRrevAD3/7 | 200 | L17M | E19D | L52L | D53Y | A54A | A56E | |
| TetRrevAD3/8 | 201 | L16L | A56A | V57E | E58L | I59N | L60A | |
| TetRrevAF2/14 | 202 | L14L | E15A | L16L | L17V | N18N; | V20V | G96V |
| TetRrevAF2/15 | 203 | V20G | I22N | R49Q | A50T | | | |
| TetRrevAF2/16 | 204 | E19V | Y93C | D95Y | A97T | K98N | V99L | L101L |
| TetRrevAF3/5 | 205 | V20F | G21R | R49S | | | | |
| TetRrevAD3/9 | 206 | E15Q | L17L | L55M | I59R | | | |
| TetRrevAD3/10 | 207 | N18Y | G24G | L51L | D53Y | A54V | | |
| TetRrevAD3/11 | 208 | L14V | G24G | Y93D | R94G | D95G | | |
| TetRrevAF3/6 | 209 | E23K | D95H | L101H | | | | |
| TetRrevAF3/7 | 210 | L17H | I22F | A97P | | | | |
| TetRrevAF3/8 | 211 | E15V | L16L | N18H | E19D | V20D | R94P | D95H |
| | | H100N | | | | | | |
| TetRrevAF3/10 | 212 | G96G | V99G | H100P | | | | |
| TetRrevAF4/1 | 213 | E15V | V20A | I22F | E23Q | L25L | G96V | K98E |
| | | G102G | | | | | | |
| TetRrevAF4/3 | 214 | L16Q | N18H | E19V | V20G | I22I | G96T | |
| TetRrevAF4/4 | 215 | G24A | R94P | D95E | G102D | | | |
| TetRrevAF4/5 | 216 | E15G | L16L | D95A | V99G | | | |
| TetRrevAF4/6 | 217 | E15D | L17I | N18T | E23G | A54V | D95A | |
| TetRrevAF4/7 | 218 | L14V | A97T | V99G | | | | |
| TetRrevAF4/8 | 219 | L14F | E23A | G96M | A97P | L101P | | |
| TetRrevAF4/9 | 220 | L16R | E19D | E23D | D95G | V99A | G102G | |
| TetRrevAD2/5 | 221 | L17F | L55L | L60F | R62V | | | |
| TetRrevAD2/8 | 222 | V20G | D53N | A54A | A56G | V57L | I59F | L60L |
| | | R62R | | | | | | |
| TetRrevAD2/1 | 223 | G21G | L51L | D53Y | L55L | A56P | A61E | |
| TetRrevAF5/10 | 224 | R94P | G96VO | A97T | K98N | H100Q | | |
| TetRrevAF4/12 | 225 | L16Q | N18Y | A97G | H100S | | | |
| TetRrevAF4/13 | 226 | A13A | E15D | L17L | E19V | I22I | E23K | R94H |
| | | G95N | | | | | | |
| TetRrevAF5/1 | 227 | L14F | D95A | V99G | | | | |
| TetRrevAF5/3 | 228 | E15G | N18K | R94H | G96G | G102V | | |
| TetRrevAF5/5 | 229 | L14V | L17F | V20A | I22M | L25F | D95H | |
| TetRrevAF5/6 | 230 | N18H | L25F | A97P | K98N | L101H | | |
| TetRrevAF5/7 | 231 | E15A | G96M; | A97P | L101P | | | |
| TetRrevAF5/8 | 232 | I22I | R94P | V99E | | | | |
| TetRrevAF5/9 | 233 | R94C | V99E | | | | | |
| TetRrevAF5/11 | 234 | N18K | E19A | L101L | G102S | | | |
| TetRrevAF5/13 | 235 | L14L | V20G | V99E | | | | |
| TetRrevAF6/1 | 236 | A13A | E19V | V20A | D95I | G96G | | |
| TetRrevAF6/2 | 237 | N18D | 23A | D95N | H100P | L101S | | |
| TetRrevAF6/3 | 238 | G96G | V99G | H100P | | | | |
| TetRrevAF6/4 | 239 | Y93Y | G96R | A97P | | | | |
| TetRrevAF6/5 | 240 | V20V | I22V | E23D | D95N | H100P | | |
| TetRrevAF6/6 | 241 | D95Y | G96E | V99V | | | | |
| TetRrevAF6/7 | 242 | G96G | V99G | H100P | | | | |
| TetRrevAF6/8 | 243 | A71T | G96E | | | | | |
| TetRrev96/99-1 | 244 | G96H | V99R | | | | | |
| TetRrev96/99-2 | 245 | G99K | V99A | | | | | |
| TetRrev96/99-3 | 246 | G96E | V99T | | | | | |
| TetRrev96/99-4 | 247 | G96P | V99S | | | | | |
| TetRrev96/99-5 | 248 | G96I | V99K | | | | | |
| TetRrev96/99-6 | 249 | G96N | V99Q | | | | | |
| TetRrev96/99-7 | 250 | G96L | V99K | | | | | |
| TetRrev96/99-8 | 251 | G96N | V99H | | | | | |
| TetRrev96/99-9 | 252 | G96H | V99N | | | | | |
| TetRrev96/99-10 | 253 | G96N | V99P | | | | | |
| TetRrev96/99-11 | 254 | G96R | V99Y | | | | | |
| TetRrev96/99-12 | 255 | G96H | V99Q | | | | | |
| TetRrev96/99-13 | 256 | G96T | V99D | | | | | |
| TetRrev96/99-14 | 257 | G96N | V99N | | | | | |
| TetRrev96/99-15 | 258 | G96P | V99P | | | | | |
| TetRrev96/99-16 | 259 | G96P | V99Y | | | | | |
| TetRrev96/99-17 | 260 | G96T | V99K | | | | | |
| TetRrev96/99-18 | 261 | G96T | V99P | | | | | |
| TetRrev96/99-19 | 262 | G96R | V99S | | | | | |
| TetRrev96/99-20 | 263 | G96S | V99K | | | | | |
| TetRrev96P | 264 | G96P | | | | | | |

Nucleotide substitutions within the nucleic acid sequence of SEQ ID NO: 31 that confer a reverse phenotype on the encoded tetracycline repressor protein and that correspond to the mutants listed in Table 1, are provided in Table 2, which discloses the designation (TetRev) of specific RevTetR-containing isolates and the corresponding SEQ ID NO. and nucleotide substitution(s) present in those isolates, as compared with the nucleotide sequence encoding the corresponding wild-type chimeric tetracycline repressor protein (SEQ ID NO: 31).

TABLE 2

| | SEQ ID | | | | | | |
|---|---|---|---|---|---|---|---|
| Mutant Name | NO: | Preferred Nucleotide Substitutions | | | | | |
| TetRrevAtc4-1 | 265 | gcc50tcc | gat53ttt | gcg56ggg | gtg57att | cat63cag | |
| TetRrevAtc4-10 | 266 | atc59aac | ttg60ttt | gcg61ggg | | | |
| TetRrevAtc4-11 | 267 | ctg55ctt | gcg56cct | cgt62agt | | | |
| TetRrevAtc4-13 | 268 | gag58aat | atc59acc | ttg60ttc | cat63tat | | |
| TetRrevAtc4-14 | 269 | cta51ctc | gat53caa | gtg57gtt | gag58aag | tta60tta | gcg61gtg |
| | | cgt62cct | cat63gac | | | | |
| TetRrevAtc4-16 | 270 | gat63aac | gcg54ggg | gtg57ttt | atc59agc | ttg60ttt | |
| TetRrevAtc4-17 | 271 | ctg52atg | gcg56ccg | gtg57ttg | gag58gtg | | |
| TetRrevAtc4-18 | 272 | gat58aat | atc59acc | ttg60ttc | cat63tat | | |
| TetRrevAtc4-19 | 273 | cgg49cat | gcg56cct | gtg57ctg | ttg60atg | | |
| TetRrevAtc4-2 | 274 | cgg49ggc | ctg55ttg | gtg57gtt | gag58aac | cgt62ctt | cat63caa |
| TetRrevAtc4-20 | 275 | gtg57gtt | gag58aag | ttg60gtt | gcg61acg | cgt62ggt | |
| TetRrevAtc4-21 | 276 | aaa6aag | gat53ttt | ctg55ctt | gtg57ctg | atc59agc | gcg61tcg |
| TetRrevAtc4-22 | 277 | cgg49cat | gcg56cct | gtg57ctg | ttg60atg | | |
| TetRrevAtc4-23 | 278 | gcc50gac | cta51gta | ctg52cag | gcg54gct | ctg55ctt | gcg61tcg |
| TetRrevAtc4-24 | 279 | gcg56gtg | gtg57gcg | gag58cag | cat63caa | | |
| TetRrevAtc4-25 | 280 | aag48aaa | gtg57ttg | gag58gtg | atc59acc | ttg60ttt | cgt62agt |
| TetRrevAtc4-28 | 281 | gtg57gtc | gag58aag | ttg60gtt | cgt62ggt | | |
| TetRrevAtc4-29 | 282 | gag58aat | atc59acc | ttg60ttc | cat63tat | | |
| TetRrevAtc4-3 | 283 | gat53gag | gcg54tcg | atc59atg | gcg61ccc | | |
| TetRrevAtc4-31 | 284 | gcc50tcc | gat53tat | gcg56ggg | gtg57att | cat63cag | |
| TetRrevAtc4-37 | 285 | cgg49cgt | ctg52ctt | gat53gaa | gcg54tct | ctg55ctc | gcg61ccg |
| TetRrevAtc4-4 | 286 | atc59agc | cgt62agc | | | | |
| TetRrevAtc4-40 | 287 | gcg56ccc | ttg60ctc | gcg61ggg | cgt62cgc | | |
| TetRrevAtc4-43 | 288 | ctg52gtg | gcg56gtg | gtg57ttg | atc59acc | gcg61tcg | |
| TetRrevAtc4-44 | 289 | gat53tat | ctg55atg | gcg56tgc | ttg60ttc | gcg61gct | cat63aat |
| TetRrevAtc4-47 | 290 | cta51ctc | gag58gcg | atc59aac | cat63tac | | |
| TetRrevAtc4-48 | 291 | gat53aat | gcg54tcg | ctg54ctc | gcg56ccg | gtg57gta | gcg61gct |
| | | cgt62ctc | | | | | |
| TetRrevAtc4-5 | 292 | atc59ttt | gcg61tcg | cat64caa | gca97aca | | |
| TetRrevAtc4-52 | 293 | gat53ttt | gcg54acg | ctg55atg | atc59agc | cat63caa | |
| TetRrevAtc4-53 | 294 | gat53ttt | ctg55atg | atc59agc | cat63caa | | |
| TetRrevAtc4-59 | 295 | gat53tat | ctg55atg | gcg56tgc | ttg60ttc | cat63aat | |
| TetRrevAtc4-6 | 296 | gat53tat | ctg55ttg | gcg56ccg | cgt62ctt | | |
| TetRrevAtc4-61 | 297 | cta51ctt | gcg56gtg | atc59cac | cgt62ctt | | |
| TetRrevAtc4-67 | 298 | aag48aaa | gat53tat | gcg56ggg | gag58ggg | | |
| TetRrevAtc4-7 | 299 | atc59agg | ctg55ctt | gcg61gag | cat64aat | | |
| TetRrevAtc4-70 | 300 | gat53gct | gcg61ccg | | | | |
| TetRrevAtc4-71 | 301 | gat53acc | ctg55atg | gcg56ccg | gcg61tcg | | |
| TetRrevAtc4-8 | 302 | gag58aat | atc59acc | ttg60ttc | cat63tat | | |
| TetRrevAtc4-9 | 303 | ctg52atg | gcg56ccg | gtg57ttg | gag58gtg | ttg60ctg | cat188tat |
| TetRrevAtc4-9b | 304 | gcg56ccg | gtg57ttg | gag58gtg | ttg60ctg | | |
| TetRrevDox4-1 | 305 | gat53gaa | gcg54tct | gcg61ccg | | | |
| TetRrevDox4-2 | 306 | gat53gag | gcg54tcg | atc59atg | gcg61ccc | | |
| TetRrev04-1 | 307 | ggg96gag | aaa98aca | | | | |
| TetRrev04-4 | 308 | ggg96gag | gtg113gcg | | | | |
| TetRrev6-13 | 309 | ggg96agg | ctc101atc | | | | |
| TetRrev6-17 | 310 | cgt94ggt | ggg96tgg | | | | |
| TetRrev6-2 | 311 | gac95ggc | ggg96agg | cac100ctc | | | |
| TetRrev6-30 | 312 | ggg96cag | ggc102agc | | | | |
| TetRrev6-23 | 313 | gac95gaa | gca97gaa | | | | |
| TetRrev6-25 | 314 | tac93tcc | gac95gaa | ggc102gcc | | | |
| TetRrev6-26 | 315 | tac93ttc | ggg96tgg | gca97gaa | cac100aac | | |
| TetRrev6-27 | 316 | gcg71acg | ggg96gag | | | | |
| TetRrev6-28 | 317 | ggg96agg | gca97tca | gtg99ctg | | | |
| TetRrev6-29 | 318 | gca97act | aaa98aga | gtg99ggg | | | |
| TetRrev6-3 | 319 | gtg99ggg | cac100ccc | | | | |
| TetRrev6-31 | 320 | ggg96cag | ggc102agc | | | | |
| TetRrev6-32 | 321 | ctg79gtg | gca97act | aaa98aga | gtg99ggg | | |
| TetRrev6-33 | 322 | gca97act | aaa98aga | gtg99ggg | | | |
| TetRrev6-34 | 323 | cgt94ctt | gac95gcc | cac100cag | | | |
| TetRrev6-35 | 324 | gac95tat | gtg99gag | | | | |
| TetRrev6-36 | 325 | cgt94ctt | gac95gcc | cac100cag | | | |
| TetRrev6-37-1 | 326 | ggg96agg | gca97tca | gtg99ctg | | | |
| TetRrev6-38 | 327 | tac93cac | ggg96tgg | aaa98caa | gtg99ggg | | |
| TetRrev6-39 | 328 | ggg96cag | ggc102agc | | | | |
| TetRrev6-40 | 329 | tac93aac | ggg96tgg | gtg99ggg | | | |
| TetRrev6-50 | 330 | gtg99ggg | | | | | |
| TetRrev6-51 | 331 | gca97aca | gtg99ggg | | | | |
| TetRrev6-53 | 332 | gca97cct | aaa98aga | gtg99gac | | | |
| TetRrev6-54 | 333 | tac93ttc | gac95aac | gtg99ttc | | | |
| TetRrev4/6-3 | 334 | gac95gcc | gtg99ggg | | | | |
| TetRrev4/6-4 | 335 | gac95gcc | gtg99ggg | gtg57gtt | gcg61ggg | | |
| TetRrev4/6-5 | 336 | gcg54tcg | gtg99cgg | | | | |
| TetRrev4/6-6 | 337 | gat53tat | gtg57gtt | gcg61cca | | | |
| TetRrev4/6-7 | 338 | ctg55ctt | ttg60gtg | gca97gta | gtg99ggg | | |
| TetRrev4/6-10 | 339 | gcg56tcg | gtg99gag | | | | |
| TetRrev4/6-15 | 340 | gcg56tcg | atc59gcc | ttg60tgg | gtg99gta | | |
| TetRrev4/6-17 | 341 | ggg99cgg | gtg99ctg | ctc101atc | | | |
| TetRrev4/6-24 | 342 | gcg61acg | gtg99cgg | | | | |
| TetRrev4/6-25 | 343 | atc59agc | ttg60ctg | cgt62ggt | gac95gcc | gtg99ttg | |
| TetRrev4/6-27 | 344 | gtg99cgg | | | | | |
| TetRrev1/34: | 345 | ctt17tct | gaa23aaa | | | | |
| TetRrev3/38 | 346 | ctt17cat | atc22ttc | gaa23gga | | | |
| TetRrev19/48 | 347 | aat18aag | gag19gac | gtc20ctc | atc22aac | gaa23gca | |
| TetRrev22/5 | 348 | gag15gtg | ctt17gct | gag19gat | | | |
| TetRrev25/43 | 349 | ctt17ttt | gag19--- | ggt24ggg | | | |
| TetRrev28/8 | 350 | tta14tca | aat18tat | atc22ttc | tta25ttg | | |
| TetRrev28/16 | 351 | tta14ttg | gag15gcg | ctt17ggt | tta25gta | | |
| TetRrev28/23 | 352 | gca13gcc | tta14tca | aat18aaa | gaa23gta | | |
| TetRrev28/26 | 353 | tta14aca | aat18tat | gga21ggc | atc22ttc | | |
| TetRrev28/27 | 354 | ctt17ggt | atc22atg | | | | |
| TetRrev28/30 | 355 | gtc20ggc | gga21gca | atc22att | gaa23gga | | |
| TetRrev28/31 | 356 | gca13gct | gtc20ggc | gga21cga | atc22aac | ggt24gga | |
| TetRrev28/36 | 357 | aat18ata | gag19ggg | gtc20ggc | gaa23gtc | | |
| TetRrev28/40 | 358 | tta14tca | aat18tat | atc22ttc | tta25ttg | | |
| TetRrev28/41 | 359 | ctt17ctc | aat18gat | gtc20cgc | atc22aac | | |
| TetRrev28/46 | 360 | gca13gct | gtc20ggc | gga21cga | atc22aac | ggt24gga | |
| TetRrev28/48 | 361 | aat18tat | gtc20gac | atc22acc | tgg43tcg | | |
| TetRrev28/49 | 362 | aat18tat | gtc20gac | atc22acc | | | |
| TetRrev29/9 | 363 | ctt17gtt | aat18tat | gga21ggg | atc22acc | | |
| TetRrev29/17 | 364 | ctt17tcc | gaa23gat | | | | |
| TetRrev29/24 | 365 | gag15gtg | ctt17ttt | aat18tat | atc22atg | gaa23aaa | |
| TetRrev29/25 | 366 | gga21--- | atc22--- | gaa23--- | ggt24tcg | | |
| TetRrev29/27 | 367 | tta14gaa | gtc20ggc | | | | |
| TetRrev29/35 | 368 | ctt17tct | gaa23gac | | | | |
| TetRrev29/42 | 369 | gag19gat | ggg96agg | | | | |
| TetRrev29/44 | 370 | gca13gct | gtc20ggc | gga21cga | atc22aac | ggt24gga | |
| TetRrev29/52 | 371 | tta14gta | ctt17gtt | aat18aaa | gtc20gta | ggt24gga | |
| TetRrevAD1/2 | 372 | aat18tat | ctg52ctc | gat53tat | gcg54gcc | atc59acc | |
| TetRrevAD1/6 | 373 | aat18tat | gaa23gta | gat53gct | gcg54tcg | ctg55ctt | gcg56tcg |
| | | gcg61acg | cat63tac | | | | |
| TetRrevAF1/7 | 374 | ggg96agg | ctc101atc | | | | |
| TetRrevAF1/8 | 375 | ggt24ggc | tac93aac | ctc101ttc | ggc102gac | | |
| TetRrevAF1/11 | 376 | tta14ata | gtc20gta | atc22ctc | cgt94cat | gca97gcg | gtg99gag |
| TetRrevAF2/5 | 377 | tta14cga | gag19gaa | gac95cac | ctc101ccc | | |
| TetRrevAD2/4 | 378 | ctt17ttt | aat18cgt | gat53aat | atc59atg | gcg61acg | |
| TetRrevAD2/6 | 379 | aat18tat | gga21gaa | gaa23aaa | | | |
| TetRrevAD2/12 | 380 | gaa23cac | gat53tat | gcg56gcc | gag58cag | atc59acc | |
| TetRrevAD2/13 | 381 | gag19gat | gtc20gaa | gaa23aaa | gtg57ctg | atc59aac | ttg60ttc |
| | | gcg61gct | | | | | |
| TetRrevAD2/2 | 382 | tta14ata | gga21aga | gcg56gtg | | | |
| TetRrevAF1/3 | 383 | ggt24ggg | gac95gcc | ggg96agg | | | |
| TetRrevAF1/4 | 384 | aat18aaa | gac95gtc | ggg96gtg | gca97gta | | |
| TetRrevAF1/5 | 385 | tta14gta | ggt24ggc | tac93gac | cgt94ggt | gac95ggc | |
| TetRrevAD3/2 | 386 | gcg56ccg | gag58cat | gcg61gct | cat63tac | | |
| TetRrevAD3/3 | 387 | gga21--- | atc22--- | gaa23--- | ggt24agt | | |
| TetRrevAF2/7 | 388 | tac93tgc | gac95gaa | gca97aca | | | |
| TetRrevAF6/12 | 389 | tta14ttt | gag15gaa | ggg96gtg | aaa98gaa | gtg99cta | ctc101ccc |
| TetRrevAF7/1 | 390 | tac93tgc | ggg96cgg | aaa98aat | | | |
| TetRrevAF7/2 | 391 | tta14gta | gag15gtg | gaa23gat | aag48aaa | ggg96ctg | ggc102gcc |
| TetRrevAD3/5 | 392 | ctt17cta | aat18aag | gag19gtg | atc22acc | gat53tat | gcg54tcg |
| TetRrevAD3/6 | 393 | gtc20ggc | gcg56ccg | atc59ctc | cgt62cgc | | |
| TetRrevAD3/7 | 394 | ctt17atg | gag19gat | ctg52cta | gat53tac | gcg54gct | gcg56gag |
| TetRrevAD3/8 | 395 | ctg16ctt | gcg56gct | gtg57gag | gag58tta | atc59aac | ttg60gct |
| TetRrevAF2/14 | 396 | tta14ttg | gag15gcg | ctg16ttg | ctt17gtt | aat18aac | gtc20gtt |
| | | ggg96gta | | | | | |
| TetRrevAF2/15 | 397 | gtc20ggc | atc22aac | cgg49cag | gcc50acc | | |
| TetRrevAF2/16 | 398 | gag19gtg | tac93tgc | gac95tac | gca97aca | aaa98aac | gtg99ctg |
| | | ctc101ctg | | | | | |
| TetRrevAF3/5 | 399 | gtc20ttc | gga21aga | cgg49agt | | | |
| TetRrevAD3/9 | 400 | gag15cag | ctt17ctc | ctg55atg | atc59agg | | |
| TetRrevAD3/10 | 401 | aat18tat | ggt24ggc | cta51ctc | gat53tac | gcg54gtg | |
| TetRrevAD3/11 | 402 | tta14gta | ggt24ggc | tac93gac | cgt94ggt | gac95ggc | |
| TetRrevAF3/6 | 403 | gaa23aaa | gac95cac | ctc101cac | | | |
| TetRrevAF3/7 | 404 | ctt17cat | atc22ttc | gca97cca | | | |
| TetRrevAF3/8 | 405 | gag15gtg | ctg16ctc | aat18cat | gag19gat | gtc20gac | cgt94ccc |
| | | gac95cac | cac100aac | | | | |
| TetRrevAF3/10 | 406 | ggg96ggt | gtg99ggg | cac100ccc | | | |
| TetRrevAF4/1 | 407 | gag15gtg | gtc20gcc | atc22ttc | gaa23cag | tta25ttg | gca96gta |
| | | aaa98gaa | ggc102gga | | | | |
| TetRrevAF4/3 | 408 | ctg16cag | aat18cat | gag19gtg | gtc20ggt | atc22ata | ggg96acg |
| TetRrevAF4/4 | 409 | ggt24gca | cgt94cct | gac95gaa | ggc102gac | | |
| TetRrevAF4/5 | 410 | gag15ggg | ctg16ttg | gac95gcc | gtg99ggg | | |
| TetRrevAF4/6 | 411 | gag15gac | ctt17ata | aat18act | gaa23gga | gcg54gtg | gac95gcc |
| TetRrevAF4/7 | 412 | tta14gta | gca97aca | gtg99ggg | | | |
| TetRrevAF4/8 | 413 | tta14ttc | gaa23gca | ggg96atg | gca97cca | ctc101ccc | |
| TetRrevAF4/9 | 414 | ctg16cgg | gag19gat | gaa23gat | gac95ggc | gtg99gcg | ggc102ggg |
| TetRrevAD2/5 | 415 | ctt17ttt | ctg55ctt | ttg60ttc | cgt62gtg | | |
| TetRrevAD2/8 | 416 | gtc20gga | gat53aat | gcg54gca | gcg56ggg | gtg57ctg | atc59ttc |
| | | ttg60ttc | cgt62cga | | | | |
| TetRrevAD2/1 | 417 | gga21ggg | cta51ctc | gat53tat | ctg55cta | gcg56ccg | gcg61gag |
| TetRrevAF5/10 | 418 | cgt94cct | gg96gtg | gca97aca | aaa98aac | cac100cag | |
| TetRrevAF4/12 | 419 | ctg16cag | aat18tat | gca97gga | cac100tcc | | |
| TetRrevAF4/13 | 420 | gca13gcc | gag15gat | ctt17ctg | gag19gtg | atc22ata | gaa23aaa |
| | | cgt94cat | gac95aac | | | | |
| TetRrevAF5/1 | 421 | tta14ttt | gac95gcc | gtg99ggg | | | |
| TetRrevAF5/3 | 422 | gag15ggg | aat18aag | cgt94cat | ggg96ggc | ggc102gtc | |
| TetRrevAF5/5 | 423 | tta14gta | ctt17ttt | gtg20gcc | atc22atg | tta25ttt | gac95cac |
| TetRrevAF5/6 | 424 | aat18cat | tta25ttt | gca97cca | aaa98aac | ctc101cac | |
| TetRrevAF5/7 | 425 | gag15gcg | ggg96atg | gca97cca | ctc101ccc | | |
| TetRrevAF5/8 | 426 | atc22ata | cgt94cct | gtg99gag | | | |
| TetRrevAF5/9 | 427 | cgt94tgt | gtg99gag | | | | |
| TetRrevAF5/11 | 428 | aat18aag | gag19gcg | ctc101cta | ggc102agc | | |
| TetRrevAF5/13 | 429 | tta14cta | gtc20ggc | gtg99gag | | | |
| TetRrevAF6/1 | 430 | gca13gcg | gag19gtg | gtc20gcc | gac95atc | ggg96gga | |
| TetRrevAF6/2 | 431 | aat18gat | gaa23gca | gac95aac | cac100ccc | ctc101tcc | |
| TetRrevAF6/3 | 432 | ggg96ggt | gtg99ggg | cac100ccc | | | |
| TetRrevAF6/4 | 433 | tac93tat | ggg96cgg | gca97cca | | | |
| TetRrevAF6/5 | 434 | gtc20gta | atc22gtc | gaa23gat | gac95aac | cac100ccc | |
| TetRrevAF6/6 | 435 | gac95tac | ggg96gag | gtg99gtc | | | |
| TetRrevAF6/7 | 436 | ggg96ggt | gtg99ggg | cac100ccc | | | |
| TetRrevAF6/8 | 437 | gcg71acg | ggg96gag | | | | |
| TetRrev96/99-1 | 438 | ggg96cac | gtg99agg | | | | |
| TetRrev96/99-2 | 439 | ggg96aag | gtg99gcc | | | | |
| TetRrev96/99-3 | 440 | ggg96gag | gtg99acc | | | | |
| TetRrev96/99-4 | 441 | ggg96ccc | gtg99tcg | | | | |
| TetRrev96/99-5 | 442 | ggg96atc | gtg99aag | | | | |
| TetRrev96/99-6 | 443 | ggg96aac | gtg99cag | | | | |
| TetRrev96/99-7 | 444 | ggg96ctg | gtg99aag | | | | |
| TetRrev96/99-8 | 445 | ggg96aac | gtg99cac | | | | |
| TetRrev96/99-9 | 446 | ggg96cac | gtg99aac | | | | |
| TetRrev96/99-10 | 447 | ggg96aac | gtg99ccg | | | | |
| TetRrev96/99-11 | 448 | ggg96agg | gtg99tac | | | | |
| TetRrev96/99-12 | 449 | ggg96cac | gtg99cag | | | | |
| TetRrev96/99-13 | 450 | ggg96acc | gtg99gac | | | | |
| TetRrev96/99-14 | 451 | ggg96aac | gtg99aac | | | | |
| TetRrev96/99-15 | 452 | ggg96ccg | gtg99ccc | | | | |
| TetRrev96/99-16 | 453 | ggg96ccc | gtg99tac | | | | |
| TetRrev96/99-17 | 454 | ggg96acc | gtg99aag | | | | |
| TetRrev96/99-18 | 455 | ggg96acc | gtg99ccc | | | | |
| TetRrev96/99-19 | 456 | ggg96cgt | gtg99tcg | | | | |
| TetRrev96/99-20 | 457 | ggg96tcc | gtg99aag | | | | |
| TetRrev96P | 458 | ggg96ccc | | | | | |

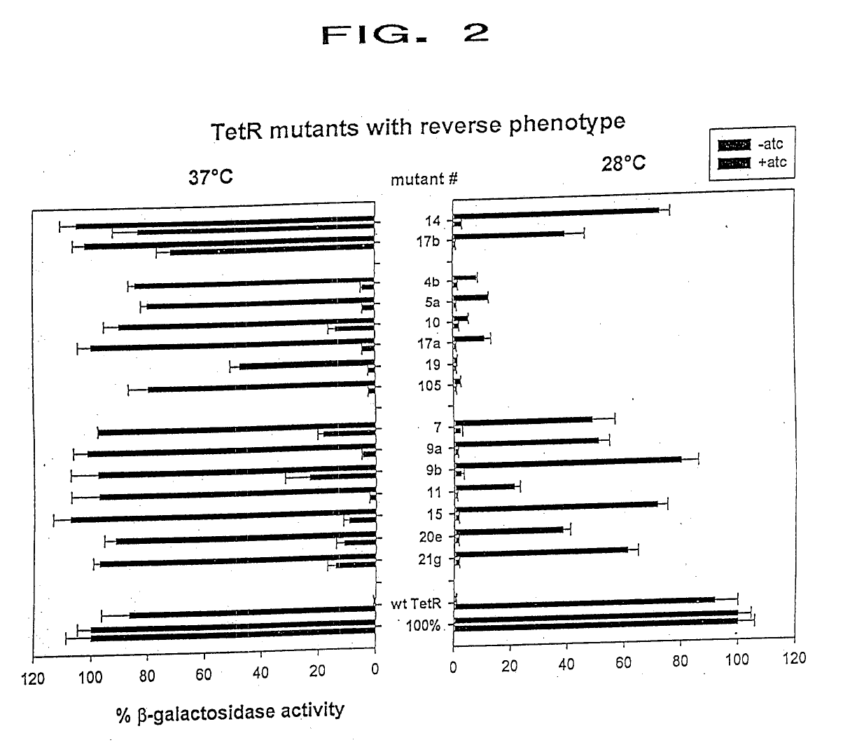

In one specific embodiment, modified revTetR repressors of the present invention comprise an amino acid substitution of arginine for glycine at position 96 (e.g., SEQ ID NO. 24). Additional modified revTetR repressors of the present invention comprise the arginine for glycine substitution at position 96 and further comprise a substitution or substitutions of serine for threonine at position 103 and valine for glutamic acid at position 114 (e.g., SEQ ID NO. 2); leucine for proline at position 159 (e.g., SEQ ID NO. 6); glutamine to histidine at position 188 (e.g., SEQ ID NO. 12). Interestingly, as described below, each of the revTetR repressor has a different activity compared to the others demonstrating that each substitution or combination of substitutions contributes to or modulates the activity of the resulting revTetR repressor protein and that the activity is not solely derived from the single arginine substitution at position 96 (e.g., see FIG. 2).

In another embodiment, modified revTetR repressors of the present invention comprise an amino acid substitution of glutamic acid for glycine at position 96 and further comprise a substitution or substitutions of aspargine for aspartic acid at position 157 and histidine for glutamine at position 200 (e.g., SEQ ID NO. 4); serine for leucine at position 205 (e.g., SEQ ID NO. 14); or phenylalanine for tryptophan at position 110 (e.g., SEQ ID NO. 16). Similar to the G96R substitutions above, each of the revTetR proteins has a different activity compared to each other demonstrating that each substitution or combination of substitutions contributes to or modulates the activity of the resulting revTetR repressor protein and that the observed activity is not solely derived from the single glutamic acid substitution at position 96 (e.g., see FIG. 2).

In yet another embodiment, modified revTetR repressors of the present invention comprise an amino acid substitution of glutamic acid for valine at position 99 (SEQ ID NO. 26). Additional modified revTetR repressors of the present invention comprise glutamic acid for valine at position 99 and further comprise a substitution or substitutions of valine for isoleucine at position 194 (e.g., SEQ ID NO. 18); cysteine for arginine at position 158 (e.g., SEQ ID NO. 20); or valine for alanine at position 70 and glutamine for leucine at position 91 (e.g., SEQ ID NO. 22). Similarly to the G96R and G96E class of revTetR repressors, each of the V99E-substituted revTetR protein has a different activity compared to each other demonstrating that each substitution or combination of substitutions contributes to or modulates the activity of the resulting revTetR repressor protein and that the observed activity is not solely derived from the single valine substitution at position 99 (e.g., see FIG. 2).

Furthermore, modified revTetR repressors of the present invention comprise an amino acid substitution of asparagine for isoleucine for position 59, glutamic acid for aspartic acid at position 95, and alanine for histidine at position 100 (e.g., SEQ ID NO. 10); asparagine for leucine at position 59, arginine for lysine at position 98, histidine for leucine at position 101 and glycine for serine at position 192 (e.g., SEQ ID NO. 30); valine for alanine at position 160, valine for aspartic acid at position 178, tryptophan for glycine at position 196 (e.g., SEQ ID NO. 8); and, valine for alanine at position 71, glycine (GGC) for aspartic acid at position 95, and arginine for leucine at position 127 (e.g., SEQ ID NO. 28).

In other preferred embodiments, the purified revTetR repressors of the present invention comprise any of the amino acid sequences set forth in SEQ ID NOS. 2, 4, 6, 8, 10, 12, 14, 16, 18, 20, 22, 24, 26, 28, 30, and 71-264.

In addition, the methods and compositions of the invention also use and encompass proteins and polypeptides that represent functionally equivalent gene products. Such functionally equivalent gene products include, but are not limited to, natural variants of the polypeptides having an amino acid sequence set forth in SEQ ID NO: 2, 4, 6, 8, 10, 12, 14, 16, 18, 20, 22, 24, 26, 28, 30, and 71-264. Such equivalent revTetR repressors can contain, e.g., deletions, additions or substitutions of amino acid residues within the amino acid sequences encoded by the target gene sequences described above, but which result in a silent change, thus producing a functionally equivalent revTetR repressor product. As described above, nucleotide substitutions in the coding region of revTetR repressors that did not result in a corresponding codon change were identified using the cell-based assay in Section 5.5.2.

Amino acid substitutions can be made on the basis of similarity in polarity, charge, solubility, hydrophobicity, hydrophilicity size, nucleophilicity, and/or the amphipathic nature of the residues involved. Examples of such classifications, some of which overlap include, nonpolar (i.e., hydrophobic) amino acid residues can include alanine (Ala or A), leucine (Leu or L), isoleucine (Ile or I), valine (Val or V), proline (Pro or P), phenylalanine (Phe or F), tryptophan (Trp or W) and methionine (Met or M); polar neutral amino acid residues can include glycine (Gly or G), serine (Ser or S), threonine (Thr or T), cysteine (Cys or C), tyrosine (Tyr or Y), asparagine (Asn or N) and glutamine (Gln or Q); small amino acids include glycine (Gly or G), and alanine (Ala or A); hydrophobic amino acid residues can include valine (Val or V), leucine (Leu or L), isoleucine (Ile or I), methionine (Met or M), and proline (Pro or P); nucleophilic amino acids can include serine (Ser or S), threonine (Thr or T), and cysteine (Cys or C); aromatic amino acids can include phenylalanine (Phe or F), tyrosine (Tyr or Y), and tryptophan (Trp or W); amide amino acids can include asparagine (Asn or N), and glutamine (Gln or Q); positively charged (i.e., basic) amino acid residues can include arginine (Arg or R), lysine (Lys or K) and histidine (His or H); and negatively charged (i.e., acidic) amino acid residues can include aspartic acid (Asp or D) and glutamic acid (Glu or E). Thus, other amino acid substitutions, deletions or additions at these or other amino acid positions that retain the desired functional properties of the revTetR repressors are within the scope of the invention.

5.2.3 Modified Repressors of Other Classes

In a further embodiment of the present invention, a non-chimeric Tet repressor selected from the TetR(A), TetR(B), TetR(C), TetR(D), TetR(E), TetR(G), TetR(H), TetR(J), and TetR(Z) classes of TetR repressor proteins, is expressed from a mutated coding sequence encoding one or more of amino acid substitutions to provide a modified TetR protein which binds to DNA with greater affinity in the presence of tetracycline or a tetracycline analog than in the absence of tetracycline or tetracycline analog, i.e. a revTet repressor.

In certain embodiments, a TetR protein of the present invention is a non-chimeric TetR repressor protein selected from the TetR(A), TetR(B), TetR(C), TetR(D), TetR(E), TetR(G), TetR(H), TetR(J), and TetR(Z) classes of TetR repressor proteins and which comprises at least one amino acid substitution at a position corresponding to the following amino acid position and/or positions of the tetR(BD) chimera depicted in SEQ ID NO: 32, position 96 or 99; positions 96, 103 and 114; positions 96, 157 and 200; positions 96 and 159; positions 160, 178, 196; positions 59, 95 and 100; positions 96 and 188; positions 96 and,205; positions 96 and 110; positions 99 and 194; positions 99 and 158; positions 70, 91 and 99; positions 71, 95 and 127; positions 59, 98, 101 and 192.

In certain embodiments, the amino acid substitutions at these positions (with respect to the amino acid sequence depicted in SEQ ID NO: 32) that confer a reverse phenotype in prokaryotes include, but are not limited to, Asn at position 59, Val at positions 70 and 71; Gln at position 91; Glu and Gly at position 95; Arg and Glu at position 96; Arg at position 98; Glu at position 99; Ala at position 100; His at position 101; Ser at position 103; Phe at position 110; Val at position 114; Arg at position 127; Asn at position 157; Cys at position 158; Leu at position 159; Gln at position 188; Gly at position 192; Val at position 194; Trp at position 196; His at position 200; and Ser at position 205.

In specific embodiments, a TetR protein selected from any of the TetR(A), TetR(B), TetR(C), TetR(D), TetR(E), TetR(G), TetR(H), TetR(J), and TetR(Z) classes of TetR repressor proteins, is modified to provide a revTetR repressor of the present invention that comprises arginine at the amino acid corresponding to the amino acid at position 96 of SEQ ID NO: 32.

In other specific embodiments, a TetR protein selected from any of the TetR(A), TetR(B), TetR(C), TetR(D), TetR(E), TetR(G), TetR(H), TetR(J), and TetR(Z) classes of TetR repressor proteins, is modified to provide a revTetR repressor of the present invention that comprises a glycine residue at the amino acid position corresponding to amino acid position 96 of SEQ ID NO: 32, and/or comprises serine at position 103, valine at position 114; leucine at position 159; and glutamine at position 188, where each amino acid position corresponds to the amino acid position of the protein sequence depicted in SEQ ID NO: 32.

In another embodiment, a TetR repressor protein selected from the TetR(A), TetR(B), TetR(C), TetR(D), TetR(E), TetR(G), TetR(H), TetR(J), and TetR(Z) classes of TetR repressor proteins is modified to provide a revTetR repressor of the present invention that comprises glutamic acid at position 96 and further at position 157 and histidine at position 200; serine at position 205; or phenylalanine at position 110, where each amino acid position corresponds to the amino acid position of the protein sequence depicted in SEQ ID NO: 32.

In yet another embodiment, a TetR repressor protein selected from the TetR(A), TetR(B), TetR(C), TetR(D), TetR(E), TetR(G), TetR(H), TetR(J), and TetR(Z) classes of TetR repressor proteins is modified to provide a modified revTetR repressor of the present invention that comprises glutamic acid at position 99; glutamic acid at position 99 and valine at position 194; cysteine at position 158; valine at position 70 and glutamine at position 91; asparagine at position 59, glutamic acid at position 95, and alanine at position 100; asparagine at position 59, arginine at position 98, histidine at position 101 and glycine at position 192; valine at position 160, valine at position 178, tryptophan at position 196; and, valine at position 71, glycine at position 95, and arginine at position 127; where each amino acid position corresponds to the amino acid position of the protein sequence depicted in SEQ ID NO: 32.

Such non-chimeric revTetR repressor proteins of the present invention constructed from any TetR repressor protein of the TetR(A), TetR(B), TetR(C), TetR(D), TetR(E), TetR(G), TetR(H), TetR(J), and TetR(Z) classes, also, therefore include all members of these classes of TetR proteins and is not to be limited to the specific, exemplary proteins provided in SEQ ID NO: 34, 36, 38, 40, 42, 44, 46, 48, 50 that correspond, respectively to the nine TetR classes provided, and are encoded, respectively by the nucleotide sequence provided in SEQ ID NO: 33, 35, 37, 39, 41, 43, 45, 47, and 49. Moreover, the revTetR repressor proteins of the present invention constructed from any TetR repressor of classes A, B, C, D, E, G, H, J, and Z, also encompass proteins and polypeptides that represent functionally equivalent gene products, including, but not limited to, natural variants of these polypeptides having an amino acid sequence set forth in SEQ ID NO: 32, 34, 36, 38, 40, 42, 44, 46, 48, and 50. Such equivalent revTetR repressors can also contain, e.g., deletions, additions or substitutions of amino acid residues within the amino acid sequences encoded by the target gene sequences described above, but which result in a silent change, thus producing a functionally equivalent revTetR repressor product.

For example, amino acid substitutions can be made on the basis of similarity in polarity, charge, solubility, hydrophobicity, hydrophilicity size, nucleophilicity, and/or the amphipathic nature of the residues involved. Examples of such classifications, some of which overlap include, nonpolar (i.e., hydrophobic) amino acid residues can include alanine (Ala or A), leucine (Leu or L), isoleucine (Ile or I), valine (Val or V), proline (Pro or P), phenylalanine (Phe or F), tryptophan (Trp or W) and methionine (Met or M); polar neutral amino acid residues can include glycine (Gly or G), serine (Ser or S), threonine (Thr or T), cysteine (Cys or C), tyrosine (Tyr or Y), asparagine (Asn or N) and glutamine (Gln or Q); small amino acids include glycine (Gly or G), and alanine (Ala or A); hydrophobic amino acid residues can include valine (Val or V), leucine (Leu or L), isoleucine (Ile or I), methionine (Met or M), and proline (Pro or P); nucleophilic amino acids can include serine (Ser or S), threonine (Thr or T), and cysteine (Cys or C); aromatic amino acids can include phenylalanine (Phe or F), tyrosine (Tyr or Y), and tryptophan (Trp or W); amide amino acids can include asparagine (Asn or N), and glutamine (Gln or Q); positively charged (i.e., basic) amino acid residues can include arginine (Arg or R), lysine (Lys or K) and histidine (His or H); and negatively charged (i.e., acidic) amino acid residues can include aspartic acid (Asp or D) and glutamic acid (Glu or E). Thus, other amino acid substitutions, deletions or additions at these or other amino acid positions that retain the desired functional properties of the revTetR repressors are within the scope of the invention.

In other embodiments of the present invention, the specific amino acid substitutions identified as described herein with TetR(BD) chimeras, may also, in turn, be substituted by similar, functionally equivalent amino acids, i.e. those indicated in the preceding paragraph, to provide additional revTetR repressors that are within the scope of the invention. That is, a revTetR repressor protein of the present invention can be constructed from any TetR repressor protein of the TetR(A), TetR(B), TetR(C), TetR(D), TetR(E), TetR(G), TetR(H), TetR(J), and TetR(Z) classes by substituting, at the position corresponding to that identified in the TetR(BD) chimera depicted in SEQ ID NO: 32, either the exact amino acid identified in the revTet(BD) chimeras depicted in SEQ ID NO: 2, 4, 6, 8, 10, 12, 14, 16, 18, 20, 22, 24, 26, 28, 30, and 71-264, or, in certain embodiments, the functional equivalent of that amino acid.

The amino acid substitutions of the present invention and their functional equivalents can be introduced into TetR proteins of each of the nine classes of TetR proteins, to provide novel revTet repressor proteins. The position of each of the amino acid substitutions disclosed above is numbered according to the amino acid sequence of the TetR(BD) chimeric protein of SEQ ID NO: 32. As would be apparent to one of ordinary skill, the corresponding amino acid to be substituted in another TetR protein such as, but not limited to those members of the TetR(A), TetR(B), TetR(C), TetR(D), TetR(E), TetR(G), TetR(H), TetR(J), and TetR(Z) classes of TetR repressor proteins to provide a revTetR protein, is readily identified using methods and tools well known in the art. For example, the amino acid sequence of a subject TetR repressor is readily compared with that provided by SEQ ID NO: 32 using software publically available from the National Center for Biotechnology Information and the National Library of Medicine at http://www.ncbi.nlm.nih.gov/BLAST. (For a description of this software, see Tatusova et al. (1999) FEMS Microbiol Lett 177(1): 187-88).

For example, comparisons have been carried out for each representative TetR(A), TetR(B), TetR(C), TetR(D), TetR(E), TetR(G), TetR(H), TetR(J), and TetR(Z) protein disclosed above, to provide the position and nature of the amino acid corresponding to each of the substitutions disclosed herein, for each representative class member. The results of such comparisons are summarized in Table 3, where TetR(BD) is SEQ ID NO: 32, TetR(A) is SEQ ID NO: 34, TetR(B) is SEQ ID NO: 36, TetR(C) is SEQ ID NO: 38, TetR(D) is SEQ ID NO: 40, TetR(E) is SEQ ID NO: 42, TetR(G) is SEQ ID NO: 44, TetR(H) is SEQ ID NO: 46, TetR(J) is SEQ ID NO: 48, and TetR(Z) is SEQ ID NO: 50. The first column of Table 3 provides the wild type amino acid residue, the amino acid position number, and the substituted amino acid residue found at that position in the revTet(BD) mutants disclosed above. The corresponding amino acid position and wild type amino acid residue for each representative member of TetR A, B, C, D, E, G, H, J, and Z are provided in the remaining nine columns of Table 3.

TABLE 3

| TetR(BD) | | | | | | | | | |
|---|---|---|---|---|---|---|---|---|---|
| revTet | TetR | TetR | TetR | TetR | TetR | TetR | TetR | TetR | TetR |
| allele | (A) | (B) | (C) | (D) | (E) | (G) | (H) | (J) | (Z) |
| I 59 N | M 59 | M 59 | M 59 | I 59 | I 59 | M 59 | I 59 | I 59 | V 63 |
| A 70 V | R 70 | L 70 | P 70 | A 70 | L 70 | E 70 | L 70 | L 70 | E 74 |
| A 71 V | A 71 | E 71 | D 71 | A 71 | P 71 | E 71 | P 71 | A 71 | S 75 |
| L 91 Q | L 91 | L 91 | L 91 | L 91 | L 91 | L 91 | L 91 | L 91 | H 95 |
| D 95 E | D 95 | D 95 | D 95 | D 95 | D 95 | D 95 | D 95 | D 95 | D 99 |
| D 95 G | | | | | | | | | |
| G 96 R | G 96 | G 96 | G 96 | G 96 | G 96 | G 96 | G 96 | G 96 | G 100 |
| G 96 E | | | | | | | | | |
| K 98 R | R 98 | K 98 | R 98 | K 98 | R 98 | R 98 | K 98 | K 98 | R 102 |
| V 99 E | I 99 | V 99 | I 99 | V 99 | L 99 | I 99 | I 99 | I 99 | L 103 |
| H 100 A | H 100 | H 100 | H 100 | H 100 | H 100 | H 100 | H 100 | H 100 | H 104 |
| L 101 H | A 101 | L 101 | A 101 | L 101 | I 101 | A 101 | A 101 | A 101 | A 105 |
| T 103 S | T 103 | T 103 | T 103 | T 103 | T 103 | T 103 | T 103 | T 103 | H 107 |
| Y 110 F | M 110 | Y 110 | M 110 | Y 110 | F 110 | F 110 | F 110 | F 110 | D 114 |
| E 114 V | D 114 | E 114 | D 114 | E 114 | E 114 | E 114 | E 114 | E 114 | E 118 |
| L 127 R | A 127 | L 127 | A 127 | L 127 | V 127 | P 127 | L 127 | L 127 | E 137 |
| D 157 N | E 157 | E 157 | E 157 | D 157 | N 157 | D 159 | E 157 | E 157 | G 164 |
| R 158 C | R 158 | R 158 | R 158 | R 158 | H 158 | R 160 | R 158 | R 158 | N 165 |
| P 159 L | G 159 | E 159 | G 159 | P 159 | V 159 | P 161 | E 159 | E 159 | A 166 |
| A 160 V | G 160 | T 160 | T 164 | A 160 | I 160 | D 162 | K 160 | K 160 | S 167 |
| D 178 V | D 179 | D 178 | Y 182 | D 178 | A 175 | E 180 | D 180 | D 180 | — |
| H 188 Q | Q 189 | F 188 | R 192 | H 188 | F 185 | F 190 | F 190 | F 190 | F 177 |
| S 192 G | V 193 | L 192 | L 196 | S 192 | S 189 | S 194 | V 194 | V 194 | A 181 |
| I 194 V | V 195 | I 194 | I 198 | I 194 | I 191 | I 196 | I 196 | I 196 | I 183 |
| G 196 W | G 197 | G 196 | G 200 | G 196 | G 193 | G 198 | G 198 | G 198 | G 185 |
| Q 200 H | R 201 | Q 200 | M 204 | Q 200 | Q 197 | L 202 | V 202 | V 202 | S 189 |
| L 205 S | N 206 | S 205 | N 209 | L 205 | K 202 | L 207 | K 207 | H 207 | L 194 |

In light of the demonstrated sequence conservation between and among the TetR repressor proteins previously characterized, such an analysis can be performed with any TetR repressor protein including, but not limited to, other known members of these nine classes of TetR proteins. For instance, based on the information provided in Table 3, one of skill in the art can introduce the same substitution or substitutions as provided for TetR(BD) into any one of the listed TetR repressor class for the amino acid positions 91, 95, 96, 100, 103 and 196.

Furthermore, the amino acid substitution identified at position 114 involved in the reverse phenotype was valine for glutamic acid. While glutamic acid is present in TetR classes B and E, the amino acid at position 114 in TetR classes A and C is an aspartic acid, also a negatively charged amino acid residue. Therefore, replacement of the aspartic acid codon with a codon for a hydrophobic amino acid, such as valine, would be predicted to have similar functional result in these classes. Similar substitutions may be introduced at other positions to generate isolated nucleic acids of the present invention.

Therefore, once the corresponding amino acid(s) have been identified, they, or their functional equivalents can be introduced into another TetR protein, or tetracycline-binding domain thereof, of each of the nine classes of TetR proteins, to provide a novel revTet repressor protein, using recombinant DNA techniques that are disclosed below and that are well known in the art. Accordingly, in another embodiment, the present invention is directed toward chimeric tetracycline repressor proteins that comprise, for example, a tetracycline-binding domain derived from a revTetR protein of any of the TetR(A), TetR(B), TetR(C), TetR(D), TetR(E), TetR(G), TetR(H), TetR(J), and TetR(Z) classes of TetR binding proteins as disclosed above, that is operatively associated with a DNA-binding domain, which may be derived from another TetR repressor protein or from a non-TetR repressor, DNA-binding protein. In this embodiment, the tetracycline-binding domain carries one or more of the amino acid substitutions disclosed above such that the modified chimeric revTetR protein binds to DNA with greater affinity in the presence of tetracycline or a tetracycline analog than it does in the absence of tetracycline or a tetracycline analog.

As used herein, the term “DNA-binding domain” generally encompasses, for example, approximately the first 50 amino-terminal residues of each TetR protein, which includes the helix-turn-helix structural motif known to be involved in the DNA recognition and binding.

As used herein, the term “tetracycline-binding domain” is generally intended to encompass that portion of a TetR protein other than the amino-terminal DNA-binding domain, and therefore, includes not only the tetracycline-binding portion but also those portions of the Tet repressor molecule that may be required for dimer formation. In other aspects of this embodiment, the tetracycline-binding domain of a chimeric revTetR protein comprises the carboxy terminal part of the polypeptide.

In certain embodiments, the chimeric revTetR proteins of the present invention consist essentially of from about 180 to about 230 amino acids, from about 185 to about 225 amino acids, from about 190 amino acids to about 220 amino acids, and from about 195 amino acids to about 215 amino acids.

In one embodiment, the present invention is directed toward a modified TetR(A) protein comprising an amino acid substitution at a position selected from the group consisting of positions 59, 70, 71, 91, 95, 96, 99, 100, 101, 103, 114, 127, 158, 159, 160, 179, 193, 197, and 201 of the TetR(A) protein as depicted in SEQ ID NO: 34, wherein said modified TetR(A) protein binds a TetR(A) operator sequence with greater affinity in the presence of tetracycline than in the absence of tetracycline. In particular aspects of this embodiment: the amino acid substitution at position 59 is selected from the group consisting of glycine, serine, threonine, cysteine, tyrosine, asparagine and glutamine; the amino acid substitution at position 70 is selected from the group consisting of isoleucine, valine, phenylalanine, methionine, and tryptophan; the amino acid substitution at position 71 is selected from the group consisting of leucine, isoleucine, valine, phenylalanine, methionine, and tryptophan; the amino acid substitution at position 91 is selected from the group consisting of glycine, serine, threonine, cysteine, tyrosine, asparagine, and glutamine; the amino acid substitution at position 95 is selected from the group consisting of glycine, serine, threonine, cysteine, tyrosine, asparagine, glutamine, alanine, and glutamic acid; the amino acid substitution at position 96 is selected from the group consisting of aspartic acid, glutamic acid, arginine, lysine, and histidine; the amino acid substitution at position 99 is selected from the group consisting of aspartic acid, and glutamic acid; the amino acid substitution at position 100 is selected from the group consisting of alanine, leucine, isoleucine, valine, proline, phenylalanine, tryptophan, and methionine; the amino acid substitution at position 101 is selected from the group consisting of arginine, lysine, and histidine; the amino acid substitution at position 103 is selected from the group consisting of glycine, serine, cysteine, tyrosine, asparagine, and glutamine; the amino acid substitution at position 114 is selected from the group consisting of alanine, leucine, isoleucine, valine, proline, phenylalanine, tryptophan, and methionine; the amino acid substitution at position 127 is selected from the group consisting of arginine, lysine, and histidine; the amino acid substitution at position 158 is selected from the group consisting of glycine, serine, threonine, cysteine, tyrosine, and glutamine; the amino acid substitution at position 159 is selected from the group consisting of methionine, leucine, isoleucine, phenylalanine, and tryptophan; the amino acid substitution at position 160 is selected from the group consisting of methionine, leucine, valine, proline, phenylalanine, and tryptophan; the amino acid substitution at position 179 is selected from the group consisting of methionine, leucine, isoleucine, valine, proline, phenylalanine, and tryptophan; the amino acid substitution at position 193 is selected from the group consisting of glycine, threonine, cysteine, tyrosine, asparagine, and glutamine; the amino acid substitution at position 197 is selected from the group consisting of alanine, leucine, isoleucine, valine, proline, phenylalanine, tryptophan, and tyrosine; and the amino acid substitution at position 201 is selected from the group consisting of arginine, lysine, and histidine.

In another embodiment, the present invention is directed toward a modified TetR(A) protein comprising an amino acid substitution at a position selected from the group consisting of positions 59, 70, 71, 91, 95, 96, 99, 100, 101, 103, 114, 127, 158, 159, 160, 179, 193, 197, and 201 of the TetR(A) protein as depicted in SEQ ID NO: 34, wherein said modified TetR(A) protein binds a TetR(A) operator sequence with greater affinity in the presence of tetracycline than in the absence of tetracycline; wherein: the amino acid substitution at position 59 is asparagine; the amino acid substitution at position 70 is valine; the amino acid substitution at position 71 is valine; the amino acid substitution at position 91 is glutamine; the amino acid substitution at position 95 is selected from the group consisting of glycine and glutamic acid; the amino acid substitution at position 95 is glycine; the amino acid substitution at position 95 is glutamic acid; the amino acid substitution at position 96 is arginine; the amino acid substitution at position 96 is glutamic acid; the amino acid substitution at position 99 is glutamic acid; the amino acid substitution at position 100 is alanine; the amino acid substitution at position 101 is histidine; the amino acid substitution at position 103 is serine; the amino acid substitution at position 114 is valine; the amino acid substitution at position 127 is selected from the group consisting of arginine, lysine, and histidine; the amino acid substitution at position 158 is cysteine; the amino acid substitution at position 159 is leucine; the amino acid substitution at position 160 is valine; the amino acid substitution at position 179 is valine; the amino acid substitution at position 193 is glycine; the amino acid substitution at position 197 is tryptophan; and the amino acid substitution at position 201 is histidine. In further embodiments, the present invention is directed toward modified TetR(A) proteins that comprise the single or multiple amino acid substitutions at positions of the TetR(A) protein that correspond to those identified in the revTetR(BD) chimeras of Table 1.

In another embodiment, the present invention is directed toward a modified TetR(B) protein comprising an amino acid substitution at a position selected from the group consisting of positions 59, 70, 71, 91, 95, 96, 99, 100, 101, 103, 114, 127, 158, 159, 160, 178, 192, 196, and 200 of the TetR(B) protein as depicted in SEQ ID NO: 36, wherein said modified TetR(B) protein binds a TetR(B) operator sequence with greater affinity in the presence of tetracycline than in the absence of tetracycline. In particular aspects of this embodiment: the amino acid substitution at position 59 is selected from the group consisting of glycine, serine, threonine, cysteine, tyrosine, asparagine and glutamine; the amino acid substitution at position 70 is selected from the group consisting of isoleucine, valine, phenylalanine, methionine, and tryptophan; the amino acid substitution at position 71 is selected from the group consisting of leucine, isoleucine, valine, phenylalanine, methionine, and tryptophan; the amino acid substitution at position 91 is selected from the group consisting of glycine, serine, threonine, cysteine, tyrosine, asparagine, and glutamine; the amino acid substitution at position 95 is selected from the group consisting of glycine, serine, threonine, cysteine, tyrosine, asparagine, glutamine, alanine, and glutamic acid; the amino acid substitution at position 96 is selected from the group consisting of aspartic acid, glutamic acid, arginine, lysine, and histidine; the amino acid substitution at position 99 is selected from the group consisting of aspartic acid, and glutamic acid; the amino acid substitution at position 100 is selected from the group consisting of alanine, leucine, isoleucine, valine, proline, phenylalanine, tryptophan, and methionine; the amino acid substitution at position 101 is selected from the group consisting of arginine, lysine, and histidine; the amino acid substitution at position 103 is selected from the group consisting of glycine, serine, cysteine, tyrosine, asparagine, and glutamine; the amino acid substitution at position 114 is selected from the group:consisting of alanine, leucine, isoleucine, valine, proline, phenylalanine, tryptophan, and methionine; the amino acid substitution at position 127 is selected from the group consisting of arginine, lysine, and histidine; the amino acid substitution at position 158 is selected from the group consisting of glycine, serine, threonine, cysteine, tyrosine, and glutamine; the amino acid substitution at position 159 is selected from the group consisting of methionine, leucine, isoleucine, phenylalanine, and tryptophan; the amino acid substitution at position 160 is selected from the group consisting of methionine, leucine, valine, proline, phenylalanine, and tryptophan; the amino acid substitution at position 178 is selected from the group consisting of methionine, leucine, isoleucine, valine, proline, phenylalanine, and tryptophan; the amino acid substitution at position 192 is selected from the group consisting of glycine, threonine, cysteine, tyrosine, asparagine, and glutamine; the amino acid substitution at position 196 is selected from the group consisting of alanine, leucine, isoleucine, valine, proline, phenylalanine, tryptophan, and tyrosine; and the amino acid substitution at position 200 is selected from the group consisting of arginine, lysine, and histidine.

In another embodiment, the present invention is directed toward a modified TetR(B) protein comprising an amino acid substitution at a position selected from the group consisting of positions 59, 70, 71, 91, 95, 96, 99, 100, 101, 103, 114, 127, 158, 159, 160, 178, 192, 196, and 200 of the TetR(B) protein as depicted in SEQ ID NO: 36, wherein said modified TetR(B) protein binds a TetR(B) operator sequence with greater affinity in the presence of tetracycline than in the absence of tetracycline, wherein: the amino acid substitution at position 59 is asparagine; the amino acid substitution at position 70 is valine; the amino acid substitution at position 71 is valine; the amino acid substitution at position 91 is glutamine; the amino acid substitution at position 95 is selected from the group consisting of glycine and glutamic acid; the amino acid substitution at position 95 is glycine; the amino acid substitution at position 95 is glutamic acid; the amino acid substitution at position 96 is arginine; the amino acid substitution at position 96 is glutamic acid; the amino acid substitution at position 99 is glutamic acid; the amino acid substitution at position 100 is alanine; the amino acid substitution at position 101 is histidine; the amino acid substitution at position 103 is serine; the amino acid substitution at position 114 is valine; the amino acid substitution at position 127 is selected from the group consisting of arginine, lysine, and histidine; the amino acid substitution at position 158 is cysteine; the amino acid substitution at position 159 is leucine; the amino acid substitution at position 160 is valine; the amino acid substitution at position 178 is valine; the amino acid substitution at position 192 is glycine; the amino acid substitution at position 196 is tryptophan; and the amino acid substitution at position 200 is histidine. In further embodiments, the present invention is directed toward modified TetR(B) proteins that comprise the single or multiple amino acid substitutions at positions of the TetR(B) protein that correspond to those identified in the revTetR(BD) chimeras of Table 1.

In another embodiment of the present invention is directed toward a modified TetR(C) protein comprising an amino acid substitution at a position selected from the group consisting of positions 59, 70, 71, 91, 95, 96, 99, 100, 101, 103, 114, 127, 158, 159, 164, 182, 196, 200, and 204 of the TetR(C) protein as depicted in SEQ ID NO: 38, wherein said modified TetR(C) protein binds a TetR(C) operator sequence with greater affinity in the presence of tetracycline than in the absence of tetracycline. In particular aspects of this embodiment: the amino acid substitution at position 59 is selected from the group consisting of glycine, serine, threonine, cysteine, tyrosine, asparagine and glutamine; the amino acid substitution at position 70 is selected from the group consisting of isoleucine, valine, phenylalanine, methionine, and tryptophan; the amino acid substitution at position 71 is selected from the group consisting of leucine, isoleucine, valine, phenylalanine, methionine, and tryptophan; the amino acid substitution at position 91 is selected from the group consisting of glycine, serine, threonine, cysteine, tyrosine, asparagine, and glutamine; the amino acid substitution at position 95 is selected from the group consisting of glycine, serine, threonine, cysteine, tyrosine, asparagine, glutamine, alanine, and glutamic acid; the amino acid substitution at position 96 is selected from the group consisting of aspartic acid, glutamic acid, arginine, lysine, and histidine; the amino acid substitution at position 99 is selected from the group consisting of aspartic acid, and glutamic acid; the amino acid substitution at position 100 is selected from the group consisting of alanine, leucine, isoleucine, valine, proline, phenylalanine, tryptophan, and methionine; the amino acid substitution at position 101 is selected from the group consisting of arginine, lysine, and histidine; the amino acid substitution at position 103 is selected from the group consisting of glycine, serine, cysteine, tyrosine, asparagine, and glutamine; the amino acid substitution at position 114 is selected from the group consisting of alanine, leucine, isoleucine, valine, proline, phenylalanine, tryptophan, and methionine; the amino acid substitution at position 127 is selected from the group consisting of arginine, lysine, and histidine; the amino acid substitution at position 158 is selected from the group consisting of glycine, serine, threonine, cysteine, tyrosine, and glutamine; the amino acid substitution at position 159 is selected from the group consisting of methionine, leucine, isoleucine, phenylalanine, and tryptophan; the amino acid substitution at position 164 is selected from the group consisting of methionine, leucine, valine, proline, phenylalanine, and tryptophan; the amino acid substitution at position 182 is selected from the group consisting of methionine, leucine, isoleucine, valine, proline, phenylalanine, and tryptophan; the amino acid substitution at position 196 is selected from the group consisting of glycine, threonine, cysteine, tyrosine, asparagine, and glutamine; the amino acid substitution at position 200 is selected from the group consisting of alanine, leucine, isoleucine, valine, proline, phenylalanine, tryptophan, and tyrosine; and the amino acid substitution at position 201 is selected from the group consisting of arginine, lysine, and histidine.

In another embodiment, the present invention is directed toward a modified TetR(C) protein comprising an amino acid substitution at a position selected from the group consisting of positions 59, 70, 71, 91, 95, 96, 99, 100, 101, 103, 114, 127, 158, 159, 164, 182, 196, 200, and 204 of the TetR(C) protein as depicted in SEQ ID NO: 38, wherein said modified TetR(C) protein binds a TetR(C) operator sequence with greater affinity in the presence of tetracycline than in the absence of tetracycline, wherein: the amino acid substitution at position 59 is asparagine; the amino acid substitution at position 70 is valine; the amino acid substitution at position 71 is valine; the amino acid substitution at position 91 is glutamine; the amino acid substitution at position 95 is selected from the group consisting of glycine and glutamic acid; the amino acid substitution at position 95 is glycine; the amino acid substitution at position 95 is glutamic acid; the amino acid substitution at position 96 is arginine; the amino acid substitution at position 96 is glutamic acid; the amino acid substitution at position 99 is glutamic acid; the amino acid substitution at position 100 is alanine; the amino acid substitution at position 101 is histidine; the amino acid substitution at position 103 is serine; the amino acid substitution at position 114 is valine; the amino acid substitution at position 127 is selected from the group consisting of arginine, lysine, and histidine; the amino acid substitution at position 158 is cysteine; the amino acid substitution at position 159 is leucine; the amino acid substitution at position 164 is valine; the amino acid substitution at position 182 is valine; the amino acid substitution at position 196 is glycine; wherein the amino acid substitution at position 200 is tryptophan; and the amino acid substitution at position 204 is histidine. In further embodiments, the present invention is directed toward modified TetR(C) proteins that comprise the single or multiple amino acid substitutions at positions of the TetR(C) protein that correspond to those identified in the revTetR(BD) chimeras of Table 1.

In still another embodiment, the present invention is directed toward a modified TetR(D) protein comprising an amino acid substitution at a position selected from the group consisting of positions 59, 70, 71, 91, 95, 96, 99, 100, 101, 103, 114, 127, 158, 159, 160, 178, 192, 196, and 200 of the TetR(D) protein as depicted in SEQ ID NO: 40, wherein said modified TetR(D) protein binds a TetR(D) operator sequence with greater affinity in the presence of tetracycline than in the absence of tetracycline. In particular aspects of this embodiment: the amino acid substitution at position 59 is selected from the group consisting of glycine, serine, threonine, cysteine, tyrosine, asparagine and glutamine; the amino acid substitution at position 70 is selected from the group consisting of isoleucine, valine, phenylalanine, methionine, and tryptophan; the amino acid substitution at position 71 is selected from the group consisting of leucine, isoleucine, valine, phenylalanine, methionine, and tryptophan; the amino acid substitution at position 91 is selected from the group consisting of glycine, serine, threonine, cysteine, tyrosine, asparagine, and glutamine; the amino acid substitution at position 95 is selected from the group consisting of glycine, serine, threonine, cysteine, tyrosine, asparagine, glutamine, alanine, and glutamic acid; the amino acid substitution at position 96 is selected from the group consisting of aspartic acid, glutamic acid, arginine, lysine, and histidine; the amino acid substitution at position 99 is selected from the group consisting of aspartic acid, and glutamic acid; the amino acid substitution at position 100 is selected from the group consisting of alanine, leucine, isoleucine, valine, pro line, phenylalanine, tryptophan, and methionine; the amino acid substitution at position 101 is selected from the group consisting of arginine, lysine, and histidine; the amino acid substitution at position 103 is selected from the group consisting of glycine, serine, cysteine, tyrosine, asparagine, and glutamine; the amino acid substitution at position 114 is selected from the group consisting of alanine, leucine, isoleucine, valine, proline, phenylalanine, tryptophan, and methionine; the amino acid substitution at position 127 is selected from the group consisting of arginine, lysine, and histidine; the amino acid substitution at position 158 is selected from the group consisting of glycine, serine, threonine, cysteine, tyrosine, and glutamine; the amino acid substitution at position 159 is selected from the group consisting of methionine, leucine, isoleucine, phenylalanine, and tryptophan; the amino acid substitution at position 160 is selected from the group consisting of methionine, leucine, valine, proline, phenylalanine, and tryptophan; the amino acid substitution at position 178 is selected from the group consisting of methionine, leucine, isoleucine, valine, proline, phenylalanine, and tryptophan; the amino acid substitution at position 192 is selected from the group consisting of glycine, threonine, cysteine, tyrosine, asparagine, and glutamine; the amino acid substitution at position 196 is selected from the group consisting of alanine, leucine, isoleucine, valine, proline, phenylalanine, tryptophan, and tyrosine; the amino acid substitution at position 200 is selected from the group consisting of arginine, lysine, and histidine.

In another embodiment, the present invention is directed toward a modified TetR(D) protein comprising an amino acid substitution at a position selected from the group consisting of positions 59, 70, 71, 91, 95, 96, 99, 100, 101, 103, 114, 127, 158, 159, 160, 178, 192, 196, and 200 of the TetR(D) protein as depicted in SEQ ID NO: 40, wherein said modified TetR(D) protein binds a TetR(D) operator sequence with greater affinity in the presence of tetracycline than in the absence of tetracycline, wherein: the amino acid substitution at position 59 is asparagine; the amino acid substitution at position 70 is valine; the amino acid substitution at position 71 is valine; the amino acid substitution at position 91 is glutamine; the amino acid substitution at position 95 is selected from the group consisting of glycine and glutamic acid; the amino acid substitution at position 95 is glycine; the amino acid substitution at position 96 is arginine; the amino acid substitution at position 96 is glutamic acid; the amino acid substitution at position 99 is glutamic acid; the amino acid substitution at position 100 is alanine; the amino acid substitution at position 101 is histidine; the amino acid substitution at position 103 is serine; the amino acid substitution at position 114 is valine; the amino acid substitution at position 127 is selected from the group consisting of arginine, lysine, and histidine; the amino acid substitution at position 158 is cysteine; the amino acid substitution at position 159 is leucine; the amino acid substitution at position 160 is valine; the amino acid substitution at position 178 is valine; the amino acid substitution at position 192 is glycine; the amino acid substitution at position 196 is tryptophan; and the amino acid substitution at position 200 is histidine. In further embodiments, the present invention is directed toward modified TetR(D) proteins that comprise the single or multiple amino acid substitutions at positions of the TetR(D) protein that correspond to those identified in the revTetR(BD) chimeras of Table 1.

In a further embodiment, the present invention is directed toward a modified TetR(E) protein comprising an amino acid substitution at a position selected from the group consisting of positions 59, 70, 71, 91, 95, 96, 99, 100, 101, 103, 114, 127, 158, 159, 160, 175, 189, 193, and 197 of the TetR(E) protein as depicted in SEQ ID NO: 42, wherein said modified TetR(E) protein binds a TetR(E) operator sequence with greater affinity in the presence of tetracycline than in the absence of tetracycline. In particular aspects of this embodiment: the amino acid substitution at position 59 is selected from the group consisting of glycine, serine, threonine, cysteine, tyrosine, asparagine and glutamine; the amino acid substitution at position 70 is selected from the group consisting of isoleucine, valine, phenylalanine, methionine, and tryptophan; the amino acid substitution at position 71 is selected from the group consisting of leucine, isoleucine, valine, phenylalanine, methionine, and tryptophan; the amino acid substitution at position 91 is selected from the group consisting of glycine, serine, threonine, cysteine, tyrosine, asparagine, and glutamine; the amino acid substitution at position 95 is selected from the group consisting of glycine, serine, threonine, cysteine, tyrosine, asparagine, glutamine, alanine, and glutamic acid; the amino acid substitution at position 96 is selected from the group consisting of aspartic acid, glutamic acid, arginine, lysine, and histidine; the amino acid substitution at position 99 is selected from the group consisting of aspartic acid, and glutamic acid; the amino acid substitution at position 100 is selected from the group consisting of alanine, leucine, isoleucine, valine, proline, phenylalanine, tryptophan, and methionine; the amino acid substitution at position 101 is selected from the group consisting of arginine, lysine, and histidine; the amino acid substitution at position 103 is selected from the group consisting of glycine, serine, cysteine, tyrosine, asparagine, and glutamine; the amino acid substitution at position 114 is selected from the group consisting of alanine, leucine, isoleucine, valine, proline, phenylalanine, tryptophan, and methionine; the amino acid substitution at position 127 is selected from the group consisting of arginine, lysine, and histidine; the amino acid substitution at position 158 is selected from the group consisting of glycine, serine, threonine, cysteine, tyrosine, and glutamine; the amino acid substitution at position 159 is selected from the group consisting of methionine, leucine, isoleucine, phenylalanine, and tryptophan; the amino acid substitution at position 160 is selected from the group consisting of methionine, leucine, valine, proline, phenylalanine, and tryptophan; the amino acid substitution at position 175 is selected from the group consisting of methionine, leucine, isoleucine, valine, proline, phenylalanine, and tryptophan; the amino acid substitution at position 193 is selected from the group consisting of glycine, threonine, cysteine, tyrosine, asparagine, and glutamine; the amino acid substitution at position 197 is selected from the group consisting of alanine, leucine, isoleucine, valine, proline, phenylalanine, tryptophan, and tyrosine; and the amino acid substitution at position 201 is selected from the group consisting of arginine, lysine, and histidine.

In a still further embodiment, the present invention is directed toward a modified TetR(E) protein comprising an amino acid substitution at a position selected from the group consisting of positions 59, 70, 71, 91, 95, 96, 99, 100, 101, 103, 114, 127, 158, 159, 160, 175, 189, 193, and 197 of the TetR(E) protein as depicted in SEQ ID NO: 42, wherein said modified TetR(E) protein binds a TetR(E) operator sequence with greater affinity in the presence of tetracycline than in the absence of tetracycline, where: the amino acid substitution at position 59 is asparagine; the amino acid substitution at position 70 is valine; the amino acid substitution at position 71 is valine; the amino acid substitution at position 91 is glutamine; the amino acid substitution at position 95 is selected from the group consisting of glycine and glutamic acid; the amino acid substitution at position 95 is glycine; the amino acid substitution at position 95 is glutamic acid; the amino acid substitution at position 96 is arginine; the amino acid substitution at position 96 is glutamic acid; the amino acid substitution at position 99 is glutamic acid; the amino acid substitution at position 100 is alanine; the amino acid substitution at position 101 is histidine; the amino acid substitution at position 103 is serine; the amino acid substitution at position 114 is valine; the amino acid substitution at position 127 is selected from the group consisting of arginine, lysine, and histidine; the amino acid substitution at position 158 is cysteine; the amino acid substitution at position 159 is leucine; the amino acid substitution at position 160 is valine; the amino acid substitution at position 179 is valine; the amino acid substitution at position 193 is glycine; the amino acid substitution at position 197 is tryptophan; and the amino acid substitution at position 201 is histidine. In further embodiments, the present invention is directed toward modified TetR(E) proteins that comprise the single or multiple amino acid substitutions at positions of the TetR(E) protein that correspond to those identified in the revTetR(BD) chimeras of Table 1.

In still another embodiment, the present invention is directed toward, a modified TetR(G) protein comprising an amino acid substitution at a position selected from the group consisting of positions 59, 70, 71, 91, 95, 96, 99, 100, 101, 103, 114, 127, 160, 161, 162, 180, 194, 198, and 202 of the TetR(G) protein as depicted in SEQ ID NO: 44, wherein said modified TetR(G) protein binds a TetR(G) operator sequence with greater affinity in the presence of tetracycline than in the absence of tetracycline. In particular aspects of this embodiment: the amino acid substitution at position 59 is selected from the group consisting of glycine, serine, threonine, cysteine, tyrosine, asparagine and glutamine; the amino acid substitution at position 70 is selected from the group consisting of isoleucine, valine, phenylalanine, methionine, and tryptophan; the amino acid substitution at position 71 is selected from the group consisting of leucine, isoleucine, valine, phenylalanine, methionine, and tryptophan; the amino acid substitution at position 91 is selected from the group consisting of glycine, serine, threonine, cysteine, tyrosine, asparagine, and glutamine; the amino acid substitution at position 95 is selected from the group consisting of glycine, serine, threonine, cysteine, tyrosine, asparagine, glutamine, alanine, and glutamic acid; the amino acid substitution at position 96 is selected from the group consisting of aspartic acid, glutamic acid, arginine, lysine, and histidine; the amino acid substitution at position 99 is selected from the group consisting of aspartic acid, and glutamic acid; the amino acid substitution at position 100 is selected from the group consisting of alanine, leucine, isoleucine, valine, proline, phenylalanine, tryptophan, and methionine; the amino acid substitution at position 101 is selected from the group consisting of arginine, lysine, and histidine; the amino acid substitution at position 103 is selected from the group consisting of glycine, serine, cysteine, tyrosine, asparagine, and glutamine; the amino acid substitution at position 114 is selected from the group consisting of alanine, leucine, isoleucine, valine, proline, phenylalanine, tryptophan, and methionine; the amino acid substitution at position 127 is selected from the group consisting of arginine, lysine, and histidine; the amino acid substitution at position 160 is selected from the group consisting of glycine, serine, threonine, cysteine, tyrosine, and glutamine; the amino acid substitution at position 161 is selected from the group consisting of methionine, leucine, isoleucine, phenylalanine, and tryptophan; the amino acid substitution at position 162 is selected from the group consisting of methionine, leucine, valine, proline, phenylalanine, and tryptophan; the amino acid substitution at position 180 is selected from the group consisting of methionine, leucine, isoleucine, valine, proline, phenylalanine, and tryptophan; the amino acid substitution at position 194 is selected from the group consisting of glycine, threonine, cysteine, tyrosine, asparagine, and glutamine; the amino acid substitution at position 198 is selected from the group consisting of alanine, leucine, isoleucine, valine, proline, phenylalanine, tryptophan, and tyrosine; and the amino acid substitution at position 202 is selected from the group consisting of arginine, lysine, and histidine.

In a still further embodiment, the present invention is directed toward a modified TetR(G) protein comprising an amino acid substitution at a position selected from the group consisting of positions 59, 70, 71, 91, 95, 96, 99, 100, 101, 103, 114, 127, 160, 161, 162, 180, 194, 198, and 202 of the TetR(G) protein as depicted in SEQ ID NO: 44, wherein said modified TetR(G) protein binds a TetR(G) operator sequence with greater affinity in the presence of tetracycline than in the absence of tetracycline and where: the amino acid substitution at position 59 is asparagine; the amino acid substitution at position 70 is valine; the amino acid substitution at position 71 is valine; the amino acid substitution at position 91 is glutamine; the amino acid substitution at position 95 is selected from the group consisting of glycine and glutamic acid; the amino acid substitution at position 95 is glycine; the amino acid substitution at position 95 is glutamic acid; the amino acid substitution at position 96 is arginine; the amino acid substitution at position 96 is glutamic acid; the amino acid substitution at position 99 is glutamic acid; the amino acid substitution at position 100 is alanine; the amino acid substitution at position 101 is histidine; the amino acid substitution at position 103 is serine; the amino acid substitution at position 114 is valine; the amino acid substitution at position 127 is selected from the group consisting of arginine, lysine, and histidine; the amino acid substitution at position 160 is cysteine; the amino acid substitution at position 161 is leucine; the amino acid substitution at position 162 is valine; the amino acid substitution at position 180 is valine; the amino acid substitution at position 194 is glycine; the amino acid substitution at position 198 is tryptophan; and the amino acid substitution at position 202 is histidine. In further embodiments, the present invention is directed toward modified TetR(G) proteins that comprise the single or multiple amino acid substitutions at positions of the TetR(G) protein that correspond to those identified in the revTetR(BD) chimeras of Table 1.

In another embodiment, the present invention is directed toward a modified TetR(H) protein comprising an amino acid substitution at a position selected from the group consisting of positions 59, 70, 71, 91, 95, 96, 99, 100, 101, 103, 114, 127, 158, 159, 160, 180, 194, 198, and 202 of the TetR(H) protein as depicted in SEQ ID NO: 46, wherein said modified TetR(H) protein binds a TetR(H) operator sequence with greater affinity in the presence of tetracycline than in the absence of tetracycline. In particular aspects of this embodiment: the amino acid substitution at position 59 is selected from the group consisting of glycine, serine, threonine, cysteine, tyrosine, asparagine and glutamine; the amino acid substitution at position 70 is selected from the group consisting of isoleucine, valine, phenylalanine, methionine, and tryptophan; the amino acid substitution at position 71 is selected from the group consisting of leucine, isoleucine, valine, phenylalanine, methionine, and tryptophan; the amino acid substitution at position 91 is selected from the group consisting of glycine, serine, threonine, cysteine, tyrosine, asparagine, and glutamine; the amino acid substitution at position 95 is selected from the group consisting of glycine, serine, threonine, cysteine, tyrosine, asparagine, glutamine, alanine, and glutamic acid; the amino acid substitution at position 96 is selected from the group consisting of aspartic acid, glutamic acid, arginine, lysine, and histidine; the amino acid substitution at position 99 is selected from the group consisting of aspartic acid, and glutamic acid; the amino acid substitution at position 100 is selected from the group consisting of alanine, leucine, isoleucine, valine, proline, phenylalanine, tryptophan, and methionine; the amino acid substitution at position 101 is selected from the group consisting of arginine, lysine, and histidine; the amino acid substitution at position 103 is selected from the group consisting of glycine, serine, cysteine, tyrosine, asparagine, and glutamine; the amino acid substitution at position 114 is selected from the group consisting of alanine, leucine, isoleucine, valine, proline, phenylalanine, tryptophan, and methionine; the amino acid substitution at position 127 is selected from the group consisting of arginine, lysine, and histidine; the amino acid substitution at position 158 is selected from the group consisting of glycine, serine, threonine, cysteine, tyrosine, and glutamine; the amino acid substitution at position 159 is selected from the group consisting of methionine, leucine, isoleucine, phenylalanine, and tryptophan; the amino acid substitution at position 160 is selected from the group consisting of methionine, leucine, valine, proline, phenylalanine, and tryptophan; the amino acid substitution at position 180 is selected from the group consisting of methionine, leucine, isoleucine, valine, proline, phenylalanine, and tryptophan; the amino acid substitution at position 194 is selected from the group consisting of glycine, threonine, cysteine, tyrosine, asparagine, and glutamine; the amino acid substitution at position 198 is selected from the group consisting of alanine, leucine, isoleucine, valine, proline, phenylalanine, tryptophan, and tyrosine; and the amino acid substitution at position 202 is selected from the group consisting of arginine, lysine, and histidine.

In still another embodiment, the present invention is directed toward a modified TetR(H) protein comprising an amino acid substitution at a position selected from the group consisting of positions 59, 70, 71, 91, 95, 96, 99, 100, 101, 103, 114, 127, 158, 159, 160, 180, 194, 198, and 202 of the TetR(H) protein as depicted in SEQ ID NO: 46, wherein said modified TetR(H) protein binds a TetR(H) operator sequence with greater affinity in the presence of tetracycline than in the absence of tetracycline, where: the amino acid substitution at position 59 is asparagine; the amino acid substitution at position 70 is valine; the amino acid substitution at position 71 is valine; the amino acid substitution at position 91 is glutamine; the amino acid substitution at position 95 is selected from the group consisting of glycine and glutamic acid; the amino acid substitution at position 95 is glycine; the amino acid substitution at position 95 is glutamic acid; the amino acid substitution at position 96 is arginine; the amino acid substitution at position 96 is glutamic acid; the amino acid substitution at position 99 is glutamic acid; the amino acid substitution at position 100 is alanine; the amino acid substitution at position 101 is histidine; the amino acid substitution at position 103 is serine; the amino acid substitution at position 114 is valine; the amino acid substitution at position 127 is selected from the group consisting of arginine, lysine, and histidine; the amino acid substitution at position 127 is arginine; the amino acid substitution at position 127 is lysine; the amino acid substitution at position 127 is arginine; the amino acid substitution at position 127 is histidine; the amino acid substitution at position 158 is cysteine; the amino acid substitution at position 159 is leucine; the amino acid substitution at position 160 is valine; the amino acid substitution at position 180 is valine; the amino acid substitution at position 194 is glycine; the amino acid substitution at position 198 is tryptophan; and the amino acid substitution at position 202 is histidine. In further embodiments, the present invention is directed toward modified TetR(H) proteins that comprise the single or multiple amino acid substitutions at positions of the TetR(H) protein that correspond to those identified in the revTetR(BD) chimeras of Table 1.

In a still further embodiment, the present invention is directed toward a modified TetR(J) protein comprising an amino acid substitution at an amino acid position selected from the group consisting of positions 59, 70, 71, 91, 95, 96, 99, 100, 101, 103, 114, 127, 158, 159, 160, 180, 194, 198, and 202 of the TetR(J) protein as depicted in SEQ ID NO: 48, wherein said modified TetR(J) protein binds a TetR(J) operator sequence with greater affinity in the presence of tetracycline than in the absence of tetracycline. In particular aspects of this embodiment: the amino acid substitution at position 59 is selected from the group consisting of glycine, serine, threonine, cysteine, tyrosine, asparagine and glutamine; the amino acid substitution at position 70 is selected from the group consisting of isoleucine, valine, phenylalanine, methionine, and tryptophan; the amino acid substitution at position 71 is selected from the group consisting of leucine, isoleucine, valine, phenylalanine, methionine, and tryptophan; the amino acid substitution at position 91 is selected from the group consisting of glycine, serine, threonine, cysteine, tyrosine, asparagine, and glutamine; the amino acid substitution at position 95 is selected from the group consisting of glycine, serine, threonine, cysteine, tyrosine, asparagine, glutamine, alanine, and glutamic acid; the amino acid substitution at position 96 is selected from the group consisting of aspartic acid, glutamic acid, arginine, lysine, and histidine; the amino acid substitution at position 99 is selected from the group consisting of aspartic acid, and glutamic acid; the amino acid substitution at position 100 is selected from the group consisting of alanine, leucine, isoleucine, valine, pro line, phenylalanine, tryptophan, and methionine; the amino acid substitution at position 101 is selected from the group consisting of arginine, lysine, and histidine; the amino acid substitution at position 103 is selected from the group consisting of glycine, serine, cysteine, tyrosine, asparagine, and glutamine; the amino acid substitution at position 114 is selected from the group consisting of alanine, leucine, isoleucine, valine, proline, phenylalanine, tryptophan, and methionine; the amino acid substitution at position 127 is selected from the group consisting of arginine, lysine, and histidine; the amino acid substitution at position 158 is selected from the group consisting of glycine, serine, threonine, cysteine, tyrosine, and glutamine; the amino acid substitution at position 159 is selected from the group consisting of methionine, leucine, isoleucine, phenylalanine, and tryptophan; the amino acid substitution at position 160 is selected from the group consisting of methionine, leucine, valine, proline, phenylalanine, and tryptophan; the amino acid substitution at position 180 is selected from the group consisting of methionine, leucine, isoleucine, valine, proline, phenylalanine, and tryptophan; the amino acid substitution at position 194 is selected from the group consisting of glycine, threonine, cysteine, tyrosine, asparagine, and glutamine; the amino acid substitution at position 198 is selected from the group consisting of alanine, leucine, isoleucine, valine, proline, phenylalanine, tryptophan, and tyrosine; and the amino acid substitution at position 202 is selected from the group consisting of arginine, lysine, and histidine.

In another embodiment, the present invention is directed toward a modified TetR(J) protein comprising an amino acid substitution at an amino acid position selected from the group consisting of positions 59, 70, 71, 91, 95, 96, 99, 100, 101, 103, 114, 127, 158, 159, 160, 180, 194, 198, and 202 of the TetR(J) protein as depicted in SEQ ID NO: 48, wherein said modified TetR(J) protein binds a TetR(J) operator sequence with greater affinity in the presence of tetracycline than in the absence of tetracycline, where: the amino acid substitution at position 59 is asparagine; the amino acid substitution at position 70 is valine; the amino acid substitution at position 71 is valine; the amino acid substitution at position 91 is glutamine; the amino acid substitution at position 95 is selected from the group consisting of glycine and glutamic acid; the amino acid substitution at position 95 is glycine; the amino acid substitution at position 95 is glutamic acid; the amino acid substitution at position 96 is arginine; the amino acid substitution at position 96 is glutamic acid; the amino acid substitution at position 99 is glutamic acid; the amino acid substitution at position 100 is alanine; the amino acid substitution at position 101 is histidine; the amino acid substitution at position 103 is serine; the amino acid substitution at position 114 is valine; the amino acid substitution at position 127 is selected from the group consisting of arginine, lysine, and histidine; the amino acid substitution at position 158 is cysteine; the amino acid substitution at position 159 is leucine; the amino acid substitution at position 160 is valine; the amino acid substitution at position 180 is valine; the amino acid substitution at position 194 is glycine; the amino acid substitution at position 198 is tryptophan; and the amino acid substitution at position 202 is histidine. In further embodiments, the present invention is directed toward modified TetR(J) proteins that comprise the single or multiple amino acid substitutions at positions of the TetR(J) protein that correspond to those identified in the revTetR(BD) chimeras of Table 1.

In still another embodiment, the present invention is directed toward a modified TetR(Z) protein comprising an amino acid substitution at an amino acid position selected from the group consisting of positions 63, 74, 75, 95, 99, 100, 103, 104, 105, 107, 118, 137, 165, 166, 167, 181, 185, and 189 of the TetR(Z) protein as depicted in SEQ ID NO: 50, wherein said modified TetR(Z) protein binds a TetR(Z) operator sequence with greater affinity in the presence of tetracycline than in the absence of tetracycline. In particular aspects of this embodiment: the amino acid substitution at position 63 is selected from the group consisting of glycine, serine, threonine, cysteine, tyrosine, asparagine and glutamine; the amino acid substitution at position 74 is selected from the group consisting of isoleucine, valine, phenylalanine, methionine, and tryptophan; the amino acid substitution at position 75 is selected from the group consisting of leucine, isoleucine, valine, phenylalanine, methionine, and tryptophan; the amino acid substitution at position 95 is selected from the group consisting of glycine, serine, threonine, cysteine, tyrosine, asparagine, and glutamine; the amino acid substitution at position 99 is selected from the group consisting of glycine, serine, threonine, cysteine, tyrosine, asparagine, glutamine, alanine, and glutamic acid; the amino acid substitution at position 100 is selected from the group consisting of aspartic acid, glutamic acid, arginine, lysine, and histidine; the amino acid substitution at position 103 is selected from the group consisting of aspartic acid, and glutamic acid; the amino acid substitution at position 104 is selected from the group consisting of alanine, leucine, isoleucine, valine, proline, phenylalanine, tryptophan, and methionine; the amino acid substitution at position 105 is selected from the group consisting of arginine, lysine, and histidine; the amino acid substitution at position 107 is selected from the group consisting of glycine, serine, cysteine, tyrosine, asparagine, and glutamine; the amino acid substitution at position 118 is selected from the group consisting of alanine, leucine, isoleucine, valine, proline, phenylalanine, tryptophan, and methionine; the amino acid substitution at position 137 is selected from the group consisting of arginine, lysine, and histidine; the amino acid substitution at position 165 is selected from the group consisting of glycine, serine, threonine, cysteine, tyrosine, and glutamine; the amino acid substitution at position 166 is selected from the group consisting of methionine, leucine, isoleucine, phenylalanine, and tryptophan; the amino acid substitution at position 167 is selected from the group consisting of methionine, leucine, valine, proline, phenylalanine, and tryptophan; the amino acid substitution at position 181 is selected from the group consisting of glycine, threonine, cysteine, tyrosine, asparagine, and glutamine; the amino acid substitution at position 185 is selected from the group consisting of alanine, leucine, isoleucine, valine, proline, phenylalanine, tryptophan, and tyrosine; and the amino acid substitution at position 189 is selected from the group consisting of arginine, lysine, and histidine.

In a further embodiment, the present invention is directed toward a modified TetR(Z) protein comprising an amino acid substitution at an amino acid position selected from the group consisting of positions 63, 74, 75, 95, 99, 100, 103, 104, 105, 107, 118, 137, 165, 166, 167, 181, 185, and 189 of the TetR(Z) protein as depicted in SEQ ID NO: 50, wherein said modified TetR(Z) protein binds a TetR(Z) operator sequence with greater affinity in the presence of tetracycline than in the absence of tetracycline, where: the amino acid substitution at position 63 is asparagine, the amino acid substitution at position 74 is valine; the amino acid substitution at position 75 is valine; the amino acid substitution at position 95 is glutamine; the amino acid substitution at position 99 is selected from the group consisting of glycine and glutamic acid; the amino acid substitution at position 99 is glycine; the amino acid substitution at position 99 is glutamic acid; the amino acid substitution at position 100 is arginine; the amino acid substitution at position 100 is glutamic acid; the amino acid substitution at position 103 is glutamic acid; the amino acid substitution at position 104 is alanine; the amino acid substitution at position 105 is histidine; the amino acid substitution at position 107 is serine; the amino acid substitution at position 118 is valine; the amino acid substitution at position 137 is selected from the group consisting of arginine, lysine, and histidine; the amino acid substitution at position 165 is cysteine; the amino acid substitution at position 166 is leucine; the amino acid substitution at position 167 is valine; the amino acid substitution at position 181 is glycine; the amino acid substitution at position 185 is tryptophan; and the amino acid substitution at position 189 is histidine. In further embodiments, the present invention is directed toward modified TetR(Z) proteins that comprise the single or multiple amino acid substitutions at positions of the TetR(Z) protein that correspond to those identified in the revTetR(BD) chimeras of Table 1.

In still further embodiment, the present invention is directed toward a modified TetR(A) protein comprising a plurality of amino acid substitutions at positions selected from the group consisting of positions 59, 70, 71, 91, 95, 96, 98, 99, 100, 101, 103, 110, 114, 127, 157, 158, 159, 160, 179, 189, 193, 195, 197, 201, and 206 of the TetR(A) protein as depicted in SEQ ID NO: 34, wherein said TetR(A) protein binds a TetR(A) operator sequence with greater affinity in the presence of tetracycline than in the absence of tetracycline. In particular aspects of this embodiment: the amino acid substitution at position 98 is histidine; the amino acid substitution at position 110 is selected from the group consisting of alanine, leucine, valine, proline, phenylalanine and tryptophan; the amino acid substitution at position 157 is selected from the group consisting of glycine, serine, threonine, cysteine, tyrosine, asparagine, and glutamine; the amino acid substitution at position 189 is selected from the group consisting of glycine, serine, threonine, cysteine, tyrosine, and asparagine; the amino acid substitution at position 195 is selected from the group consisting of alanine, leucine, proline, phenylalanine, and tryptophan; the amino acid substitution at position 206 is selected from the group consisting of glycine, serine, threonine, cysteine, tyrosine, asparagine, and glutamine; the amino acid substitutions are at positions 96 and 159 and the amino acid substitution at position 96 is arginine and the amino acid substitution at position 159 is leucine; the amino acid substitutions are at positions 96 and 159 and the amino acid substitution at position 96 is glutamic acid and the amino acid substitution at position 159 is leucine; the amino acid substitutions are at positions 96 and 110 and the amino acid substitution at position 96 is arginine and the amino acid substitution at position 110 is phenylalanine; the amino acid substitutions are at positions 96 and 110 and the amino acid substitution at position 96 is glutamic acid and the amino acid substitution at position 110 is phenylalanine; the amino acid substitutions are at positions 96 and 206 and the amino acid substitution at position 96 is arginine and the amino acid substitution at position 206 is serine; the amino acid substitutions are at positions 96 and 206 and the amino acid substitution at position 96 is glutamic acid and the amino acid substitution at position 110 is serine; the amino acid substitutions are at positions 99 and 158 and the amino acid substitution at position 99 is glutamic acid and the amino acid substitution at position 158 is cysteine; the amino acid substitutions are at positions 96, 103, and 114 and the amino acid substitution at position 96 is arginine, the amino acid substitution at position 103 is serine, and the amino acid substitution at position 114 is valine; the amino acid substitutions are at positions 96, 103, and 114 and the amino acid substitution at position 96 is glutamic acid, the amino acid substitution at position 103 is serine, and the amino acid substitution at position 114 is valine; the amino acid substitutions are at positions 96, 157, and 201 and the amino acid substitution at position 96 is arginine, the amino acid substitution at position 157 is asparagine, and the amino acid substitution at position 201 is histidine; the amino acid substitutions are at positions 96, 157, and 201 and the amino acid substitution at position 96 is glutamic acid, the amino acid substitution at position 157 is serine, and the amino acid substitution at position 201 is histidine; the amino acid substitutions are at positions 59, 95, and 100, and the amino acid substitution at position 59 is asparagine, the amino acid substitution at position 95 is glutamic acid, and the amino acid substitution at position 100 is alanine; the amino acid substitutions are at positions 59, 95, and 100, and the amino acid substitution at position 59 is asparagine, the amino acid substitution at position 95 is glycine, and the amino acid substitution at position 100 is alanine; the amino acid substitutions are at positions 160, 179, and 197, and the amino acid substitution at position 160 is valine, the amino acid substitution at position 179 is valine, and the amino acid substitution at position 197 is tryptophan; the amino acid substitutions are at positions 70, 91, and 99, and the amino acid substitution at position 70 is valine, the amino acid substitution at position 91 is glutamine, and the amino acid substitution at position 99 is glutamic acid; the amino acid substitutions are at positions 71, 95, and 127, and the amino acid substitution at position 71 is valine, the amino acid substitution at position 95 is glutamic acid, and the amino acid substitution at position 127 arginine; the amino acid substitutions are at positions 71, 95, and 127, and the amino acid substitution at position 71 is valine, the amino acid substitution at position 95 is arginine, and the amino acid substitution at position 127 arginine; and the amino acid substitutions are at positions 59, 101, and 192, and the amino acid substitution at position 59 is asparagine, the amino acid substitution at position 101 is histidine, and the amino acid substitution at position 193 glycine.

In another embodiment, the present invention is directed toward a modified TetR(B) protein comprising a plurality of amino acid substitutions at positions selected from the group consisting of positions 59, 70, 71, 91, 95, 96, 98, 99, 100, 101, 103, 110, 114, 127, 157, 158, 159, 160, 178, 188, 192, 194, 196, 200, and 205 of the TetR(B) protein as depicted in SEQ ID NO: 36, wherein said modified TetR(B) protein binds a TetR(B) operator sequence with greater affinity in the presence of tetracycline than in the absence of tetracycline. In particular aspects of this embodiment: the amino acid substitution at position 98 is arginine; the amino acid substitution at position 98 is histidine; the amino acid substitution at position 110 is selected from the group consisting of alanine, leucine, valine, proline, phenylalanine and tryptophan; the amino acid substitution at position 157 is selected from the group consisting of glycine, serine, threonine, cysteine, tyrosine, asparagine, and glutamine; the amino acid substitution at position 188 is selected from the group consisting of glycine, serine, threonine, cysteine, tyrosine, glutamine, and asparagine; the amino acid substitution at position 194 is selected from the group consisting of alanine, leucine, valine, proline, phenylalanine, and tryptophan; the amino acid substitution at position 205 is selected from the group consisting of glycine, serine, threonine, cysteine, tyrosine, asparagine, and glutamine; the amino acid substitutions are at positions 96 and 159 and the amino acid substitution at position 96 is arginine and the amino acid substitution at position 159 is leucine; the amino acid substitutions are at positions 96 and 159 and the amino acid substitution at position 96 is glutamic acid and the amino acid substitution at position 159 is leucine; the amino acid substitutions are at positions 96 and 188, and the amino acid substitution at position 96 is arginine and the amino acid substitution at position 188 is glutamine; the amino acid substitutions are at positions 96 and 188, and the amino acid substitution at position 96 glutamic acid, and the amino acid substitution at position 188 is glutamine; the amino acid substitutions are at positions 96 and 110, and the amino acid substitution at position 96 is arginine and the amino acid substitution at position 110 is phenylalanine; the amino acid substitutions are at positions 96 and 110, and the amino acid substitution at position 96 is glutamic acid and the amino acid substitution at position 110 is phenylalanine; the amino acid substitutions are at positions 99 and 194, and the amino acid substitution at position 99 is glutamic acid and the amino acid substitution at position 194 is valine; the amino acid substitutions are at positions 99 and 158, and the amino acid substitution at position 99 is glutamic acid and the amino acid substitution at position 158 is cysteine; the amino acid substitutions are at positions 96, 103, and 114, and the amino acid substitution at position 96 is arginine, the amino acid substitution at position 103 is serine, and the amino acid substitution at position 114 is valine; the amino acid substitutions are at positions 96, 103, and 114, and the amino acid substitution at position 96 is glutamic acid, the amino acid substitution at position 103 is serine, and the amino acid substitution at position 114 is valine; the amino acid substitutions are at positions 96, 157, and 200, and the amino acid substitution at position 96 is arginine, the amino acid substitution at position 157 is asparagine, and the amino acid substitution at position 200 is histidine; the amino acid substitutions are at positions 96, 157, and 200, and the amino acid substitution at position 96 is glutamic acid, the amino acid substitution at position 157 is serine, and the amino acid substitution at position 200 is histidine; the amino acid substitutions are at positions 59, 95, and 100, and the amino acid substitution at position 59 is asparagine, the amino acid substitution at position 95 is glutamic acid, and the amino acid substitution at position 100 is alanine; the amino acid substitutions are at positions 59, 95, and 100, and the amino acid substitution at position 59 is asparagine, the amino acid substitution at position 95 is glycine, and the amino acid substitution at position 100 is alanine; the amino acid substitutions are at positions 160, 178, and 196, and the amino acid substitution at position 160 is valine, the amino acid substitution at position 178 is valine, and the amino acid substitution at position 196 is tryptophan, and the amino acid substitutions are at positions 70, 91, and 99; the amino acid substitution at position 70 is valine, the amino acid substitution at position 91 is glutamine, and the amino acid substitution at position 99 is glutamic acid; the amino acid substitutions are at positions 71, 95, and 127, and the amino acid substitution at position 71 is valine, the amino acid substitution at position 95 is glutamic acid, and the amino acid substitution at position 127 arginine; the amino acid substitutions are at positions 71, 95, and 127, and the amino acid substitution at position 71 is valine, the amino acid substitution at position 95 is arginine, and the amino acid substitution at position 127 arginine; the amino acid substitutions are at positions 59, 98, 101, and 192, and the amino acid substitution at position 59 is asparagine, the amino acid substitution at position 98 is arginine, the amino acid substitution at position 101 is histidine, and the amino acid substitution at position 193 is glycine.

In a further embodiment, the present invention is directed toward a modified TetR(C) protein comprising a plurality of amino acid substitutions at positions selected from the group consisting of positions 59, 70, 71, 91, 95, 96, 98, 99, 100, 101, 103, 110, 114, 127, 157, 158, 159, 164, 182, 192, 196, 198, 200, 204, and 209 of the TetR(C) protein as depicted in SEQ ID NO: 35, wherein said TetR(C) protein binds a TetR(C) operator sequence with greater affinity in the presence of tetracycline than in the absence of tetracycline. In particular aspects of this embodiment, the amino acid substitution at position 98 is histidine; the amino acid substitution at position 110 is selected from the group consisting of alanine, leucine, valine, proline, phenylalanine and tryptophan; the amino acid substitution at position 157 is selected from the group consisting of glycine, serine, threonine, cysteine, tyrosine, asparagine, and glutamine; the amino acid substitution at position 192 is selected from the group consisting of glycine, serine, threonine, cysteine, tyrosine, glutamine, and asparagine; the amino acid substitution at position 198 is selected from the group consisting of alanine, leucine, valine, proline, phenylalanine, and tryptophan; the amino acid substitution at position 209 is selected from the group consisting of glycine, serine, threonine, cysteine, tyrosine, asparagine, and glutamine; the amino acid substitutions are at positions 96 and 159, and the amino acid substitution at position 96 is arginine and the amino acid substitution at position 159 is leucine; the amino acid substitutions are at positions 96 and 159, and the amino acid substitution at position 96 is glutamic acid and the amino acid substitution at position 159 is leucine; the amino acid substitutions are at positions 96 and 192, and the amino acid substitution at position 96 is arginine and the amino acid substitution at position 192 is glutamine; the amino acid substitutions are at positions 96 and 192, and the amino acid substitution at position 96 is glutamic acid, and the amino acid substitution at position 192 is glutamine; the amino acid substitutions are at positions 96 and 110, and the amino acid substitution at position 96 is arginine and the amino acid substitution at position 110 is phenylalanine; the amino acid substitutions are at positions 96 and 110, and the amino acid substitution at position 96 is glutamic acid and the amino acid substitution at position 110 is phenylalanine; the amino acid substitutions are at positions 96 and 209, and the amino acid substitution at position 96 is arginine and the amino acid substitution at position 209 is serine; the amino acid substitutions are at positions 96 and 209, and the amino acid substitution at position 96 is glutamic acid and the amino acid substitution at position 209 is serine; the amino acid substitutions are at positions 99 and 198, and the amino acid substitution at position 198 is valine; the amino acid substitutions are at positions 99 and 158, and the amino acid substitution at position 99 is glutamic acid and the amino acid substitution at position 158 is cysteine; the amino acid substitutions are at positions 96, 103, and 114, and the amino acid substitution at position 96 is arginine, the amino acid substitution at position 103 is serine, and the amino acid substitution at position 114 is valine; the amino acid substitutions are at positions 96, 103, and 114, and the amino acid substitution at position 96 is glutamic acid, the amino acid substitution at position 103 is serine, and the amino acid substitution at position 114 is valine; the amino acid substitutions are at positions 96, 157, and 204, and the amino acid substitution at position 96 is arginine, the amino acid substitution at position 157 is asparagine, and the amino acid substitution at position 204 is histidine; the amino acid substitutions are at positions 96, 157, and 204, and the amino acid substitution at position 96 is glutamic acid, the amino acid substitution at position 157 is serine, and the amino acid substitution at position 204 is histidine; the amino acid substitutions are at positions 59, 95, and 100, and the amino acid substitution at position 59 is asparagine, the amino acid substitution at position 95 is glutamic acid, and the amino acid substitution at position 100 is alanine; the amino acid substitutions are at positions 59, 95, and 100, and the amino acid substitution at position 59 is asparagine, the amino acid substitution at position 95 is glycine, and the amino acid substitution at position 100 is alanine; the amino acid substitutions are at positions 164, 182, and 200, and the amino acid substitution at position 164 is valine, the amino acid substitution at position 182 is valine, and the amino acid substitution at position 200 is tryptophan; the amino acid substitutions are at positions 70, 91, and 99, and the amino acid substitution at position 70 is valine, the amino acid substitution at position 91 is glutamine, and the amino acid substitution at position 99 is glutamic acid; the amino acid substitutions are at positions 71, 95, and 127, and the amino acid substitution at position 71 is valine, the amino acid substitution at position 95 is glutamic acid, and the amino acid substitution at position 127 arginine; the amino acid substitutions are at positions 71, 95, and 127, and the amino acid substitution at position 71 is valine, the amino acid substitution at position 95 is arginine, and the amino acid substitution at position 127 arginine, the amino acid substitutions are at positions 59, 101, and 196, and the amino acid substitution at position 59 is asparagine, the amino acid substitution at position 101 is histidine, and the amino acid substitution at position 196 glycine.

In a still further embodiment, the present invention is directed toward a modified TetR(D) protein comprising a plurality of amino acid substitutions at positions selected from the group consisting of positions 59, 70, 71, 91, 95, 96, 98, 99, 100, 101, 103, 110, 114, 127, 157, 158, 159, 160, 178, 188, 192, 194, 196, 200, and 205 of the TetR(D) protein as depicted in SEQ ID NO: 36, wherein said TetR(D) protein binds a TetR(D) operator sequence with greater affinity in the presence of tetracycline than in the absence of tetracycline. In particular aspects of this embodiment, the amino acid substitution at position 98 is arginine; the amino acid substitution at position 110 is selected from the group consisting of alanine, leucine, valine, proline, phenylalanine and tryptophan, the amino acid substitution at position 157 is selected from the group consisting of glycine, serine, threonine, cysteine, tyrosine, asparagine, and glutamine; the amino acid substitution at position 188 is selected from the group consisting of glycine, serine, threonine, cysteine, tyrosine, glutamine, and asparagine; the amino acid substitution at position 194 is selected from the group consisting of alanine, leucine, valine, proline, phenylalanine, and tryptophan; the amino acid substitution at position 205 is selected from the group consisting of glycine, serine, threonine, cysteine, tyrosine, asparagine, and glutamine; the amino acid substitutions are at positions 96 and 159, and the amino acid substitution at position 96 is arginine and the amino acid substitution at position 159 is leucine; the amino acid substitutions are at positions 96 and 159, and the amino acid substitution at position 96 is glutamic acid and the amino acid substitution at position 159 is leucine; the amino acid substitutions are at positions 96 and 188, and the amino acid substitution at position 96 is arginine and the amino acid substitution at position 188 is glutamine; the amino acid substitutions are at positions 96 and 188, and the amino acid substitution at position 96 is glutamic acid, and the amino acid substitution at position 188 is glutamine; the amino acid substitutions are at positions 96 and 110, and the amino acid substitution at position 96 is arginine and the amino acid substitution at position 110 is phenylalanine; the amino acid substitutions are at positions 96 and 110, and the amino acid substitution at position 96 is glutamic acid and the amino acid substitution at position 110 is phenylalanine; the amino acid substitutions are at positions 96 and 205, and the amino acid substitution at position 96 is arginine and the amino acid substitution at position 205 is serine; the amino acid substitutions are at positions 96 and 205, and the amino acid substitution at position 96 is glutamic acid and the amino acid substitution at position 205 is serine; the amino acid substitutions are at positions 99 and 194, and the amino acid substitution at position 99 is glutamic acid and the amino acid substitution at position 194 is valine; the amino acid substitutions are at positions 99 and 158, and the amino acid substitution at position 99 is glutamic acid and the amino acid substitution at position 158 is cysteine; the amino acid substitutions are at positions 96, 103, and 114, and the amino acid substitution at position 96 is arginine, the amino acid substitution at position 103 is serine, and the amino acid substitution at position 114 is valine; the amino acid substitutions are at positions 96, 103, and 114, and the amino acid substitution at position 96 is glutamic acid, the amino acid substitution at position 103 is serine, and the amino acid substitution at position 114 is valine; the amino acid substitutions are at positions 96, 157, and 200, and the amino acid substitution at position 96 is arginine, the amino acid substitution at position 157 is asparagine, and the amino acid substitution at position 200 is histidine; the amino acid substitutions are at positions 96, 157, and 200, and the amino acid substitution at position 96 is glutamic acid, the amino acid substitution at position 157 is serine, and the amino acid substitution at position 200 is histidine; the amino acid substitutions are at positions 59, 95, and 100, and the amino acid substitution at position 59 is asparagine, the amino acid substitution at position 95 is glutamic acid, and the amino acid substitution at position 100 is alanine; the amino acid substitutions are at positions 59, 95, and 100, and the amino acid substitution at position 59 is asparagine, the amino acid substitution at position 95 is glycine, and the amino acid substitution at position 100 is alanine; the amino acid substitutions are at positions 160, 178, and 196, and the amino acid substitution at position 160 is valine, the amino acid substitution at position 178 is valine, and the amino acid substitution at position 196 is tryptophan; the amino acid substitutions are at positions 70, 91, and 99, and the amino acid substitution at position 70 is valine, the amino acid substitution at position 91 is glutamine, and the amino acid substitution at position 99 is glutamic acid; the amino acid substitutions are at positions 71, 95, and 127, and the amino acid substitution at position 71 is valine, the amino acid substitution at position 95 is glutamic acid, and the amino acid substitution at position 127 arginine; the amino acid substitutions are at positions 71, 95, and 127, and the amino acid substitution at position 71 is valine, the amino acid substitution at position 95 is arginine, and the amino acid substitution at position 127 arginine; the amino acid substitutions are at positions 59, 98, 101, and 196, and the amino acid substitution at position 59 is asparagine, the amino acid substitution at position 98 is arginine, the amino acid substitution at position 101 is histidine, and the amino acid substitution at position 196 glycine.

In another embodiment, the present invention is directed toward a modified TetR(E) protein comprising a plurality of amino acid substitutions at positions selected from the group consisting of positions 59, 70, 71, 91, 95, 96, 98, 99, 100, 101, 103, 110, 114, 127, 157, 158, 159, 160, 175, 185, 189, 191, 193, 197, and 202 of the TetR(E) protein as depicted in SEQ ID NO: 37, wherein said modified TetR(E) protein binds a TetR(E) operator sequence with greater affinity in the presence of tetracycline than in the absence of tetracycline. In particular aspects of this embodiment: the amino acid substitution at position 98 is histidine; the amino acid substitution at position 110 is selected from the group consisting of alanine, leucine, valine, proline, phenylalanine and tryptophan; the amino acid substitution at position 157 is selected from the group consisting of glycine, serine, threonine, cysteine, tyrosine, asparagine, and glutamine; the amino acid substitution at position 185 is selected from the group consisting of glycine, serine, threonine, cysteine, tyrosine, glutamine, and asparagine; the amino acid substitution at position 191 is selected from the group consisting of alanine, leucine, valine, proline, phenylalanine, and tryptophan; the amino acid substitution at position 202 is selected from the group consisting of glycine, serine, threonine, cysteine, tyrosine, asparagine, and glutamine; the amino acid substitutions are at positions 96 and 159, and the amino acid substitution at position 96 is arginine and the amino acid substitution at position 159 is leucine; the amino acid substitutions are at positions 96 and 159, and the amino acid substitution at position 96 is glutamic acid and the amino acid substitution at position 159 is leucine; the amino acid substitutions are at positions 96 and 185, and the amino acid substitution at position 96 is arginine and the amino acid substitution at position 185 is glutamine; the amino acid substitutions are at positions 96 and 185, and the amino acid substitution at position 96 is glutamic acid, and the amino acid substitution at position 185 is glutamine; the amino acid substitutions are at positions 96 and 202, and the amino acid substitution at position 96 is arginine and the amino acid substitution at position 202 is serine; the amino acid substitutions are at positions 96 and 202, and the amino acid substitution at position 96 is glutamic acid and the amino acid substitution at position 202 is serine; the amino acid substitutions are at positions 99 and 191, and the amino acid substitution at position 99 is glutamic acid and the amino acid substitution at position 191 is valine; the amino acid substitutions are at positions 99 and 158, and the amino acid substitution at position 99 is glutamic acid and the amino acid substitution at position 158 is cysteine; the amino acid substitutions are at positions 96, 103, and 114, and the amino acid substitution at position 96 is arginine, the amino acid substitution at position 103 is serine, and the amino acid substitution at position 114 is valine; the amino acid substitutions are at positions 96, 103, and 114, and the amino acid substitution at position 96 is glutamic acid, the amino acid substitution at position 103 is serine, and the amino acid substitution at position 114 is valine; the amino acid substitutions are at positions 96, 157, and 197, and the amino acid substitution at position 96 is arginine, the amino acid substitution at position 157 is asparagine, and the amino acid substitution at position 197 is histidine; the amino acid substitutions are at positions 96, 157, and 197, and the amino acid substitution at position 96 is glutamic acid, the amino acid substitution at position 157 is serine, and the amino acid substitution at position 197 is histidine; the amino acid substitutions are at positions 59, 95, and 100, and the amino acid substitution at position 59 is asparagine, the amino acid substitution at position 95 is glutamic acid, and the amino acid substitution at position 100 is alanine; the amino acid substitutions are at positions 59, 95, and 100, and the amino acid substitution at position 59 is asparagine, the amino acid substitution at position 95 is glycine, and the amino acid substitution at position 100 is alanine; the amino acid substitutions are at positions 160, 175, and 193, and the amino acid substitution at position 160 is valine, the amino acid substitution at position 175 is valine, and the amino acid substitution at position 193 is tryptophan; the amino acid substitutions are at positions 70, 91, and 99, and the amino acid substitution at position 70 is valine, the amino acid substitution at position 91 is glutamine, and the amino acid substitution at position 99 is glutamic acid; the amino acid substitutions are at positions 71, 95, and 127, and the amino acid substitution at position 71 is valine, the amino acid substitution at position 95 is glutamic acid, and the amino acid substitution at position 127 arginine; the amino acid substitutions are at positions 71, 95, and 127, and the amino acid substitution at position 71 is valine, the amino acid substitution at position 95 is arginine, and the amino acid substitution at position 127 arginine; the amino acid substitutions are at positions 59, 101, and 189, and the amino acid substitution at position 59 is asparagine, the amino acid substitution at position 101 is histidine, and the amino acid substitution at position 189 glycine.

In a still further embodiment, the present invention is directed to a modified TetR(G) protein comprising a plurality of amino acid substitutions at positions selected from the group consisting of positions 59, 70, 71, 91, 95, 96, 98, 99, 100, 101, 103, 110, 114, 127, 159, 160, 161, 162, 180, 190, 194, 196, 198, 202, and 207 of the TetR(G) protein as depicted in SEQ ID NO: 38, wherein said modified TetR(G) protein binds a TetR(G) operator sequence with greater affinity in the presence of tetracycline than in the absence of tetracycline. In particular aspects of this embodiment: the amino acid substitution at position 98 is histidine; the amino acid substitution at position 110 is selected from the group consisting of alanine, leucine, valine, proline, and tryptophan; the amino acid substitution at position 159 is selected from the group consisting of glycine, serine, threonine, cysteine, tyrosine, asparagine, and glutamine; the amino acid substitution at position 190 is selected from the group consisting of glycine, serine, threonine, cysteine, tyrosine, glutamine, and asparagine; the amino: acid substitution at position 196 is selected from the group consisting of alanine, leucine, valine, proline, phenylalanine, and tryptophan; the amino acid substitution at position 207 is selected from the group consisting of glycine, serine, threonine, cysteine, tyrosine, asparagine, and glutamine; the amino acid substitutions are at positions 96 and 161, and the amino acid substitution at position 96 is arginine and the amino acid substitution at position 161 is leucine; the amino acid substitutions are at positions 96 and 161, and the amino acid substitution at position 96 is glutamic acid and the amino acid substitution at position 161 is leucine; the amino acid substitutions are at positions 96 and 190, and the amino acid substitution at position 96 is arginine and the amino acid substitution at position 190 is glutamine; the amino acid substitutions are at positions 96 and 190, and the amino acid substitution at position 96 is glutamic acid, and the amino acid substitution at position 190 is glutamine; the amino acid substitutions are at positions 96 and 207, and the amino acid substitution at position 96 is arginine and the amino acid substitution at position 207 is serine; the amino acid substitutions are at positions 96 and 207, and the amino acid substitution at position 96 is glutamic acid and the amino acid substitution at position 207 is serine; the amino acid substitutions are at positions 99 and 196, and the amino acid substitution at position 99 is glutamic acid and the amino acid substitution at position 196 is valine; the amino acid substitutions are at positions 99 and 160, and the amino acid substitution at position 99 is glutamic acid and the amino acid substitution at position 160 is cysteine; the amino acid substitutions are at positions 96, 103, and 114, and the amino acid substitution at position 96 is arginine, the amino acid substitution at position 103 is serine, and the amino acid substitution at position 114 is valine; the amino acid substitutions are at positions 96, 103, and 114, and the amino acid substitution at position 96 is glutamic acid, the amino acid substitution at position 103 is serine, and the amino acid substitution at position 114 is valine; the amino acid substitutions are at positions 96, and 202, and the amino acid substitution at position 96 is arginine, the amino acid substitution at position 202 is histidine; the amino acid substitutions are at positions 96, and 202, and the amino acid substitution at position 96 is glutamic acid, the amino acid substitution at position 202 is histidine; the amino acid substitutions are at positions 59, 95, and 100, and the amino acid substitution at position 59 is asparagine, the amino acid substitution at position 95 is glutamic acid, and the amino acid substitution at position 100 is alanine; the amino acid substitutions are at positions 59, 95, and 100, and the amino acid substitution at position 59 is asparagine, the amino acid substitution at position 95 is glycine, and the amino acid substitution at position 100 is alanine; the amino acid substitutions are at positions 162, 180, and 198, and the amino acid substitution at position 162 is valine, the amino acid substitution at position 180 is valine, and the amino acid substitution at position 198 is tryptophan; the amino acid substitutions are at positions 70, 91, and 99, and the amino acid substitution at position 70 is valine, the amino acid substitution at position 91 is glutamine, and the amino acid substitution at position 99 is glutamic acid; the amino acid substitutions are at positions 71, 95, and 127, and the amino acid substitution at position 71 is valine, the amino acid substitution at position 95 is glutamic acid, and the amino acid substitution at position 127 arginine; the amino acid substitutions are at positions 71, 95, and 127, and the amino acid substitution at position 71 is valine, the amino acid substitution at position 95 is arginine, and the amino acid substitution at position 127 arginine; the amino acid substitutions are at positions 59, 101, and 194, and the amino acid substitution at position 59 is asparagine, the amino acid substitution at position 101 is histidine, and the amino acid substitution at position 194 glycine.

In another embodiment, the present invention is directed toward a modified TetR(H) protein comprising a plurality of amino acid substitutions at positions selected from the group consisting of positions 59, 70, 71, 91, 95, 96, 98, 99, 100, 101, 103, 110, 114, 127, 157, 158, 159, 160, 180, 190, 194, 196, 198, 202, and 207 of the TetR(H) protein as depicted in SEQ ID NO: 39, wherein said modified TetR(H) protein binds a TetR(H) operator sequence with greater affinity in the presence of tetracycline than in the absence of tetracycline. In particular aspects of this embodiment, the amino acid substitution at position 98 is arginine; the amino acid substitution at position 110 is selected from the group consisting of alanine, leucine, valine, proline, phenylalanine and tryptophan; the amino acid substitution at position 157 is selected from the group consisting of glycine, serine, threonine, cysteine, tyrosine, asparagine, and glutamine; the amino acid substitution at position 190 is selected from the group consisting of glycine, serine, threonine, cysteine, tyrosine, glutamine, and asparagine; the amino acid substitution at position 196 is selected from the group consisting of alanine, leucine, valine, proline, phenylalanine, and tryptophan; the amino acid substitution at position 207 is selected from the group consisting of glycine, serine, threonine, cysteine, tyrosine, asparagine, and glutamine; the amino acid substitutions are at positions 96 and 159, and the amino acid substitution at position 96 is arginine and the amino acid substitution at position 159 is leucine; the amino acid substitutions are at positions 96 and 159, and the amino acid substitution at position 96 is glutamic acid and the amino acid substitution at position 159 is leucine; the amino acid substitutions are at positions 96 and 190, and the amino acid substitution at position 96 is arginine and the amino acid substitution at position 190 is glutamine; the amino acid substitution at position 96 is glutamic acid, and the amino acid substitution at position 188 is glutamine; the amino acid substitutions are at positions 96 and 207, and the amino acid substitution at position 96 is arginine and the amino acid substitution at position 207 is serine; the amino acid substitution at position 96 is glutamic acid and the amino acid substitution at position 205 is serine; the amino acid substitutions are at positions 99 and 196, and the amino acid substitution at position 99 is glutamic acid and the amino acid substitution at position 196 is valine; the amino acid substitutions are at positions 99 and 160, and the amino acid substitution at position 99 is glutamic acid and the amino acid substitution at position 160 is cysteine; the amino acid substitutions are at positions 96, 103, and 114, and the amino acid substitution at position 96 is arginine, the amino acid substitution at position 103 is serine, and the amino acid substitution at position 114 is valine; the amino acid substitutions are at positions 96, 103, and 114, and the amino acid substitution at position 96 is glutamic acid, the amino acid substitution at position 103 is serine, and the amino acid substitution at position 114 is valine; the amino acid substitutions are at positions 96, 157, and 202, and the amino acid substitution at position 96 is arginine, the amino acid substitution at position 157 is asparagine, and the amino acid substitution at position 202 is histidine; the amino acid substitutions are at positions 96, 157, and 202, and the amino acid substitution at position 96 is glutamic acid, the amino acid substitution at position 157 is serine, and the amino acid substitution at position 202 is histidine; the amino acid substitutions are at positions 59, 95, and 100, and the amino acid substitution at position 59 is asparagine, the amino acid substitution at position 95 is glutamic acid, and the amino acid substitution at position 100 is alanine; the amino acid substitutions are at positions 59, 95, and 100, and the amino acid substitution at position 59 is asparagine, the amino acid substitution at position 95 is glycine, and the amino acid substitution at position 100 is alanine; the amino acid substitutions are at positions 160, 180, and 198, and the amino acid substitution at position 160 is valine, the amino acid substitution at position 180 is valine, and the amino acid substitution at position 198 is tryptophan; the amino acid substitutions are at positions 70, 91, and 99, and the amino acid substitution at position 70 is valine, the amino acid substitution at position 91 is glutamine, and the amino acid substitution at position 99 is glutamic acid; the amino acid substitutions are at positions 71, 95, and 127, and the amino acid substitution at position 71 is valine, the amino acid substitution at position 95 is glutamic acid, and the amino acid substitution at position 127 arginine; the amino acid substitutions are at positions 71, 95, and 127, and the amino acid substitution at position 71 is valine, the amino acid substitution at position 95 is arginine, and the amino acid substitution at position 127 arginine; the amino acid substitutions are at positions 59, 101, and 194, and the amino acid substitution at position 59 is asparagine, the amino acid substitution at position 101 is histidine, and the amino acid substitution at position 194 glycine.

In a further embodiment, the present invention is directed toward a modified TetR(J) protein comprising a plurality of amino acid substitutions at amino acid positions selected from the group consisting of positions 59, 70, 71, 91, 95, 96, 98, 99, 100, 101, 103, 110, 114, 127, 157, 158, 159, 160, 180, 190, 194, 196, 198, 202, and 207 of the TetR(J) protein as depicted in SEQ ID NO: 40, wherein said modified TetR(J) protein binds a TetR(J) operator sequence with greater affinity in the presence of tetracycline than in the absence of tetracycline. In particular aspects of this embodiment: the amino acid substitution at position 98 is arginine; the amino acid substitution at position 110 is selected from the group consisting of alanine, leucine, valine, proline, phenylalanine and tryptophan; the amino acid substitution at position 157 is selected from the group consisting of glycine, serine, threonine, cysteine, tyrosine, asparagine, and glutamine; the amino acid substitution at position 190 is selected from the group consisting of glycine, serine, threonine, cysteine, tyrosine, glutamine, and asparagine; the amino acid substitution at position 196 is selected from the group consisting of alanine, leucine, valine, proline, phenylalanine, and tryptophan; the amino acid substitution at position 207 is selected from the group consisting of glycine, serine, threonine, cysteine, tyrosine, asparagine, and glutamine; the amino acid substitutions are at positions 96 and 159, and the amino acid substitution at position 96 is arginine and the amino acid substitution at position 159 is leucine; the amino acid substitutions are at positions 96 and 159, and the amino acid substitution at position 96 is glutamic acid and the amino acid substitution at position 159 is leucine; the amino acid substitutions are at positions 96 and 190, and the amino acid substitution at position 96 is arginine and the amino acid substitution at position 190 is glutamine; the amino acid substitutions are at positions 96 and 190, and the amino acid substitution at position 96 is glutamic acid, and the amino acid substitution at position 188 is glutamine; the amino acid substitutions are at positions 96 and 207, and the amino acid substitution at position 96 is arginine and the amino acid substitution at position 207 is serine; the amino acid substitutions are at positions 96 and 207, and the amino acid substitution at position 96 is glutamic acid and the amino acid substitution at position 207 is serine; the amino acid substitutions are at positions 99 and 196, and the amino acid substitution at position 99 is glutamic acid and the amino acid substitution at position 196 is valine; the amino acid substitutions are at positions 99 and 160, and the amino acid substitution at position 99 is glutamic acid and the amino acid substitution at position 160 is cysteine; the amino acid substitutions are at positions 96, 103, and 114, and the amino acid substitution at position 96 is arginine, the amino acid substitution at position 103 is serine, and the amino acid substitution at position 114 is valine; the amino acid substitutions are at positions 96, 103, and 114, and the amino acid substitution at position 96 is glutamic acid, the amino acid substitution at position 103 is serine, and the amino acid substitution at position 114 is valine; the amino acid substitutions are at positions 96, 157, and 202, and the amino acid substitution at position 96 is arginine, the amino acid substitution at position 157 is asparagine, and the amino acid substitution at position 202 is histidine; the amino acid substitutions are at positions 96, 157, and 202, and the amino acid substitution at position 96 is glutamic acid, the amino acid substitution at position 157 is serine, and the amino acid substitution at position 202 is histidine; the amino acid substitutions are at positions 59, 95, and 100, and the amino acid substitution at position 59 is asparagine, the amino acid substitution at position 95 is glutamic acid, and the amino acid substitution at position 100 is alanine; the amino acid substitutions are at positions 59, 95, and 100, and the amino acid substitution at position 59 is asparagine, the amino acid substitution at position 95 is glycine, and the amino acid substitution at position 100 is alanine; the amino acid substitutions are at positions 160, 180, and 198; the amino acid substitution at position 160 is valine, the amino acid substitution at position 180 is valine, and the amino acid substitution at position 198 is tryptophan; the amino acid substitutions are at positions 70, 91, and 99, and the amino acid substitution at position 70 is valine, the amino acid substitution at position 91 is glutamine, and the amino acid substitution at position 99 is glutamic acid; the amino acid substitutions are at positions 71, 95, and 127, and the amino acid substitution at position 71 is valine, the amino acid substitution at position 95 is glutamic acid, and the amino acid substitution at position 127 arginine; the amino acid substitutions are at positions 71, 95, and 127, and the amino acid substitution at position 71 is valine, the amino acid substitution at position 95 is arginine, and the amino acid substitution at position 127 arginine; the amino acid substitutions are at positions 59, 101, and 194, and the amino acid substitution at position 59 is asparagine, the amino acid substitution at position 101 is histidine, and the amino acid substitution at position 194 glycine.

In another embodiment, the present invention is directed toward a modified TetR(Z) protein comprising a plurality of amino acid substitutions at amino acid positions selected from the group consisting of positions 63, 74, 75, 95, 99, 100, 102, 103, 104, 105, 107, 114, 118, 137, 164, 165, 166, 167, 177, 181, 183, 185, 189, and 194 of the TetR(Z) protein as depicted in SEQ ID NO: 41, wherein said modified TetR(Z) protein binds a TetR(Z) operator sequence with greater affinity in the presence of tetracycline than in the absence of tetracycline. In particular aspects of this embodiment: the amino acid substitution at position 102 is histidine; the amino acid substitution at position 114 is selected from the group consisting of alanine, leucine, valine, proline, phenylalanine and tryptophan; the amino acid substitution at position 164 is selected from the group consisting of glycine, serine, threonine, cysteine, tyrosine, asparagine, and glutamine; the amino acid substitution at position 177 is selected from the group consisting of glycine, serine, threonine, cysteine, tyrosine, glutamine, and asparagine; the amino acid substitution at position 183 is selected from the group consisting of alanine, leucine, valine, proline, phenylalanine, and tryptophan; the amino acid substitution at position 194 is selected from the group consisting of glycine, serine, threonine, cysteine, tyrosine, asparagine, and glutamine; the amino acid substitutions are at positions 100 and 166, and the amino acid substitution at position 100 is arginine and the amino acid substitution at position 166 is leucine; the amino acid substitutions are at positions 100 and 166, and the amino acid substitution at position 100 is glutamic acid and the amino acid substitution at position 166 is leucine; the amino acid substitutions are at positions 100 and 177, and the amino acid substitution at position 100 is arginine and the amino acid substitution at position 177 is glutamine; the amino acid substitutions are at positions 100 and 177, and the amino acid substitution at position 100 is glutamic acid and the amino acid substitution at position 177 is glutamine; the amino acid substitutions are at positions 100 and 188, and the amino acid substitution at position 100 is arginine and the amino acid substitution at position 188 is glutamine; the amino acid substitutions are at positions 100 and 188, and the amino acid substitution at position 100 is glutamic acid and the amino acid substitution at position 188 is glutamine; the amino acid substitutions are at positions 100 and 114, and the amino acid substitution at position 100 is arginine and the amino acid substitution at position 114 is phenylalanine; the amino acid substitutions are at positions 100 and 114, and the amino acid substitution at position 100 is glutamic acid and the amino acid substitution at position 110 is phenylalanine; the amino acid substitutions are at positions 100 and 194, and the amino acid substitution at position 100 is arginine and the amino acid substitution at position 194 is serine; the amino acid substitutions are at positions 100 and 194, and the amino acid substitution at position 100 is glutamic acid and the amino acid substitution at position 194 is serine; the amino acid substitutions are at positions 103 and 183, and the amino acid substitution at position 100 is glutamic acid and the amino acid substitution at position 183 is valine; the amino acid substitutions are at positions 103 and 165, and the amino acid substitution at position 103 is glutamic acid and the amino acid substitution at position 165 is cysteine; the amino acid substitutions are at positions 100, 107, and 118, and the amino acid substitution at position 100 is arginine, the amino acid substitution at position 107 is serine, and the amino acid substitution at position 118 is valine; the amino acid substitutions are at positions 100, 107, and 118, and the amino acid substitution at position 100 is glutamic acid, the amino acid substitution at position 107 is serine, and the amino acid substitution at position 118 is valine; the amino acid substitutions are at positions 100, 164, and 189, and the amino acid substitution at position 100 is arginine, the amino acid substitution at position 164 is asparagine, and the amino acid substitution at position 189 is histidine; the amino acid substitutions are at positions 100, 164, and 189, and the amino acid substitution at position 100 is glutamic acid, the amino acid substitution at position 164 is serine, and the amino acid substitution at position 189 is histidine; the amino acid substitutions are at positions 63, 99, and 104, and the amino acid substitution at position 63 is asparagine, the amino acid substitution at position 99 is glutamic acid, and the amino acid substitution at position 104 is alanine; the amino acid substitution at position 59 is asparagine, the amino acid substitution at position 95 is glycine, and the amino acid substitution at position 100 is alanine; the amino acid substitutions are at positions 167, and 185, and the amino acid substitution at position 167 is valine and the amino acid substitution at position 185 is tryptophan; the amino acid substitutions are at positions 74, 95, and 103, and the amino acid substitution at position 74 is valine, the amino acid substitution at position 95 is glutamine, and the amino acid substitution at position 103 is glutamic acid; the amino acid substitutions are at positions 75, 99, and 137, and the amino acid substitution at position 75 is valine, the amino acid substitution at position 99 is glutamic acid, and the amino acid substitution at position 137 arginine; the amino acid substitution at position 71 is valine, the amino acid substitution at position 95 is arginine, and the amino acid substitution at position 137 arginine; the amino acid substitutions are at positions 63, 105, and 181, and the amino acid substitution at position 63 is asparagine, the amino acid substitution at position 105 is histidine, and the amino acid substitution at position 181 glycine.

In a further embodiment, the present invention is directed to a chimeric revTetR protein comprising an amino-terminal DNA-binding domain and a carboxy-terminal tetracycline-binding domain that comprises amino acid residues 50 to 205 of a modified TetR(A) protein comprising an amino acid substitution at a position selected from the group consisting of positions 59, 70, 71, 91, 95, 96, 99, 100, 101, 103, 114, 127, 158, 159, 160, 179, 193, 197, and 201 of the TetR(A) protein as depicted in SEQ ID NO: 34, wherein said modified TetR(A) protein binds a TetR(A) operator sequence with greater affinity in the presence of tetracycline than in the absence of tetracycline.

In a further embodiment, the present invention is directed to a chimeric revTetR protein comprising an amino-terminal DNA-binding domain and a carboxy-terminal tetracycline-binding domain that comprises amino acid residues 50 to 205 of a modified TetR(B) protein comprising an amino acid substitution at a position selected from the group consisting of positions 59, 70, 71, 91, 95, 96, 99, 100, 101, 103, 114, 127, 158, 159, 160, 178, 192, 196, and 200 of the TetR(B) protein as depicted in SEQ ID NO: 36, wherein said modified TetR(B) protein binds a TetR(B) operator sequence with greater affinity in the presence of tetracycline than in the absence of tetracycline.

In a further embodiment, the present invention is directed to a chimeric revTetR protein comprising an amino-terminal DNA-binding domain and a carboxy-terminal tetracycline-binding domain that comprises amino acid residues 50 to 205 of a modified TetR(C) protein comprising an amino acid substitution at a position selected from the group consisting of positions 59, 70, 71, 91, 95, 96, 99, 100, 101, 103, 114, 127, 158, 159, 164, 182, 196, 200, and 204 of the TetR(C) protein as depicted in SEQ ID NO: 38, wherein said modified TetR(C) protein binds a TetR(C) operator sequence with greater affinity in the presence of tetracycline than in the absence of tetracycline.

In a further embodiment, the present invention is directed to a chimeric revTetR protein comprising an amino-terminal DNA-binding domain and a carboxy-terminal tetracycline-binding domain that comprises amino acid residues 50 to 205 of a modified TetR(D) protein comprising an amino acid substitution at a position selected from the group consisting of positions 59, 70, 71, 91, 95, 96, 99, 100, 101, 103, 114, 127, 158, 159, 160, 178, 192, 196, and 200 of the TetR(D) protein as depicted in SEQ ID NO: 40, wherein said modified TetR(D) protein binds a TetR(D) operator sequence with greater affinity in the presence of tetracycline than in the absence of tetracycline.

In a further embodiment, the present invention is directed to a chimeric revTetR protein comprising an amino-terminal DNA-binding domain and a carboxy-terminal tetracycline-binding domain that comprises amino acid residues 50 to 205 of a modified TetR(E) protein comprising an amino acid substitution at a position selected from the group consisting of positions 59, 70, 71, 91, 95, 96, 99, 100, 101, 103, 114, 127, 158, 159, 160, 175, 189, 193, and 197 of the TetR(E) protein as depicted in SEQ ID NO: 42, wherein said modified TetR(E) protein binds a TetR(E) operator sequence with greater affinity in the presence of tetracycline than in the absence of tetracycline.

In a further embodiment, the present invention is directed to a chimeric revTetR protein comprising an amino-terminal DNA-binding domain and a carboxy-terminal tetracycline-binding domain that comprises amino acid residues 50 to 205 of a modified TetR(G) protein comprising an amino acid substitution at a position selected from the group consisting of positions 59, 70, 71, 91, 95, 96, 99, 100, 101, 103, 114, 127, 160, 161, 162, 180, 194, 198, and 202 of the TetR(G) protein as depicted in SEQ ID NO: 44, wherein said modified TetR(G) protein binds a TetR(G) operator sequence with greater affinity in the presence of tetracycline than in the absence of tetracycline.

In a further embodiment, the present invention is directed to a chimeric revTetR protein comprising an amino-terminal DNA-binding domain and a carboxy-terminal tetracycline-binding domain that comprises amino acid residues 50 to 205 of a modified TetR(H) protein comprising an amino acid substitution at a position selected from the group consisting of positions 59, 70, 71, 91, 95, 96, 99, 100, 101, 103, 114, 127, 158, 159, 160, 180, 194, 198, and 202 of the TetR(H) protein as depicted in SEQ ID NO: 46, wherein said modified TetR(H) protein binds a TetR(H) operator sequence with greater affinity in the presence of tetracycline than in the absence of tetracycline.

In a further embodiment, the present invention is directed to a chimeric revTetR protein comprising an amino-terminal DNA-binding domain and a carboxy-terminal tetracycline-binding domain that comprises amino acid residues 50 to 205 of a modified TetR(J) protein comprising an amino acid substitution at an amino acid position selected from the group consisting of positions 59, 70, 71, 91, 95, 96, 99, 100, 101, 103, 114, 127, 158, 159, 160, 180, 194, 198, and 202 of the TetR(J) protein as depicted in SEQ ID NO: 48, wherein said modified TetR(J) protein binds a TetR(J) operator sequence with greater affinity in the presence of tetracycline than in the absence of tetracycline.

In a further embodiment, the present invention is directed to a chimeric revTetR protein comprising an amino-terminal DNA-binding domain and a carboxy-terminal tetracycline-binding domain that comprises amino acid residues 50 to 205 of a modified TetR(Z) protein comprising an amino acid substitution at an amino acid position selected from the group consisting of positions 63, 74, 75, 95, 99, 100, 103, 104, 105, 107, 118, 137, 165, 166, 167, 181, 185, and 189 of the TetR(Z) protein as depicted in SEQ ID NO: 50, wherein said modified TetR(Z) protein binds a TetR(Z) operator sequence with greater affinity in the presence of tetracycline than in the absence of tetracycline.

In a further embodiment, the present invention is directed to a chimeric revTetR protein comprising an amino-terminal DNA-binding domain and a carboxy-terminal tetracycline-binding domain comprising, in which the DNA-binding domain comprises amino acid residues 25-40 of an amino acid sequence selected from the group of amino acid sequences depicted in SEQ ID NO: 34, 36, 38, 40, 42, 44, 46, 48, and 50.

In a still further embodiment, the present invention is directed to a chimeric revTetR protein comprising an amino-terminal DNA-binding domain and a carboxy-terminal tetracycline-binding domain comprising, in which the DNA-binding domain comprises amino acid residues 1-50 of an amino acid sequence selected from the group of amino acid sequences depicted in SEQ ID NO: 34, 36, 38, 40, 42, 44, 46, 48, and 50.

As one of skill in the art would recognize, the DNA sequence to which the chimeric tetracycline repressor protein will bind, for any given construct, will correspond to that DNA sequence recognized by the particular DNA binding domain of the selected TetR repressor protein or other DNA-binding protein that is incorporated into the chimera. Therefore, the DNA sequence bound by a chimeric tetracycline repressor protein of the present invention, can be, but is not limited to, a tet operator sequence corresponding to a Tet A, B, C, D, E, G, H, J, and Z operator sequence. Similarly, in other embodiments of the present invention, the chimeric revTetR protein may bind to sequence other than that of a tetO, including, without limitation, the O_{L} operator of bacteriophage λ where the DNA-binding domain of the chimeric revTetR is derived from the λ CI repressor, or the hixL and/or hixR binding sites where the DNA-binding domain of the chimeric revTetR is derived from the Hin recombinase protein.

Chimeric revTetR proteins therefore may comprise, in one embodiment, an amino terminal DNA binding domain derived from a recombinase selected from the group consisting of Hin, Gin, Cin, and Pin, fused to a carboxy-teraminal tetracycline binding domain of a revTetR protein selected from, but not limited to, the group consisting of a revTetR modified repressor of any one of TetR(A), TetR(B), TetR(C), TetR(D), TetR(E), TetR(G), TetR(H), TetR(J), and TetR(Z) classes. The DNA-binding domain of Hin comprises the 52 carboxy-terminal amino acids of that protein; the DNA-binding domain of Gin comprises the 56 carboxy-terminal amino acids of that protein; the DNA-binding domain of Cin comprises the 51 carboxy-terminal amino acids of that protein; and the DNA-binding domain of Pin comprises the 47 carboxy-terminal amino acids of that protein. The tetracycline -binding domain of a chimeric revTetR protein comprises a revTetR protein lacking the TetO DNA-binding domain, which includes about fifty amino-terminal amino acids. Recombinant genes expressing such chimeric revTetR proteins are prepared according to methods well known in the art, which encode a protein comprising about 50 amino terminal residues corresponding the carboxy terminus of a prokaryotic recombinase such as, but not limited to Hin, Cin, Gin, and Pin, fused to about 150 carboxy-terminal amino acids corresponding to a revTetR protein disclosed herein. As one of ordinary skill would appreciate, minor variations in the amino acid sequence of such chimeric revTetR proteins would be useful in maximizing, or minimizing the binding of such proteins to the sites recognized by the recombinases, i.e. the hixL and hixR sites bound by Hin, the gixL and gixR sites bound by Gin, the cinL and cinR sites bound by Cin, and the pixL and pixR sites bound by Pin recombinase (Feng et al. 1994, Science 263: 348-55). Moreover, one of ordinary skill would appreciate derivatives of such chimeric revTetR proteins having enhanced or diminished binding to one or more of the recombinase binding sites disclosed above, in the presence of tetracycline or a tetracycline analog, may be selected using the methods disclosed herein.

In a further embodiment, the present invention is directed toward chimeric revTetR proteins comprising DNA recognition segments or regions derived from a non-revTetR DNA binding protein combined with a tetracycline binding domain derived from a revTetR protein. In this embodiment, rather than combining an entire DNA-binding domain from a non-revTetR DNA binding protein with a tetracycline binding domain derived from a revTetR protein, only those residues or segments involved in DNA sequence recognition are used to construct the chimeric proteins. In one non-limiting example, a helix-turn-helix motif believed to be intimately involved in DNA sequence recognition by a non-revTetR DNA binding protein is used to replace e.g. the helix-turn-helix motif believed to be intimately involved in TetO recognition in a revTetR DNA binding protein. Chimeric DNA binding proteins constructed in this manner would bind DNA sequences other than a tet operator sequence and would still be subject to tet-regulation as described above. Suitable non-revTetR DNA binding proteins useful in this embodiment include, but are not limited to Hin, Gin, Cin, Pin, and the λ CI repressor protein.

In still another embodiment of the present invention, chimeric revTetR proteins as described above, which have altered DNA-binding traits and are capable of binding to DNA sequences other than a tet operator, are further modified and refined. Such optimization of DNA-binding properties for a particular purpose is carried out using mutagenesis procedures and screening methods as described herein as well as in the art.

5.3 Characterization of Modified Repressors

The modified tetracycline repressors of the present invention are useful for regulating gene expression in a wide variety of prokaryotic organisms. While it is anticipated that each identified revTetR repressor will be broadly applicable across a number of organisms, it is possible that any given revTetR repressor may have slightly different activities from organism to organism, including little to undetectable activity. It is contemplated that one of skill in the art following the teachings provided herein will be able to determine the relative activity of any given revTetR repressor in view of the desired amount of regulation without undue experimentation.

As shown in FIG. 2 and Table 5, the exemplary revTetR repressors (e.g., those set forth in SEQ ID NOS.: 2, 4, 6, 8, 10, 12, 14, 16, 18, 20, 22, 24, 26, 28, 30, as well as representative examples selected from among those set forth in SEQ ID NOS.: 71-264) exhibit the reverse phenotype in a representative prokaryotic organism, Escherichia coli, compared to wild-type repressor, although the absolute level of non-repressed and repressed transcription varies amongst the revTetR repressors. The varied levels of transcriptional regulation advantageously increase the flexibility and range of repressed versus non-repressed levels of regulated gene product. By selecting the appropriate revTetR and tet sequence for use in the methods described herein, repressed and non-repressed levels of the regulated gene may be varied over a wide range as well as the overall ratio of induction.

The relative ratios of non-repressed to repressed levels of transcription for the collection of identified revTetR repressors range from about 1.4-fold to about 50-fold at 28° C. and from about 1.3-fold to 40-fold at 37° C. For example, modified revTetR repressors of the present invention comprising an amino acid substitution of arginine for glycine at position 96 (e.g., SEQ ID NO. 24) repress transcription 19-fold at 37° C. but only to a less extent at 28° C. (5.7-fold, Table 2). Furthermore, modified revTetR repressors of the present invention comprising the arginine for glycine substitution at position 96 and further comprising a substitution or substitutions of serine for threonine at position 103 and valine for glutamic acid at position 114 (e.g., SEQ ID NO. 2); leucine for proline at position 159 (e.g., SEQ ID NO. 6); or glutamine to histidine at position 188 (e.g., SEQ ID NO. 12) have pronouncedly different activities. For instance, the additional substitutions of serine for leucine at position 103 and valine for glutamic acid at position 114 completely abolishes the ability of these revTetR repressors to repress transcription in the presence of tetracycline or tetracycline analog at 37° C. while increasing repression at 28° C. by as much as 2-fold. Thus, the combination of the substitutions at positions 103 and 114 results in a revTetR repressor that is unable to effectively repress transcription at 37° C. demonstrating that the residues at these positions contribute to and/or modulate the reverse phenotype of revTetR repressors in prokaryotic organisms.

The revTetR repressors of the present invention are used to modulate transcription from a prokaryotic promoter operably associated with a tet operator within the range of from about 5° C. to about 60° C., from about 10° C. to about 55° C., from about 15° C. to about 50° C., from about 20° C. to about 45° C., from about 25° C. to about 40° C., an about 28° C. to about 37° C.

Similarly modified revTetR repressors of the present invention comprising an amino acid substitution at position 96 (glutamic acid for glycine) and further comprising a substitution serine for leucine at position 205 (e.g., SEQ ID NO. 14); or phenylalanine for tryptophan at position 110 (e.g., SEQ ID NO. 16) have varying activities. For instance, the resulting modified revTetR repressors have similar activities at 28° C. (36.3-fold v. 33.1-fold) but dramatically different activities at 37° C. (22-fold v. 5-fold). Therefore, the introduction of a substitution of phenylalanine for tryptophan at position 110 may be introduced by one of skill in the art to modulate the activity of the resulting modified revTetR repressor at 37° C., which may be helpful for designing temperature-specific reveTetR repressors (e.g., see Section 5.5.4.1.).

In addition, modified revTetR repressors of the present invention comprising an amino acid substitution of glutamic acid for valine at position 99 (SEQ ID NO. 26) repress transcription 41-fold at 37° C. and 18-fold at 28° C. Modified revTetR repressors of the present invention comprising the glutamic acid for valine at position 99 and further comprising a substitution or substitutions of valine for isoleucine at position 194 (e.g., SEQ ID NO. 18); cysteine for arginine at position 158 (e.g., SEQ ID NO. 20); or valine for alanine at position 70 and glutamine for leucine at position 91 (e.g., SEQ ID NO. 22) also have pronouncedly different activities. For instance, the additional substitution of cysteine for arginine at position 158 increases repression at 28° C. by 50% but reduces the level of repression 5-fold at 37° C. whereas the additional substitution of valine for isoleucine at position 194 increases repression at 28° C. by greater than 2.5-fold but reduces the level of repression 4-fold at 37° C.

Still further, modified revTetR repressors of the present invention comprising amino acid substitutions of asparagine for isoleucine for position 59, glutamic acid for aspartic acid at position 95, and alanine for histidine at position 100 (e.g., SEQ ID NO. 10) repressed transcription at 28° C. and 37° C to a similar extent as the modified revTetR repressors comprising amino acid substitutions arginine for glycine at position 96 and leucine for proline at position 159 (about 9-fold and 20-fold, respectively). In contrast, modified revTetR repressors of the present invention comprising the amino acid substitution of asparagine for isoleucine for position 59, but comprising different substitutions of arginine for lysine at position 98, histidine for leucine at position 101 and glycine for serine at position 192 (e.g., SEQ ID NO. 30) and, valine for alanine at position 71, glycine (GGC) for aspartic acid at position 95, and arginine for leucine at position 127 (e.g., SEQ ID NO. 28) virtually eliminated repression at 28° C. Modified revTetR repressors of the present invention that have substitutions of valine for alanine at position 160, valine for aspartic acid at position 178, tryptophan for glycine at position 196 (e.g., SEQ ID NO. 8) have greatly reduced levels of transcription at 28° C. in the presence or absence of tetracycline or tetracycline analog but relatively wild-type levels of transcription at 37° C., though the ratio of non-repressed to repressed levels of transcription is substantially lower than that of wild-type TetR.

Therefore, one of skill in the art can introduce similar mutations at the corresponding positions in the other classes of tetracycline repressor, or chimera, thereof, based on the teachings herein and the amino acid sequences of the positions provided in Table 3 to generate revTetR repressors in these classes that are useful in the methods described herein.

5.3.1 Temperature-Specific RevTetR Repressors

Modified revTetR proteins that exhibit the reverse phenotype in prokaryotes only at particular temperatures, e.g., exhibit the reverse phenotype only at 28° C. or 37° C., but not both, are also provided. In addition to the revTetR mutations described above that confer a reverse phenotype only at 28° C. (e.g., SEQ ID NO. 2), a substitution at position 96 and additional substitutions of aspargine for aspartic acid at position 157 and histidine for glutamine at position 200 (e.g., SEQ ID NO. 4) also completely eliminate repression at 37° C. resulting in a modified revTetR proteins that exhibit the reverse phenotype in prokaryotes only at 28° C. (See Table 2, FIG. 2).

Conversely, modified revTetR repressors that exhibit the reverse phenotype in prokaryotes only at 37° C. are also provided. For example, modified revTetR repressors comprising amino acid substitutions of asparagine for leucine at position 59, arginine for lysine at position 98, histidine for leucine at position 101 and glycine for serine at position 192 (e.g., SEQ ID NO. 30) and valine for alanine at position 71, glycine (GGC) for aspartic acid at position 95, and arginine for leucine at position 127 (e.g., SEQ ID NO. 28) fail to repress transcription at 28° C.

Therefore, one of skill in the art can introduce similar mutations at the corresponding positions in the other classes of tetracycline repressor, or chimera, thereof, based on the teachings herein and the amino acid sequences of the positions provided in Table 2 to generate temperature-specific revTetR repressors in these classes that are useful in the methods described herein. These temperature-specific revTetR repressors are particularly useful for determining and validating gene products essential for cellular proliferation by comparing expression of the target gene product regulated by the temperature-specific revTetR repressor at repressing and non-repressing conditions.

The tet-regulated expression systems disclosed herein, which comprise at least one revTetR DNA-binding protein, are particularly advantageous in that they enable regulation of gene expression by exposure of the prokaryotic cell to tetracyline, which acts as a co-repressor. Tetracycline is inexpensive, readily penetrates prokaryotic cells, and is used in the present context only at very low, non-antibiotic, levels. Moreover, there are a number of tetracycline analogs available and some, including but not limited to anhydrotetracycline, not only have a greater affinity for TetR, but also are less active as antibiotics. The revTetR-regulated gene expression systems disclosed herein can be established in essentially any prokaryotic cell using an endogenous promoter, where wild-type levels of gene expression are generally maintained in the absence of tetracycline or an analogue thereof.

5.4 Antibodies to Modified Repressors

Described herein are methods for the production of antibodies capable of specifically recognizing epitopes of one or more of the revTetR proteins described above. Such antibodies can include, but are not limited to, polyclonal antibodies, monoclonal antibodies (mAbs), human, humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

It is presumed that a number of the modified revTetR repressors of the present invention will have a conformation that is different from that of wild-type TetR. For the production of antibodies to the altered conformation of the revTetR repressors, various host animals can be immunized by injection with a revTetR protein, or a portion thereof containing one of the amino acid substitutions set forth herein. Such host animals can include but are not limited to rabbits, mice, and rats, to name but a few. Various adjuvants can be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Accordingly, a method of eliciting an immune response in an animal, comprising introducing into the animal an immunogenic composition comprising an isolated revTetR polypeptide, the amino acid sequence of which comprises at least one revTetR substitution and 9 consecutive residues of one of SEQ ID NO: 2, 4, 6, 8, 10, 12, 14, 16, 18, 20, 22, 24, 26, 28, 30, and 71-264.

Polyclonal antibodies are heterogeneous populations of antibody molecules derived from the sera of animals immunized with an antigen, such as a revTetR repressor polypeptide, or an antigenic functional derivative thereof containing one of the amino acid substitutions set forth herein are provided. For the production of polyclonal antibodies, host animals such as those described above, can be immunized by injection with a revTetR repressor polypeptide supplemented with adjuvants as also described above. The antibody titer in the immunized animal can be monitored over time by standard techniques, such as with an enzyme linked immunosorbent assay (ELISA) using immobilized polypeptide. If desired, the antibody molecules can be isolated from the animal (e.g., from the blood) and further purified by well-known techniques, such as protein A chromatography to obtain the IgG fraction.

Monoclonal antibodies, which are homogeneous populations of antibodies to a particular antigen, can be obtained by any technique that provides for the production of antibody molecules by continuous cell lines in culture. These include, but are not limited to the hybridoma technique of Kohler and Milstein, (1975, Nature 256: 495-97; and U.S. Pat. No. 4,376,110), the human B-cell hybridoma technique (Kosbor et al., 1983, Immunology Today 4:72; Cole et al., 1983, Proc. Natl. Acad. Sci. USA 80: 2026-30), and the EBV-hybridoma technique (Cole et al., 1985, Monoclonal Antibodies And Cancer Therapy, Alan R. Liss, Inc., pp. 77-96). Such antibodies can be of any immunoglobulin class including IgG, IgM, IgE, IgA, IgD and any subclass thereof. The hybridoma producing the mAb of this invention can be cultivated in vitro or in vivo. Production of high titers of mAbs in vivo makes this the presently preferred method of production.

Alternative to preparing monoclonal antibody-secreting hybridomas, a monoclonal antibody directed against a revTetR polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP J Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. 5,625,126; U.S. Pat. No. 5,633,425; U.S. Pat. No. 5,569,825; U.S. Pat. No. 5,661,016; and U.S. Pat. No. 5,545,806.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903). For example, human antibodies specific to epitopes responsible for the reverse phenotype of these repressors would be highly desirable for monitoring revTetR in vivo expression levels.

Antibody fragments which recognize specific epitopes can be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries can be constructed (Huse et al., 1989, Science 246:1275-1281) to allow rapid and easy identification of monoclonal Fab fragments with the desired specificity.

Antibodies provided herein may also be described or specified in terms of their binding affinity to a target gene product. Preferred binding affinities include those with a dissociation constant or K_{d} less than 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, or 10⁻¹⁵M.

Antibodies directed against a revTetR repressor polypeptide or fragment thereof containing one of the amino acid substitutions set forth herein can be used diagnostically to monitor levels of a revTetR repressor polypeptide in the tissue of an host as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

5.5 Nucleic Acids Encoding Modified Repressors

Described herein are nucleic acids of the invention which encode the modified tetracycline repressors and chimeric tetracycline repressors of the invention, such as those described in Section 5.2.

In one embodiment, the isolated nucleic acids of the invention comprise nucleotide substitutions that result in codon changes in the TetR (BD) chimera (SEQ ID NO. 32) at amino acid positions 96 or 99, or at positions 96, 103 and 114; positions 96, 157 and 200; positions 96 and 159; positions 160, 178, 196; positions 59, 95 and 100; positions 96 and 188; positions 96 and 205; positions 96 and 110; positions 99 and 194; positions 99 and 158; positions 70, 91 and 99; positions 71, 95 and 127; positions 59, 98, 101 and 192. These nucleic acids encode modified tetracycline repressors that display the reverse phenotype. These nucleic acids can be prepared by modifying a nucleotide sequence that encode the TetR (BD) chimera, such as the nucleotide sequence set forth in SEQ ID NO: 31. The relative activity of these exemplary revTetR repressors encoded by the nucleotide sequences of the invention and wild type TetR repressor at two different assay temperatures is illustrated in FIG. 2, and discussed in detail in Section 5.5.

In particular embodiments, the nucleotide substitution that confers a reverse phenotype in prokaryotic organisms is a change of the glycine codon (GGG) to an arginine codon (AGG) at position 96 (e.g., SEQ ID NO. 23). In addition, isolated nucleic acids comprising the glycine to arginine codon substitution at position 96 and which further comprise codon changes of threonine (ACG) to serine (TCG) at position 103 and glutamic acid (GAA) to valine (GTA) at position 114 (e.g., SEQ ID NO. 1); proline (CCT) to leucine (CTT) at position 159 (e.g., SEQ ID NO. 5); or histidine (CAT) to glutamine (CAA) at position 188 (e.g., SEQ ID NO. 11).

In another embodiment, the nucleotide substitutions that confer a reverse phenotype in prokaryotic organisms are changes of the glycine codon (GGG) to a glutamic acid codon (GAG) at position 96 and which further comprises nucleotide substitutions resulting in codon changes of aspartic acid (GAC) to aspargine (AAC) at position 157 and glutamine (CAG) to histidine (CAT) at position 200 (e.g., SEQ ID NO. 3); leucine (TTG) to serine (TCG) at position 205 (e.g., SEQ ID NO. 13); or tryptophan (TAT) to phenylalanine (TTT) at position 110 (e.g., SEQ ID NO. 15).

In yet another embodiment, the nucleotide substitution that confers a reverse phenotype in prokaryotic organisms is a change of the valine codon (GTG) to a glutamic acid codon (GAG) at position 99 (e.g., SEQ ID NO. 25). In addition, isolated nucleic acids were identified comprising the valine to glutamic acid codon substitution at position 99 and which further comprise nucleotide substitutions that result in codon changes of isoleucine (ATC) to valine (GTC) at position 194 (e.g., SEQ ID NO. 17); arginine (CGC) to cysteine (TGC) at position 158 (e.g., SEQ ID NO. 19); or alanine (GCG) to valine (GTG) at position 70 and leucine (CTG) to glutamine (CAG) at position 91 (e.g., SEQ ID NO. 21).

Furthermore, isolated nucleic acids were identified comprising nucleotide sequences having nucleotide substitutions that result in codon changes of: isoleucine (ATC) to asparagine (AAC) at position 59, aspartic acid (GAC) to glutamic acid (GAA) at position 95, and histidine (CAC) to alanine (GCT) at position 100 (e.g., SEQ ID NO. 9); isoleucine (ATC) to asparagine (AAC) at position 59, lysine (AAA) to arginine (AGA) at position 98, leucine (CTC) to histidine (CAC) at position 101 and serine (AGC) to glycine (GGC) at position 192 (e.g., SEQ ID NO. 29); alanine (GCA) to valine (GTA) at position 160, aspartic acid (GAC) to valine (GTC) at position 178, and glycine (GGG) to tryptophan (TGG) at position 196 (e.g., SEQ ID NO. 7); and, alanine (GCG) to valine (GTG) at position 71, aspartic acid (GAC) to glycine (GGC) at position 95, and leucine (CTG) to arginine (CGG) at position 127 (e.g., SEQ ID NO. 27).

In other preferred embodiments, the isolated nucleic acids comprise a nucleotide sequence that encodes any of the amino acid sequences set forth in SEQ ID NOS. 2, 4, 6, 8, 10, 12, 14, 16, 18, 20, 22, 24, 26, 28, 30, and 71-264. In further embodiments, the isolated nucleic acids comprise the sequence of nucleotides selected from the group consisting of SEQ ID NOS. 1, 3, 5, 7, 9, 11, 13, 15, 17, 19, 21, 23, 25, 27, 29, and 265-458. In other embodiments, the isolated nucleic acids comprise a nucleotide sequence encoding modified revTetR proteins that exhibit the reverse phenotype in prokaryotes only at particular temperatures, e.g., exhibit the reverse phenotype only at 28° C. or 37° C., but not both.

Minor nucleotide substitutions corresponding to regions of the polypeptide coding sequence that are not involved in the reverse phenotype may be introduced without compromising the reverse phenotype and are encompassed within the scope of the invention. For instance, nucleotide substitutions were identified in the revTetR coding region of a number of isolated nucleic acids that did not result in a codon change or alter the reverse phenotype (i.e., a silent mutation), for example, the arginine codon (CGT to CGC) at position 62 (e.g., SEQ ID NO. 29), the serine codon (TCC to TCT) at position 74 (SEQ ID NO. 11), the asparagine codon (AAT to AAC) at position 82, the arginine codon (CGC to CGT) at position 87 (e.g., SEQ ID NO. 5), the valine codon (GTG to GTC) at position 99 (e.g., SEQ ID NO. 9), the proline codon at position 105 (CCT to CCC), and the glycine codon (GGG to GGT) at position 130 (e.g., SEQ ID NO. 27). Accordingly, one of skill in the art based on the teachings and guidance provided herein would be readily able to identify those nucleotide sequences encoding a revTetR repressor comprising minor nucleotide substitutions that do not alter or substantially alter the amino acid sequence of one of the exemplary revTetR repressors.

To isolate homologous revTetR repressors, the revTetR nucleotide sequences and fragments thereof described above can be labeled and used as probes to screen a library of DNA encoding mutant TetR sequences. Hybridization conditions should be of a lower stringency when the cDNA library was derived from a Tet repressor class or chimera different from the class of TetR from which the labeled sequence was derived. For guidance regarding such conditions see, for example, Sambrook et al., 1989, Molecular Cloning, A Laboratory Manual, Cold Spring Harbor Press, N.Y.; and Ausubel et al., 1989, Current Protocols in Molecular Biology, (Green Publishing Associates and Wiley Interscience, N.Y.). In particular, oligonucleotide probes, primers or fragments that comprise nucleotide sequences encompassing the specified nucleotide substitutions described above that confer the reverse phenotype in one class of tetracycline repressor may be used in hybridization reactions or DNA amplification methods to specifically identify those members of the library containing the desired substitutions.

Alternatively, a modified revTetR repressor can be created by site-directed mutagenesis by substitution of amino acid residues in the sequence of a wild type Tet repressor, or chimera thereof. Tables 1 and 3 lists the positions of amino acid residues present in various tetracycline repressor classes at which desirable substitutions can be made, while Table 4 provides the position (column 1) and the amino acid residue found at that position (column 2) for the hybrid TetR(BD) protein in which specific revTetR alleles were identified. The remaining columns provide the amino acid found in the corresponding position for TetR(A), TetR(B), TetR(C), TetR(D), TetR(E), TetR(G), TetR(H), TetR(J), and TetR(Z), in which each residue identical to that found in TetR(BD) is presented in bold.

TABLE 4
Amino acid residues of TetR repressors

| AA | TetR | TetR | TetR | TetR | TetR | TetR | TetR | TetR | TetR | TetR |
|---|---|---|---|---|---|---|---|---|---|---|
| Position | (BD) | (A) | (B) | (C) | (D) | (E) | (G) | (H) | (J) | (Z) |
| 59 | Ile | Met | Met | Met | Ile | Ile | Ile | Met | Ile | Val |
| 70 | Ala | Arg | Leu | Arg | Ala | Leu | Glu | Leu | Leu | Glu |
| 71 | Ala | Ala | Glu | Asp | Ala | Glu | Glu | Pro | Ala | Ser |
| 91 | Leu | Leu | Leu | Leu | Leu | Leu | Leu | Leu | Leu | His |
| 95 | Asp | Asp | Asp | Asp | Asp | Asp | Asp | Asp | Asp | Asp |
| 96 | Gly | Gly | Gly | Gly | Gly | Gly | Gly | Gly | Gly | Gly |
| 98 | Lys | Arg | Lys | Arg | Lys | Arg | Arg | Lys | Lys | Arg |
| 99 | Val | Ile | Val | Ile | Val | Ile | Ile | Ile | Ile | Leu |
| 100 | His | His | His | His | His | His | His | His | His | His |
| 101 | Leu | Ala | Leu | Ala | Leu | Ile | Ala | Ala | Ala | Ala |
| 103 | Thr | Thr | Thr | Thr | Thr | Thr | Thr | Thr | Thr | His |
| 110 | Tyr | Met | Tyr | Met | Tyr | Phe | Phe | Phe | Phe | Asp |
| 114 | Glu | Asp | Glu | Asp | Glu | Glu | Glu | Glu | Glu | Glu |
| 127 | Leu | Ala | Leu | Ala | Leu | Val | Pro | Leu | Leu | Glu |
| 157 | Asp | Glu | Glu | Glu | Asp | Glu | Asp | Glu | Glu | Gly |
| 158 | Arg | Arg | Arg | Arg | Arg | His | Arg | Arg | Arg | Asn |
| 159 | Pro | Gly | Glu | Gly | Pro | Val | Pro | Glu | Glu | Ala |
| 160 | Ala | Thr | Thr | Thr | Ala | Ile | Asp | Lys | Lys | Ser |
| 178 | Asp | Asp | Asp | Tyr | Asp | Ala | Glu | Asp | Asp | None |
| 188 | His | Gln | Phe | Arg | His | Phe | Phe | Phe | Phe | Phe |
| 192 | Ser | Val | Leu | Leu | Ser | Ser | Ser | Val | Val | Ala |
| 196 | Gly | Gly | Gly | Gly | Gly | Gly | Gly | Gly | Gly | Gly |
| 200 | Gln | Arg | Gln | Met | Gln | Gln | Leu | Val | Val | Ser |
| 205 | Leu | Arg | Ser | Arg | Leu | Leu | Leu | His | Lys | Leu |

In still further embodiments, the isolated nucleic acid molecules encode a revTetR repressor comprising a sequence of nucleotides containing a mutation or mutations that confers a reverse phenotype in prokaryotic organisms and preferably having at least 70%, 75%, 80%, 85%, 90%, 95%, 98% or 99% nucleotide sequence identity, more preferably at least 90%, 95%, 98% or 99% sequence identity, to any of the nucleotide sequences set forth in SEQ ID NOS. 1, 3, 5, 7, 9, 11, 13, 15, 17, 19, 21, 23, 25, 27, 29, and 265-458. To determine the percent identity of two sequences, e.g., nucleotide or amino acid, the sequences are aligned for optimal comparison purposes (e.g., gaps can be introduced in the sequence of a first amino acid or nucleotide sequence for optimal alignment with a second amino acid or nucleotide sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul (1990) Proc. Natl. Acad. Sci. U.S.A. 87: 2264-68, modified as in Karlin and Altschul (1993) Proc. Natl. Acad. Sci. U.S.A. 90: 5873-77. Such an algorithm is incorporated into the NBLAST and XBLAST programs of Altschul et al., 1990, J. Mol. Biol. 215: 403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25: 3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., http://www.ncbi.nlm.nih.gov). Another preferred, non-limiting example of a mathematical algorithm utilized for the comparison of sequences is the algorithm of Myers and Miller, (1988) CABIOS 4: 11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The present invention also includes polynucleotides, preferably DNA molecules, that hybridize to the complement of the nucleic acid sequences encoding the modified tetracycline repressors. Such hybridization conditions can be highly stringent or less highly stringent, as described above and known in the art. The nucleic acid molecules of the invention that hybridize to the above described DNA sequences include oligodeoxynucleotides (“oligos”) which hybridize to the nucleotide sequence encoding the revTetR repressor under highly stringent or stringent conditions. In general, for oligos between 14 and 70 nucleotides in length the melting temperature (Tm) is calculated using the formula:

Tm(° C.)=81.5+16.6 (log[monovalent cations (molar)]+0.41 (% G+C)−(500/N)

where N is the length of the probe. If the hybridization is carried out in a solution containing formamide, the melting temperature may be calculated using the equation:

Tm(° C.)=81.5+16.6 (log[monovalent cations (molar)])+0.41 (% G+C)−(0.61) (% formamide)−(500/N)

where N is the length of the probe. In general, hybridization is carried out at about 20-25 degrees below Tm (for DNA-DNA hybrids) or about 10-15 degrees below Tm (for RNA-DNA hybrids). Other exemplary highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos).

In one embodiment, the isolated nucleic acid molecules comprise a sequence of nucleotides containing a revTetR mutation or mutations that hybridize under moderate stringency conditions to the entire length any of the nucleotide sequences set forth in SEQ ID NOS. 1, 3, 5, 7, 9, 11, 13, 15, 17, 19, 21, 23, 25, 27, 29, and 265-458. In still yet another embodiment, the isolated nucleic acid molecules comprise a sequence of nucleotides containing a revTetR mutation or mutations that hybridize under high stringency conditions to the entire length of any of the nucleotide sequences set forth in SEQ ID NOS. 1, 3, 5, 7, 9, 11, 13, 15, 17, 19, 21, 23, 25, 27, 29, and 265-458 are provided. Isolated nucleic acids encoding a full-length complement of the nucleotide sequence any of these nucleic acids are also provided.

In another embodiment, isolated nucleic acid fragments of the revTetR repressor proteins comprising at least 10, 15, 20, 25, 30, 35, 40, 45 or 50 contiguous nucleotides containing at least one mutation encoding conferring a reverse phenotype in prokaryotes, or the complement thereof, are also provided. Particularly preferred nucleic acid fragments are those containing at least one mutation conferring a reverse phenotype in prokaryotic organisms located within nucleotides 210-216, 273 to 309, 330-381, 450-477, or 480 to 605 of SEQ ID NO. 31. Additional nucleic acid fragments are those containing at least one mutation conferring a reverse phenotype in prokaryotic organisms within nucleotide positions 37-75, 40-72, 49-69, 157-183, and 283-297 of SEQ ID NO: 31.

In another embodiment, the invention also encompasses (a) DNA vectors that comprise a nucleotide sequence comprising any of the foregoing sequences encoding a revTetR and/or their complements (including antisense molecules); (b) DNA expression constructs that comprise a nucleotide sequence comprising any of the foregoing sequences encoding a revTetR operably linked with a regulatory element that directs the expression of the coding sequences; and (c) genetically engineered host cells that comprise any of the foregoing sequences of the revTetR gene, including the revTetR gene operably linked with a regulatory element that directs the expression of the coding sequences in the host cells.

Recombinant DNA methods which are well known to those skilled in the art can be used to construct vectors comprising nucleotide sequences encoding a revTetR, and appropriate transcriptional/translational control signals. The various sequences may be joined in accordance with known techniques, such as restriction, joining complementary restriction sites and ligating, blunt ending by filling in overhangs and blunt ligation, Bal31 resection, primer repair, in vitro mutagenesis, or the like. Polylinkers and adapters may be employed, when appropriate, and introduced or removed by known techniques to allow for ease of assembly of the DNA vectors and expression constructs. These methods may also include in vivo recombination/genetic recombination. At each stage of the manipulation of the enzyme gene sequences, the fragment(s) may be cloned, analyzed by restriction enzyme, sequencing or hybridization, or the like. A large number of vectors are available for cloning and genetic manipulation. Normally, cloning can be performed in E. coli. See, for example, the techniques described in Sambrook et al., 1989, Molecular Cloning: A Laboratory Manual, Cold Spring Harbor Press, Cold Spring Harbor, N.Y.; Ausubel, 1989, supra; Methods in Enzymology: Guide to Molecular Cloning Techniques, Academic Press, Berger, S. L. and A. R. Kimmel eds., 1987; Pla et al., Yeast 12:1677-1702 (1996); Kinghorn and Unkles in Aspergillus, ed. by J. E. Smith, Plenum Press, New York, 1994, Chapter 4, p.65-100; which are incorporated by reference herein in their entireties.

In various embodiments of the invention, DNA vectors that comprise a nucleotide sequence encoding a revTetR of the invention, may further comprise replication functions that enable the transfer, maintenance and propagation of the DNA vectors in one or more species of host cells, including but not limited to E. coli cells, Gram positive bacteria, and Gram negative bacteria. The choice of the vector will typically depend on the compatibility of the vector with the host cell into which the vector is to be introduced. The vectors may be linear or closed circular plasmids, cosmid, or phagemids. The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated.

In specific embodiments of the present invention, expression of a revTetR-encoding gene is modulated so as to provide different levels of revTetR protein in a particular host. The level of expression of a gene encoding a particular revTetR protein may be manipulated by the choice of promoters with different transcription rates to which the revTetR coding sequence is operably associated, the inclusion of one or more positive and/or negative regulatory sequences which control the rate of transcription from that promoter, and the copy number of the vector carrying the revTetR coding sequence. Representative, but not limiting examples of each of these elements is provided supra. Therefore, by manipulating each of these elements independently or in a concerted manner, the level of a revTetR protein within the prokaryotic host cell can be precisely established over a wide range.

5.5.1 Identification of Modified Tetracycline Repressors

Isolated nucleic acids of the present invention comprising nucleotide sequences encoding modified tetracycline repressors that exhibit the desired reverse phenotype in prokaryotic organisms may be identified, for example, from amongst a collection of mutated wild type tetracycline repressors using a number of in vitro or cell-based screening techniques, including those described herein. Any method known to those of skill in the art may be used to introduce nucleotide substitutions into the coding sequence of gene encoding a tetracycline repressor protein to create the pool of mutated repressors or portions thereof comprising at least one substitution including, but not limited to, spontaneous mutations, error-prone PCR (Leung et al., (1989) Technique 1: 11-15), chemical mutagenesis (Eckert et al., Mutat. Res. (1987) 178: 1-10), site-directed mutagenesis (Kunkel (1985) Proc. Natl. Acad. Sci. USA 82:488-92; Oliphant et al., (1986) Gene 44: 177-83) or DNA shuffling (Stemmer, (1994), Proc. Natl. Acad. Sci. USA 91: 10747-51).

As described in Example 1, for instance, an isolated nucleic acid comprising the nucleotide sequence encoding the C-terminal portion of TetR(D) can be subjected to DNA shuffling with a nucleic acid encoding the N-terminal portion of TetR(B) to create a pool of isolated nucleic acids encoding modified chimeric TetR(BD) repressors. The pool encoding the modified chimeric TetR(BD) repressors can be cloned and screened in a representative prokaryotic organism, Escherichia coli, for those clones comprising at least one mutation encoding an amino acid substitution and conferring a reverse phenotype. Analogous methods may be employed to create a pool of modified tetracycline repressors for screening using isolated nucleic acids encoding a member of or a chimera of any class of TetR repressor. The reverse phenotype may be identified or confirmed using a number of methods well known to those of skill in the art including, but not limited to, in vitro transcription assays and cell-based assays using reporter systems that are regulated by tetracycline.

A modified revTetR repressor of the present invention can be selected, for example, by incorporating an isolated nucleic acid of the present invention (e.g., see Section 5.2.3) into an expression vector and introduced into the desired prokaryotic organism for screening. A screening assay is used which allows for selection of a revTetR repressor which binds to a tet operator sequence in the prokaryotic organism only in the presence of tetracycline. For example, a pool of mutated nucleic acids in an expression vector can be introduced into the organism in which tet operator sequences control the expression of a reporter gene, e.g., a gene encoding a Lac repressor and the Lac repressor controls the expression of a gene encoding an selectable marker (e.g., drug resistance). Binding of a Tet repressor to tet operator sequences in the bacteria will inhibit expression of the Lac repressor, thereby inducing expression of the selectable marker gene. Cells expressing the marker gene are selected based upon the selectable phenotype (e.g., drug resistance). For wild-type Tet repressors, expression of the selectable marker gene will occur in the absence of tetracycline. A modified revTetR repressor is selected using this system based upon the ability to induce expression of the selectable marker gene in the bacteria only in the presence of tetracycline.

In another embodiment, methods for identifying modified tetracycline repressors that exhibit a reverse phenotype in prokaryotes are provided. In one aspect, the method comprises introducing into a prokaryotic organism a nucleic acid comprising a reporter gene operatively linked to a promoter regulated by tetracycline or tetracycline analog, transforming a culture of the prokaryotic organism with a collection of expression vectors, each comprising a nucleotide sequence encoding a modified tetracycline repressor containing at least one amino acid substitution, expressing the modified tetracycline repressor proteins in the organism in the presence or absence of tetracycline or tetracycline analog, and identifying those transformants that express or express at a higher level the reporter gene in the absence, but not the presence, of the tetracycline or tetracycline analog.

5.5.2 Method of Making Modified Tetracycline Repressors

Described here are methods for preparing recombinant, modified tetracycline repressors that exhibit a reverse phenotype in prokaryotes. Methods of making the modified repressor in a gene regulation system are described in Section 5.6 hereinbelow.

The modified tetracycline repressors or peptides thereof that exhibit a reverse phenotype in prokaryotes of the present invention can be readily prepared, e.g., by synthetic techniques or by methods of recombinant DNA technology using techniques that are well known in the art. Thus, methods for preparing the target gene products of the invention are discussed herein. First, the polypeptides and peptides of the invention can be synthesized or prepared by techniques well known in the art. See, for example, Creighton, 1983, Proteins: Structures and Molecular Principles, W. H. Freeman and Co., N.Y., which is incorporated herein by reference in its entirety. Peptides can, for example, be synthesized on a solid support or in solution.

Alternatively, recombinant DNA methods which are well known to those skilled in the art can be used to construct expressible nucleic acids that encode a modified tetracycline repressor coding sequence such as those set forth in SEQ ID NOS. 1, 3, 5, 7, 9, 11, 13, 15, 17, 19, 21, 23, 25, 27, 29, and 265-458, to which are operably linked the appropriate transcriptional/translational control signals. These methods include, for example, in vitro recombinant DNA techniques, synthetic techniques and in vivo recombination/genetic recombination. See, for example, the techniques described in Sambrook et al., 1989, Molecular Cloning: A Laboratory Manual, Cold Spring Harbor Press, Cold Spring Harbor, N.Y., Pla et al., Yeast 12:1677-1702 (1996), and Ausubel, 1989, supra. Alternatively, RNA capable of encoding target gene protein sequences can be chemically synthesized using, for example, synthesizers. See, for example, the techniques described in Oligonucleotide Synthesis, 1984, Gait, M. J. ed., IRL Press, Oxford, which is incorporated herein by reference in its entirety.

Accordingly, the method for preparing these modified tetracycline repressors comprises introducing into an organism an expressible nucleic acid encoding a modified tetracycline repressor that exhibits a reverse phenotype in the prokaryotic organism, expressing the modified tetracycline repressor in the organism, and purifying the expressed modified tetracycline repressor. In one preferred embodiment, the expressible nucleic acid is an expression vector comprising the nucleotide sequence encoding the modified tetracycline repressor. In another preferred embodiment, the nucleotide sequence encoding the modified tetracycline repressor is selected from nucleotide sequence encoding any of the amino acid sequences of SEQ ID NOS. 2, 4, 6, 8, 10, 12, 14, 16, 18, 20, 22, 24, 26, 28, 30, and 71-264.

A variety of host-expression vector systems can be utilized to express the modified revTetR repressor coding sequences of the invention. Such host-expression systems represent vehicles by which the coding sequences of interest can be produced and subsequently purified, but also represent cells which can, when transformed or transfected with the appropriate nucleotide coding sequences, exhibit the target gene protein of the invention in situ. These include but are not limited to microorganisms such as bacteria (e.g., E. coli, B. subtilis) transformed with recombinant bacteriophage DNA, plasmid DNA or cosmid DNA expression vectors containing target gene protein coding sequences; yeast (e.g., Saccharomyces, Aspergillus, Candida, Pichia) transformed with recombinant yeast expression vectors containing the target gene protein coding sequences; insect cell systems infected with recombinant virus expression vectors (e.g., baculovirus) containing the target gene protein coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing target gene protein coding sequences; or mammalian cell systems (e.g. COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter). If necessary, the nucleotide sequences of coding regions may be modified according to the codon usage of the host such that the translated product has the correct amino acid sequence.

In bacterial systems, a number of expression vectors can be advantageously selected depending upon the use intended for the modified repressor being expressed. For example, when a large quantity of such a protein is to be produced, for the generation of antibodies or to screen for binding to DNA, for example, vectors which direct the expression of high levels of fusion protein products that are readily purified can be desirable. Such vectors include, but are not limited, to the E. coli expression vector pUR278 (Ruther et al., 1983, EMBO J. 2: 1791), in which the target gene protein coding sequence can be ligated individually into the vector in frame with the lacZ coding region so that a fusion protein is produced; pIN vectors (Inouye & Inouye, 1985, Nucleic Acids Res. 13: 3101-09; Van Heeke & Schuster, 1989, J. Biol. Chem. 264: 5503-09); and the like. pGEX vectors can also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene protein can be released from the GST moiety.

Following expression of a modified revTetR repressor, the resulting protein is substantially purified (e.g., see Ettner et al., (1996) J. Chromatogr. 742: 95-105). For example, the expressed proteins may be enriched from culture medium or a cell lysate by salt precipitation (e.g., ammonium sulfate) or gel filtration. The enriched fractions may be further purified using, for example, chromatographic methods, such as affinity chromatography using 1) tet operator sequences bound to solid supports or 2) antibodies directed against revTetR; ion-exchange chromatography or electrophoretic methods such as one- and two-dimensional gel electrophoresis, or isoelectric focusing gels. Such methods for the enrichment or purification of proteins are well known to those of skill in the art (e.g., Sambrook et al., (1989) Molecular Cloning: A Laboratory Manual, Cold Spring Harbor Press, Cold Spring Harbor, N.Y.). For example, revTetR genes are cloned into an expression plasmid such as, but not limited to, pWH1950 (Ettner et al., (1996) J. Chromatogr. 742: 95-105) under the control of a tac promoter, and the recombinant plasmid is used to transform a suitable E. coli host such as E. coli strain RB791. Cells are grown in 3-6 liters of LB medium at 22° C. in flasks on a rotary shaker to a density corresponding to an OD of 0.6 to 1.0. Expression of the recombinant revTetR gene is then initiated by addition of the gratuitous inducer isopropyl-β-D-galactopyranoside to a final concentration of 1 mM. Incubation is continued for 3 to 12 hours and the cells are then collected by centrifugation, resuspended in buffer A (0.05 M NaCl, 2 mM DTT, and 20 mM sodium phosphate, pH 6.8). The resuspended cells are broken by sonication and the revTetR protein purified by cation-exchange chromatography using POROS™ HS/M Medium (Applied Biosystems, Foster City, Calif.) and gel filtration as described, for example by Ettner et al. (Ettner et al., (1996) J. Chromatogr. 742: 95-105). Protein concentration is determined by UV-spectroscopy and saturating fluorescence titrations with anhydrotetracycline. In a specific embodiment, the yield of revTetR is increased by using a richer production medium such as TB-medium, (which is formulated as follows: 12 g tryptone, 24 g yeast extract, and 4 g glycerol are dissolved in distilled water and the volume adjusted to 900 ml. The solution is sterilized by autoclaving and then cooled to 60° C. or less and 100 ml of 0.17 M KH₂PO₄-0.72 M K₂HPO₄, pH 7.4 added), to which 0.4 μM tetracycline is added upon inoculation with the recombinant expression host strain.

5.6 Genetic Regulatory Systems Based on Modified Tet Repressors

Described herein are prokaryotic organisms comprising a system of specific regulation of gene expression that is based on the modified tetracycline repressors of the invention. The regulated gene expression system of the invention comprises a prokaryotic host organism which carries expressible nucleic acid encoding a modified tetracycline repressor of the present invention, and a target gene of which the transcription is to be regulated specifically and which is operatively linked to a promoter and at least one tet operator sequence. In the absence of tetracycline or analogs thereof, wild-type levels of transcription of the target gene operatively linked to the tet operator sequence(s) occur. However, in the presence of tetracycline or analogs thereof, transcription of the target gene is repressed in accordance with the activity of the revTetR present in the prokaryotic organism.

Depending on the revTetR and the operator sequences, the level of repression can vary due to the DNA binding affinity of the revTetR, the affinity of the revTetR for tetracycline or the tetracycline analog used, and/or the ability of the revTetR to block transcription. The level of repression of transcription may vary depending upon the prokaryotic organism and, potentially, the site of integration of the target gene.

Typically, in order to repress transcription of the target gene, the prokaryotic organism is contacted with an effective and sub-lethal amount of tetracycline or a tetracycline analog. For example, to specifically repress target gene expression in a prokaryotic organism in culture, the organism is contacted with tetracycline or an analog thereof by culturing the organism in a medium containing an appropriate concentration of tetracycline or an analog thereof. A preferred concentration range for the inducing agent is between about 10 and about 1000 ng/ml, between about 5 and 1000 ng/ml, and between 1 and 1000 ng/ml. Tetracycline or analogs thereof can be directly added to medium in which the prokaryotic organisms are already being cultured. Alternatively, the cells are harvested from tetracycline-free medium and cultured in fresh medium containing tetracycline, or an analog thereof. Preferably, the prokaryotic organism is cultured in a medium containing a sub-inhibitory concentration of tetracycline or tetracycline analog.

The gene regulation system of the invention can also be used in an animal model wherein the test animal is infected with a prokaryotic organism comprising one or more genes whose expression is regulated by the tet regulatory system of the present invention. To specifically repress prokaryotic gene expression in the such an animal model, the prokaryotic organisms within the animal is contacted with tetracycline or an analog thereof by administering the tetracycline or an analog thereof to the animal. Depending on the animal, the dosage is adjusted to preferably achieve a serum concentration between about 0.05 and 1.0 μg/ml, between about 0.01 and 1.0 μg/ml, and between about 0.005 and 1.0 μg/ml tetracycline or analog thereof. The tetracycline or analog thereof can be administered by any means effective for achieving an in vivo concentration sufficient for the specific regulation of gene expression. Examples of suitable modes of administration include oral administration (e.g., dissolving tetracycline or analog thereof in the drinking water), slow release pellets or implantation of a diffusion pump. Preferably, the animal is a non-human animal, and can include but not limited to non-human primates, mammals such as mouse, rabbits, and rats, and other common laboratory animals.

The ability to use different tetracycline analogs allows for the modulation of the level of expression of a target gene sequence which is linked to a particular tet operator. For example, anhydrotetracycline has been demonstrated to efficiently repress transcription in prokaryotic organisms in the range of about 50-fold (e.g., see FIG. 2). Tetracycline, chlorotetracycline and oxytetracycline have been found generally to be weaker repressing agents.

Thus, an appropriate tetracycline analog can be chosen as a repressing agent based upon the desired level of gene expression. It is also possible to change the level of gene expression in a cell or animal over time by changing the tetracycline analog used as the repressing agent. For example, there may be situations where it is desirable to have a strong repression of target gene expression initially and then have a sustained lower level of target gene expression. Accordingly, an analog that represses transcription effectively can be used initially and then the repressing agent can be switched to tetracycline or an analog that results in a low level of transcription. It is also desirable that, upon removal of tetracycline or tetracycline analog, wild-type levels of transcription can be restored from the regulated target gene, thereby allowing the targeted gene product to be expressed.

Moreover, the gene regulation system of the invention can accommodate regulated expression of more than one target gene. A first target sequence can be regulated by one class of tet operator sequence(s) and a second target sequence is regulated by another class of tet operator sequence. Moreover, chimeric revTet repressors comprising a tetracycline-binding domain from a revTetR protein and a DNA binding domain from a DNA-binding protein other than a TetR protein may be used to regulate one or more genes operably associated with a DNA sequence bound by the non-TetR DNA binding domain of the chimeric protein. Such chimeric proteins would, without limitation, include DNA binding domains that would recognize and bind other operator sequence (e.g, O_{L}, hixL, hixR), with an affinity that can be different than that of a TetR protein for a tet operator sequence. The level of expression of each of the target sequences can be regulated differently and/or independently depending upon which revTetR repressor is used to regulate transcription and which tetracycline analog(s) is used as the repressing agent. Additionally, the expression of each gene may be modulated by varying the concentration of tetracycline or tetracycline analog in the culture medium or within the animal. Thus, the expression system of the invention provides a method not only for turning gene expression on or off, but also for “fine tuning” the level of gene expression at intermediate levels depending upon the type of revTetR, operator sequence, and concentration of agent used.

Different levels of expression of two genes regulated by the same revTet repressor of the present invention can be achieved by operably associating each target gene with a different tet operator sequence. There is sufficient cross-recognition of the different tet operators by individual revTetR proteins (Klock et al. 1985 J. Bacteriol. 161(1): 326-32) to permit a given revTetR protein to regulate the expression of both genes, but to a different extent, at a given concentration of tetracycline or tetracycline analog.

In further embodiments of the present invention, variant revTetR proteins are constructed and those capable of binding to one or more tet operators are identified. In certain embodiments, binding is evaluated against variant tet operators that are not recognized or bound by wild-type TetR proteins. Such variant revTetR proteins are generated by mutagenesis directed toward DNA sequences encoding amino acid residues known to be involved in tet operator sequence recognition. Methods for the generation and evaluation of such variant TetR and, therefore, revTetR proteins and evaluating the affinity with which they bind to naturally-occurring and variant tet operator sequences are well known in the art. See, for example, Baumeister et al. (J. Mol. Biol. 226(4): 1257-70 (1992)), Helbl et al. (J. Mol. Biol. 245(5): 538-48; J. Mol. Biol. 276(2): 313-18 (1998), and J. Mol. Biol. 276(2): 319-24 (1998)), each of which is hereby incorporated by reference in its entirety. Use of such variant revTetR proteins in conjunction with variant tet operator sequences enables separate, tetracyline-dependent regulation of more than one gene within the same prokaryotic cell. By independently varying the level of expression of each of a plurality of revTetR proteins expressed in a prokaryotic cells, wherein each revTetR protein (or variant thereof) recognizes a different tet operator sequence (or variant thereof), the level of expression of each target gene operatively associated with a different tet operator is also independently regulated by the level of tetracycline to which that prokaryotic cell is exposed. Independent regulation of the level of expression of the plurality of revTetR-encoding genes is accomplished, for example, by operatively associating each revTetR-encoding gene with a different promoter which may include additional genetic regulatory elements, such as but not limited to, a repressor or activator binding sequence. In addition, the revTetR-encoding genes may be incorporated within distinct replicons that have different copy numbers within the prokaryotic host cell. Heterodimers between and among different revTetR and/or TetR proteins do not form where the tet-operator binding domains are different for each revTetR and/or TetR protein. Accordingly, each gene that is regulated by a different tet operator can be differentially regulated using different revTetR and/or TetR proteins, where each recognizes and binds a different tet operator.

In a further embodiment, a target gene within a prokaryotic host cell is operatively associated with a tet operator sequence recognized and bound by a wild-type TetR protein as well as a revTetR protein. The prokaryotic host cell further comprises at least one copy of a gene encoding the wild-type TetR protein as well at least one copy of a gene encoding the revTetR protein. In this embodiment, the TetR and revTetR encoding genes are operatively associated with different genetic regulatory elements providing independent expression of each type of repressor protein. In this manner, the target gene is either positively or negatively regulated by the presence of tetracycline, depending on whether the wild-type TetR or the revTetR protein is being expressed, respectively.

In another aspect of the present invention, a prokaryotic structural gene encoding either a positive regulator or a negative regulator of gene expression is engineered to be operably associated with a promoter and at least one tet operator sequence. In this embodiment, the level of expression of the positive or negative regulatory protein (and, consequently each of the genes subject to their regulation) is dependent upon the level of revTet repressor protein in the cell and the concentration of tetracycline or tetracycline analog to which the prokaryotic host is exposed. In this embodiment, addition of tetracycline will result in the binding of a revTetR-tetracycline complex to a tet operator or tet operators and, in one example, thereby repress expression of a negative regulator, leading to increased expression of those genes regulated by the negative regulator. Similarly addition of tetracycline will result in revTetR-mediated repression of the expression of a positive regulator operably associated with a tet operator, thereby leading to decreased expression of those genes regulated by the positive regulator. Where desired, the tet regulatory system of the present invention could therefor be used to regulate expression of both a positive and negative regulatory proteins in the same prokaryotic host, thereby providing a method for simultaneously increasing the expression of one set of co-regulated genes while decreasing the level of expression of a second set of co-regulated genes, by contacting the host expressing a revTetR of the present invention with tetracycline or a tetracycline analog.

5.6.1 Prokaryotic Organisms of the Invention

In various embodiments, prokaryotic organisms comprising an expressible nucleic acid encoding a modified tetracycline repressor of the present invention are provided. Presently preferred prokaryotic organisms for use herein include, but are not limited to Bacillus anthracis, Bacteriodes fragilis, Bordetella pertussis, Burkholderia cepacia, Camplyobacter jejuni, Chlamydia pneumoniae, Chlamydia trachomatus, Clostridium botulinum, Clostridum tetani, Clostridium perfringens, Clostridium difficile, Corynebacterium diptheriae, Enterobacter cloacae, Enterococcus faecalis, Escherichia coli, Haemophilus influenzae, Helicobacter pylori, Klebsiella pneumoniae, Listeria monocytogenes, Moraxella catarrhalis, Mycobacterium leprae, Mycobacterium tuberculosis, Neisseria gonorrhoeae, Nesseria meningitidis, Nocardia asteroides, Proteus vulgaris, Pseudomonas aeruginosa, Salmonella typhi, Salmonella typhimurium, Shigella boydii, Shigella dysenteriae, Shigella flexneri, Shigella sonnei, Staphylococcus aureus, Staphylococcus epidermidis, Streptococcus mutans, Streptococcus pneumoniae, Treptonema pallidum, Vibrio cholerae, Vibrio parahemolyticus, and Yersina pestis. Also included are other related genera and species that cause a disease with substantially similar pathology as that caused by the above prokaryotic organisms.

Any method known to those of skill in the art, including those described herein, may be used to introduce the nucleic acids of the present invention into prokaryotic organisms. Suitable methods for introducing isolated nucleic acids into host cells are known to those of skill in the art and include, but are not limited to, natural competency, calcium chloride transformation, protoplast transformation, electroporation, conjugation, and generalized and specialized transduction (e.g., see Sambrook et al. (1989) Molecular Cloning: A Laboratory Manual, 2nd Edition, Cold Spring Harbor Laboratory press; Gotz et al., (1987) FEMS Microbiol. Lett. 40:285-288; Biswas et al., (1993) J. Bacteriol. 175:3628-3635; Luchansky et al., (1988) Mol. Microbiol. 2, 637-646; Dunny et al., (1991) Appl. Environ. Microbiol. 57, 1194-1201; Cruz-Rodz and Gilmore (1990) Mol. Gen. Genet. 224: 152-154; Park and Stewart (1990) Gene 94: 129-132; Jacob and Hobbs (1974) J. Bacteriol. 117: 360-3721; Novick et al., (1986) J. Mol. Biol. 192: 209-20, and other laboratory textbooks, such as Clark & Russell, Molecular Biology, made simple and fun, Cache River Press, Vienna, Ill.). 5.6.2. Expression Vectors for Expression of Tet Repressors of the Invention

In various embodiments, the present invention provides expressible nucleic acids for the synthesis of the revTet repressors of the present invention which comprise nucleotide sequences encoding a modified tetracycline repressor of the present invention operably linked to another nucleotide sequence that comprises a promoter that is active in the prokaryotic organism(s) of choice. The expressible nucleic acid can be an expression vector, which may propagate extra-chromosomally. Many such expression vectors are known in the art. The promoter may be constitutive or inducible. A wide variety of promoters that are active in gram positive and/or gram negative bacteria are known to those of skill in the art and can be used herein, including but not limited to, the Bacillus aprE and nprE promoters (U.S. Pat. No. 5,387,521), the bacteriophage lambda P_{L} and P_{R} promoters (Renaut, et al., (1981) Gene 15: 81), the trp promoter (Russell, et al., (1982) Gene 20: 23), the tac promoter (de Boer et al., (1983) Proc. Natl. Acad. Sci. USA 80: 21), B. subtilis alkaline protease promoter (Stahl et al. (1984) J. Bacteriol. 158: 411-18) alpha amylase promoter of B. subtilis (Yang et al., (1983) Nucleic Acids Res. 11: 237-49) or B. amyloliquefaciens (Tarkinen, et al., (1983) J. Biol. Chem. 258: 1007-13), the neutral protease promoter from B. subtilis (Yang et al., (1984) J. Bacteriol. 160: 15-21), T7 RNA polymerase promoter (Studier and Moffatt (1986) J Mol Biol. 189(1): 113-30), B. subtilis xyl promoter or mutant tetR promoter active in bacilli (Geissendorfer & Hillen (1990) Appl. Microbiol. Biotechnol. 33: 657-663), Staphylococcal enterotoxin D promoter (Zhang and Stewart (2000) J. Bacteriol. 182(8): 2321-25), cap8 operon promoter from Staphylococcus aureus (Ouyang et al., (1999) J. Bacteriol. 181(8): 2492-500), the lactococcal nisA promoter (Eichenbaum (1998) Appl Environ Microbiol. 64(8): 2763-9), promoters from in Acholeplasma laidlawii (Jarhede et al., (1995) Microbiology 141 (Pt 9): 2071-9), porA promoter of Neisseria meningitidis (Sawaya et al., (1999) Gene 233: 49-57), the fbpA promoter of Neisseria gonorrhoeae (Forng et al., (1997) J. Bacteriol. 179:3047-52), Corynebacterium diphtheriae toxin gene promoter (Schmitt and Holmes (1994) J. Bacteriol. 176(4): 1141-49), the hasA operon promoter from Group A Streptococci (Alberti et al., (1998) Mol Microbiol 28(2): 343-53) and the rpoS promoter of Pseudomonas putida (Kojic and Venturi (2001) J. Bacteriol. 183: 3712-20). All of the above-identified references are incorporated herein by references in their entireties.

By adjusting the strength of the promoter operatively linked to the isolated nucleic acid comprising a nucleotide sequence encoding a revTetR, adjusting the nucleotide sequence of the encoded revTetR repressor to optimize or diminish the use of preferred codons in the prokaryotic host of choice or to stabilize or destabilize the encoding mRNA, and/or adjusting the copy number of the vector backbone, the relative levels of transcription and/or translation of a gene operatively linked to a tet operator sequence(s) may be titrated over a wide range.

5.6.3. Operator Sequences used in Tet-Regulated Expression Systems of the Invention

The genetic regulatory system disclosed herein comprises one or more tet operator sequences, generally two or more, operably associated with the target gene to be controlled by a revTetR of the present invention in the presence of tetracycline or a tetracycline analog. Nucleotide sequences comprising a tet operator sequence recognized and bound by TetR(A), TetR(B), TetR(C), TetR(D), and TetR(E), are provided herein as SEQ ID NO: 51 to 55, respectively. Each of these sequences has been found within the nucleic acid sequence situated between the TetA gene and the TetR gene of each class. Accordingly, although the tet operator sequences specifically recognized by TetR(G), TetR(H), TetR(J), TetR(K), and TetR(Z), have not been precisely defined by genetic analysis, it is apparent that the nucleotide acid sequence situated between the TetA gene and the TetR gene of each of these classes where TetR expression is auto-regulated and TetA expression is tetracycline-inducible, comprises a tet operator sequence as well. One or more of each of these tet operator sequences is operably associated with the target gene using methods well known in the art to provide a chimeric gene that is expressed at reduced level in the presence of a revTetR of the present invention and tetracycline or an analogue thereof.

In another embodiment of the present invention, the revTetR protein is a chimeric protein comprising a tetracycline-binding domain of a TetR protein operably fused to a DNA binding domain derived from a DNA-binding protein other than a TetR protein. In this aspect of the invention, the nucleic acid sequence operably associated with the target gene comprises the nucleotide sequence recognized and bound by the non-TetR-DNA-binding domain of, for example, Hin recombinase. In this example the operator sequence comprises, e.g. the HixL sequence; that is, the operator sequence that operably associated with the target gene comprises SEQ ID NO: 60. The non-TetR-DNA-binding domain may be derived from the DNA-binding domain of Hin recombinase or from the Hin-related proteins, Cin, Gin, and Pin, (SEQ ID NO: 56 to 59, respectively) and the operator sequence operably associated with the target gene will comprise the nucleotide sequences recognized by these recombinases (60-67), or to any one of the group comprising (SEQ ID NO: 60-67) (Feng et al. 1994 Science 263: 348-55).

In certain embodiments of the tet-regulated expression of the present invention, the class of revTetR and corresponding operator sequence are matched with the organism or genus in which they were discovered. For example, a tet-regulated expression system to be established in a prokaryotic organism harboring the pAG1, a gram-positive organism, a member of the genus Corynebacteria including but not limited to Corynebacterium glutamicum, would comprise a revTetR(Z) and tet(Z) operator sequence.

5.7 Uses of the Gene Regulation System

5.7.1 Identification and Validation of Essential Genes

Methods for identifying and validating genes or gene products essential for proliferation or pathogenicity of a prokaryotic organism are provided. In one embodiment, the present invention is directed toward a method for identifying a gene or gene product essential for proliferation or pathogenicity of a prokaryotic organism comprising placing a nucleic acid comprising a nucleotide sequence encoding a putative essential gene under control of at least one tet operator, introducing an expression vector comprising a nucleotide sequence encoding a modified tetracycline repressor into the a prokaryotic organism, expressing the modified tetracycline repressor polypeptide, contacting the organism with a concentration of tetracycline or tetracycline analog sufficient to repress the expression level of gene product, and determining the viability of the organism. In preferred aspects of this embodiment, the concentration of tetracycline or tetracycline analog sufficient to repress the expression level of gene product is a sub-inhibitory concentration.

In one embodiment of the cell-based assays, conditional-expression prokaryotic strains expressing a revTetR repressor described herein, in which the nucleotide sequences required for survival, growth, proliferation, virulence, or pathogenicity of a prokaryotic organism are under the control of a tet regulatable promoter, are grown in the presence of a concentration of tetracycline or analog, or repressor thereof which causes the function of the gene products encoded by these sequences to be rate limiting for growth, survival, proliferation, virulence, or pathogenicity. To achieve that goal, a growth inhibition dose curve of tetracycline or tet analog or repressor is calculated by plotting various doses of tetracycline or repressor against the corresponding growth inhibition caused by the limited levels of the gene product required for fungal proliferation. From this dose-response curve, conditions providing various growth rates, from 1 to 100% as compared to tetracycline or tet analog or repressor-free growth, can be determined. For example, if the regulatable promoter is repressed by tetracycline, the conditional-expression strain may be grown in the presence of varying levels of tetracycline. For example, the highest concentration of the tetracycline or tet analog or repressor that does not reduce the growth rate significantly can be estimated from the dose-response curve. Cellular proliferation can be monitored by growth medium turbidity via OD measurements. In another example, the concentration of tetracycline or tet analog or repressor that reduces growth by 25% can be predicted from the dose-response curve. In still another example, a concentration of tetracycline or tet analog or repressor that reduces growth by 50% can be calculated from the dose-response curve. Additional parameters such as colony forming units (cfu) are also used to measure cellular growth, survival and/or viability.

Conditional-expression cells as described above, which comprise a revTetR according to the present invention, that are to be assayed, are exposed to the above-determined concentrations of tetracycline or tet analog. The presence of the tetracycline or tet analog and the revTetR at this sub-lethal, preferably sub-inhibitory, concentration reduces the amount of the proliferation-required gene product to the lowest level that will support growth of the cells. Cells grown in the presence of this concentration of tetracycline or tet analog or repressor are therefore specifically more sensitive to inhibitors of the proliferation-required protein or RNA of interest as well as to inhibitors of proteins or RNAs in the same biological pathway as the proliferation-required protein or RNA of interest but not specifically more sensitive to inhibitors of unrelated proteins or RNAs.

Prokaryotic cells pretreated with sub-inhibitory concentrations of tetracycline or tet analog or repressor, which therefore contain a reduced amount of proliferation-required target gene product, are used to screen for compounds that reduce cell growth. The sub-lethal concentration of tetracycline may be any concentration consistent with the intended use of the assay to identify candidate compounds to which the cells are more sensitive than are control cells in which this gene product is not rate-limiting. For example, the sub-lethal concentration of the tetracycline or tet analog may be such that growth inhibition is at least about 5%, at least about 8%, at least about 10%, at least about 20%, at least about 30%, at least about 40%, at least about 50%, at least about 60% at least about 75%, at least 80%, at least 90%, at least 95% or more than 95%. Cells which are pre-sensitized using the preceding method are more sensitive to inhibitors of the target protein because these cells contain less target protein to inhibit than wild-type cells.

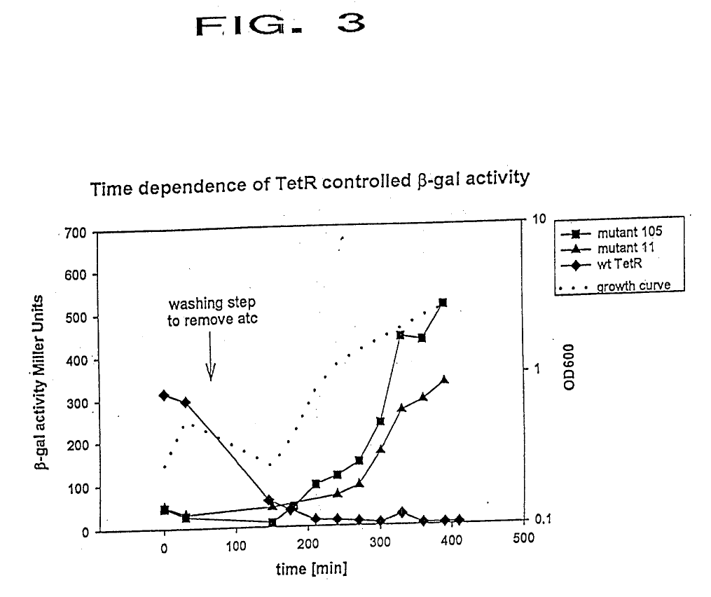

Alternatively, the regulatory system may be utilized to differentiate between a static or cidal phenotype of a putative essential gene product. For example, a prokaryotic organism of the present invention may be incubated in the presence of an inhibitory concentration of tetracycline or analog thereof sufficient to fully repress transcription of the putative essential gene product under the control of at least one tet operator sequence. The tetracycline or analog is then removed by washing whereupon after a predetermined period of time transcription from the tet-regulated promoter is initiated (e.g., see FIG. 3). In those organisms wherein the deprivation of an essential gene product exhibits a “static” phenotype, the organisms will begin to grow as sufficient levels of gene product accumulate to sustain proliferation. In those organisms wherein the deprivation of an essential gene product exhibits a “cidal” phenotype, the organisms will not grow even if sufficient levels of gene product accumulate to sustain proliferation.

It will be appreciated that similar methods may be used to identify compounds which inhibit virulence or pathogenicity. In such methods, the virulence or pathogenicity of cells exposed to the candidate compound which express rate limiting levels of a gene product involved in virulence or pathogenicity is compared to the virulence or pathogenicity of cells exposed to the candidate compound in which the levels of the gene product are not rate limiting. Virulence or pathogenicity may be measured using the techniques described herein.

Similarly, the above method may be used to determine the pathway on which a test compound, such as a test antibiotic acts. A panel of cells, each of which expresses a rate limiting amount of a gene product required for fungal survival, growth, proliferation, virulence or pathogenicity where the gene product lies in a known pathway, is contacted with a compound for which it is desired to determine the pathway on which it acts. The sensitivity of the panel of cells to the test compound is determined in cells in which expression of the nucleic acid encoding the gene product required for proliferation, virulence or pathogenicity is at a rate limiting level and in control cells in which expression of the gene product required for proliferation, virulence or pathogenicity is not at a rate limiting level. If the test compound acts on the pathway in which a particular gene product required for proliferation, virulence, or pathogenicity lies, cells in which expression of that particular gene product is at a rate limiting level will be more sensitive to the compound than the cells in which gene products in other pathways are at a rate limiting level. In addition, control cells in which expression of the particular gene required for fungal proliferation, virulence or pathogenicity is not rate limiting will not exhibit heightened sensitivity to the compound. In this way, the pathway on which the test compound acts may be determined.

In certain aspects of each of these embodiments, regulation of the target gene of a prokaryotic organism (e.g. an essential gene or virulence gene) operatively associated with a tet operator, is modulated, in part, by the level of revTetR protein in the cell. Expression levels of revTetR protein withing a prokaryotic host cell are varied and modulated by the choice of the promoter operatively associated with the structural gene encoding the revTetR protein. Further control over the level of RevTetR expression is obtained by incorporating, or example, one or more regulatory sequences recognized and bound by a repressor protein and/or by an activator protein, and/or one or more sequences recognized and bound by at least one regulatory protein responding to the presence or absence of particular metabolites or substrates, such as but not limited to, glucose and phosphate. An additional level of control over the intracellular level of a RevTetR protein is provided by the copy number of the replicon carrying the revTetR-encoding gene, which can be integrated into the genome of the prokaryotic host or it may be included within a plasmid having high (˜50 to ˜100 or more copies/cell), intermediate (˜10 to ˜50 copies/cell), or low (˜1 to ˜10 copies/cell) copy number.

5.7.2 Target Evaluation in an Animal Model System

Validation of an essential drug target in prokaryotic organisms is often demonstrated by examining the effect of gene inactivation under standard laboratory conditions. Putative drug target genes deemed nonessential under standard laboratory conditions may be examined within an animal model, for example, by testing the pathogenicity of a strain having a deletion in the target gene versus wild type. However, essential drug targets are precluded from animal model studies. Therefore, the most desirable drug targets are omitted from the most pertinent conditions to their target evaluation.

In an embodiment of the invention, conditional expression, provided by the revTetR regulatory system, overcomes this longstanding limitation to target validation within a host environment. Animal studies can be performed using mice inoculated with conditional-expression prokaryotic strains and examining the effect of gene inactivation by conditional expression. Exemplary mouse models for monitoring the bacterial infections include, but are not limited to, the CD-1 mouse model (Yanke et al., (2000) Can J Microbiol. 10: 920-26), peritonitis/sepsis model (e.g., Frimodt-Moller et al., in Handbook of Animal Models of Infection (Zak and Sande eds), Chapter 14, pp. 125-136, Academic Press, San Diego, Calif.) or the murine thigh infection model (e.g., Gudmundsson and Erlendsdottir, in Handbook of Animal Models of Infection (Zak and Sande eds), Chapter 15, pp. 137-144, Academic Press, San Diego, Calif.).

In a preferred embodiment of the invention, the effect on mice injected with a lethal inoculum of a conditional-expression pathogenic prokaryotic organism could be determined depending on whether the mice were provided with an appropriate concentration of tetracycline to inactivate expression of a drug target gene. The lack of expression of a gene demonstrated to be essential under laboratory conditions can thus be correlated with prevention of a terminal infection. In this type of experiment, only mice “treated” with tetracycline-supplemented water, are predicted to survive infection because inactivation of the target gene has killed the conditional-expression prokaryotic pathogen within the host.

5.7.3. Use of revTetR Regulated Genes for Large-Scale Production of Proteins

In certain embodiments, the present invention is directed toward the large-scale protein production using revTetR-regulated gene expression of a target gene product in a prokaryotic host organism. In one aspect of this embodiment, a target gene encoding the protein of interest is operatively associated with a suitable promoter and at least one tetracycline operator sequence such that tet-operator-bound repressor inhibits transcription of the target gene. In one aspect of this embodiment, either or both of the gene encoding a revTetR repressor protein and the gene encoding the target protein are integrated into the genome of the prokaryotic host organism or carried on an episomal replicon in the prokaryotic host cell. Expression of the revTetR protein is regulated or constitutive as desired or required by the adverse or toxic effect of the target gene product on the prokaryotic organism. The level of expression of the revTetR protein is also regulated by the copy number of the replicon carrying the revTetR protein-encoding gene. In certain embodiments, the prokaryotic host cell is grown in the presence of a repressing amount of tetracycline, and at a desirable time, expression of the target gene is induced by removal or reduction of the level of tetracyline or tetracycline analogue by centrifugation, washing, and resuspension of the host cells, by dilution of the host cells into a tetracycline-free medium, or removal of tetracycline or tetracyline analogue by resin binding.

In another aspect of this embodiment, the method uses a revTetR protein that expresses the revTet phenotype only at a low temperature, e.g. 28° C. but not at 37° C. The host cell is cultured at 28° C. in the presence of tetracycline or a tetracycline analog and when desired, expression of the target gene is induced by shifting the host cell culture to 37° C. In yet another aspect of this embodiment, the method uses a revTetR protein that expresses the revTet phenotype only at a high temperature, e.g. 37° C. but not at 28° C. In this embodiment, the prokaryotic host cell is cultured at 37° C. in the presence of tetracycline or a tetracycline analog, and expression of the target gene is induced by shifting the host cell culture to 28° C.

5.7.4 Use of revTetR Regulated Genes in Proteomics

In a further embodiment, the present invention is directed toward the use of revTetR regulated systems for regulation of gene expression in a prokaryotic organism for the analysis of total protein expression in that host. In various aspects of this embodiment, the level of expression of one or more tet-regulated genes is modulated by virtue of the concentration of tetracyline, the level of expression of the revTetR protein, and/or as disclosed in Section 5.7.3, the temperature. In one aspect of this embodiment, one or more genes, which may be essential genes or genes required for pathogenicity or virulence of a prokaryotic organism are operatively associated with at least one tetracycline operator within a host cell expression a revTetR protein of the present invention. The construction of such host cells is carried out according to the methods of Section 5.7.1. Examination of such cells by procedures and methods of proteomics research well known in the art is applied to identify coordinately expressed/regulated proteins and, ideally, the regulatory proteins involved. In one aspect of this embodiment, identified repressors and positive regulators are placed under tet-regulated expression and the nature of the coordinately-regulated expression system is examined, with respect to whether it is essential for survival of the prokaryotic organism and/or required for pathogenicity.

5.7.5 Use of revTetR Regulated Genes for the Expression of Antisense RNA Synthesis

In a further embodiment, expression of one or more target genes in a prokarytoic organism is modulated via tet-regulated expression of an antisense RNA molecule that inhibits translation of mRNA transcribed from the target gene(s). In this embodiment, a coding region encoding a target-gene-specific antisense RNA is operatively associated with a promoter and a tetracycline operator sequence in such a manner that binding of a tetracycline repressor to that operator prevents synthesis of the antisense RNA molecule in the prokaryotic host cell. In various aspects of this embodiment, the level of expression of an antisense RNA molecule, and translation of a target gene mRNA inhibited by the antisense RNA molecule, is modulated by the concentration of tetracyline or its analog, the level of expression of the revTetR protein, and/or the temperature as disclosed in Section 5.7.3.

For example, in the presence of tetracyline, the expression of a target gene is uninhibited in a prokaryotic host cell carrying a tet-regulated antisense RNA coding sequence which is specific for the target gene, and at least one revTetR-encoding gene, since the expression of antisense RNA is inhibited. However, in the absence of tetracycline, the expression of a target gene is inhibited in a prokaryotic host cell carrying a tet-regulated antisense RNA coding sequence which is specific for the target gene, and at least one revTetR-encoding gene, since the expression of the antisense RNA is permitted. In a particular aspect of this embodiment, the target gene corresponds to one copy of a duplicated gene in a prokaryotic organism, thereby allowing the construction of a prokaryotic host cell that can be functionally haploid for that gene product. Such organisms are particularly useful for the detection of anti-microbial agents active against the encoded target gene product.

5.8 Kits

The present invention is further directed toward kits comprising components of the tetracycline-regulated expression systems disclosed herein, and instructions for use thereof Such kits include a recombinant expression vector that encodes at least one revTetR protein operably associated with a promoter active in the prokaryotic host into which the present tet-regulatory system is to be introduced. In another embodiment, the expression vector comprises a structural gene encoding a revTetR protein of the present invention, and an upstream restriction site, generally as part of a polylinker sequence, into which the end user can insert any promoter of interest to that user.

In another embodiment, the kit further comprises a second recombinant expression vector, comprising at least one TetO sequence bracketed by at least two restriction sites positioned on opposite sides of the operator sequence. The end user can insert a promoter into one of these sites and a structural gene encoding a protein (or an antisense RNA molecule) to be placed under tetracycline regulation into the second site. In other embodiments, the second expression vector may comprise a promoter already operably associated with the operator sequence. In still another embodiment, the operator sequence is not a TetO sequence but, rather, corresponds to a binding site for a non-TetR DNA-binding protein which is bound by the DNA binding domain of a chimeric revTetR protein as disclosed herein.

In a further embodiment, the kit may also comprise at least one tetracycline or tetracycline analogue, such as, but not limited to anhydrotetracycline and doxycycline.

5.9 Identification of Non-Antibiotic Inducers of Modified Repressors

In yet another embodiment of the present invention, the modified revTetR repressors may be used in methods for identifying non-antibiotic compounds that specifically interact with revTetR, but not wild type repressors, in prokaryotes. In one embodiment, a method for identifying non-antibiotic compounds that specifically interact with revTetR in a prokaryotic organism is provided, said method comprising introducing into a prokaryotic organism a first nucleic acid comprising a reporter gene operatively linked to a promoter regulated by tetracycline or tetracycline analog, introducing an expression vector comprising a nucleotide sequence encoding a modified tetracycline repressor into the prokaryotic organism, expressing the modified tetracycline repressor, contacting the prokaryotic organism with a plurality of candidate compounds, and identifying those compounds that repress expression of the reporter gene product.

The candidate compounds can be obtained from a number of commercially available sources and include, for example, combinatorial libraries, natural product libraries, peptides, antibodies (including, but not limited to polyclonal, monoclonal, human, humanized, anti-idiotypic, chimeric or single chain antibodies, and FAb, F(ab′)₂ and FAb expression library fragments, and epitope-binding fragments thereof), and small organic or inorganic molecules.

### 6. EXAMPLES

6.1 Construction and Identification of Modified Tetracycline Repressors Exhibiting a Reverse Phenotype in Gram Negative Bacteria

A pool of mutated Tet repressor proteins was generated by a series of steps based on a method described in Stemmer, 1994, Proc. Natl. Acad. Sci. USA 91: 10747-51. Briefly, a double-stranded DNA substrate comprising a nucleotide sequence encoding amino acids 51-208 of TetR(D) (e.g., nucleotides 151 to 624 of SEQ ID NO. 31) was amplified by error-prone PCR (i.e. PCR performed in the presence of 0.5 mM MnCl₂ and unequal concentrations of the four dNTP substrates to introduce random mutations) using Taq DNA polymerase purchased from Pharmacia. Approximately 2-4 μg DNA substrate was digested using about 0.0015 units of DNase I per μl in 100 μl of a solution of 50 mM Tris-HCl, pH 7.4 and 1 mM MgCl₂ for about 10 minutes at room temperature. The DNAse concentration and the duration of the DNAse digestion are determined empirically and adjusted to generate products in the range of about 10 to about 70 bp, as measured by PAGE in an 8% polyacrylamide gel. DNA fragments of about 10 to 70 bp were purified from an 8% polyacrylamide gel as described in Sambrook et al. (Sambrook et al., 2001, Molecular Cloning, A Laboratory Manual, Cold Spring Harbor Press, Cold Spring Harbor, N.Y.). Briefly, a polyacrylamide block containing DNA fragments of the desired size is incubated overnight at 37° C. in PAA-elution buffer, (0.3 M sodium acetate, pH 5.2, 0.01 M MgCl2, and 0.1% SDS). DNA in the eluate is precipitated in ethanol:acetone (1:1).

The nucleic acid molecules comprising the nucleotide sequence of the C-terminal portion of TetR(D) (amino acids 51 to 208), which included random mutations, were assembled from the gel-purified fragments using PCR amplification in the absence of exogenous oligonucleotide primers. For this purpose, the purified, randomized C-terminal TetR(D) fragments were resuspended in PCR mixture (0.2 mM each dNTP, 2.2 mM MgCl₂, 50 mM KCl, 10 mM Tris-HCl, pH 9.0, 0.1% Triton X-100) at a concentration of 10-30 ng/μl and Taq DNA polymerase was added to the reaction mixture (2.5 Units/100 μl). This PCR amplification was followed by a third PCR amplification in the presence of the oligonucleotide primers that had already been used in the error-prone PCR to amplify the reassembled TetR gene. The PCR reactions were carried out in a GeneAmp PCR System 2400 instrument (Perkin-Elmer, Norwalk, Conn.), employing three separate programs. In the first, error-prone PCR amplification was carried out as follows: 30 cycles of 1 min. at 94° C., 1 min. at 55° C., 1 min. at 72° C. The second program, designed to reassemble the tetR gene and incorporate the mutations created in the first program, was performed as follows: 25 cycles of 30 sec. at 94° C., 30 sec. at 30° C., 30 sec. at 72° C. The third program involved PCR amplification in the presence of primers: 25 cycles of 30 sec. at 94° C., 30 sec. at 50° C., 30 sec. at 72° C. The amplified DNA was digested with restriction enzymes that cleave in the termini of the amplified fragments.

The pool of mutated Tet repressors was cloned into plasmid pWH1411 (Baumeister et al., 1992, Proteins: Struct. Funct. Genet. 14: 168-77), which carries a TetR(B) gene, to provide a TetR(BD) chimera that included, as the amino-terminal portion, amino acid residues 1 to 50 of the TetR(B) gene and, as the carboxyl-terminal portion, amino acid residues 51 to 208 of the TetR(D) gene. The resulting plasmid pool was screened in a genetic assay which positively selects for a functional interaction between a Tet repressor and its cognate operator using E. coli strain WH207(λWH25) (the construction of this strain is described in detail in Wissmann et al., (1991) Genetics 128: 225-32). In this E. coli strain, tet operators direct the expression of divergently arranged β-galactosidase (lacZ) and Lac repressor (lacI) genes and the lac regulatory region directs the expression of a galactokinase (galK) gene on plasmid pWH414. Binding of Tet repressors to tet operators turns off transcription of the lacI and lacZ genes. The absence of Lac repressor allows for expression of the galK gene, which enables the E. coli strain to use galactose as a sole carbon source, which serves as one marker. The lacZ⁻ phenotype serves as a second marker. Thus, bacteria containing Tet repressors which bind to tet operators, have a Gal⁺, lacZ⁻ phenotype. Bacteria containing wild-type Tet repressors have a Gal⁺, lacZ⁻ phenotype in the absence of tetracycline. Modified “reverse” Tet repressors (revTetR) were selected based upon a Gal⁺, lacZ⁻ phenotype in the presence of tetracycline.

A total of 15 clones exhibiting a reverse phenotype in E. coli were identified using the above-described screening procedure. The nucleotide and amino acid sequence of the identified revTetR repressors were determined (ABI DNA Sequencer, Perkin Elmer, Norwalk, Conn.) and are shown in SEQ ID NOS: 1, 3, 5, 7, 9, 11, 13, 15, 17, 19, 21, 23, 25, 27 and 29 (nucleotide positions 1-624) and 2, 4, 6, 8, 10, 12, 14, 16, 18, 20, 22, 24, 26, 28 and 30 (amino acid positions 1-208), respectively. The clone designation, identified amino acid substitutions, and relative activity of non-repressed to repressed levels of transcription at two different temperatures are shown in Table 5.

TABLE 5
Relative Activity of revTetR repressors

| Substitution(s) | Clone | 28° C. ratio | 37° C. ratio |
|---|---|---|---|
| GR96, TS103, EV114 | 14 | 24.5 | 1.3 |
| GE96, DN157, QH200 | 17b | 38.5 | 1.4 |
| GR96, PL159 | 5a | 9.5 | 18.4 |
| AV160, DV178, GW196 | 10 | 2.6 | 6.5 |
| IN59, DE95, HA100 | 17a | 9.3 | 23.4 |
| AV71, DG95, LR127 | 19 | 1.4 | 18.9 |
| IN59, KR98, LH101, SG192 | 105 | 2.5 | 30.8 |
| GR96, HQ188 | 7 | 21.2 | 5.3 |
| GE96, LS205 | 9a | 36.3 | 22.2 |
| GE96, YF110 | 9b | 33.1 | 4.2 |
| VE99, IV194 | 15 | 49.6 | 11.2 |
| VE99, RC158 | 20e | 24.3 | 8.2 |
| AV70, LQ91, VE99 | 21g | 32.8 | 6.9 |
| GR96 | 4b | 5.7 | 19.0 |
| VE99 | 11 | 18.1 | 41.1 |

The identified substitutions are listed in Table 5 above using the standard one letter amino acid designation of the wild type amino acid residue, followed by the substituted amino acid residue and the corresponding amino acid position. Thus, for example, clone 14 comprises three amino acid substitutions: an arginine for glycine substitution at position 96 (G96R), a serine for threonine at position 103 (T103S) and valine for glutamic acid at position 114 (G114V; SEQ ID NO. 2).

The ability of each revTetR clone to bind to its cognate tet operator sequence and regulate transcription in a prokaryotic organism, Escherichia coli, in the presence and absence of a tetracycline analog (anhydrotetracycline, atc) was determined (Table 3, FIG. 2). The relative ratios of non-repressed to repressed levels of transcription for the 15 clones range from about 1.4-fold to about 50-fold at 28° C. and from about 1.3-fold to 40-fold at 37° C. For example, clone 4b comprising an amino acid substitution of glutamic acid for glycine at position 96 (e.g., SEQ ID NO. 24) repressed transcription 19-fold at 37° C. but to a less extent at 28° C. (5.7-fold, Table 3). Furthermore, clones 14, 5a and 7 comprising the arginine for glycine substitution at position 96 and further comprising a substitution or substitutions of serine for threonine at position 103 and valine for glutamic acid at position 114; leucine for proline at position 159; or glutamine to histidine at position 188, respectively, have pronouncedly different activities. For instance, the additional substitutions of serine for leucine at position 103 and valine for glutamic acid at position 114 completely abolishes the ability of these revTetR repressors to repress transcription in the presence of tetracycline or tetracycline analog at 37° C. while increasing repression at 28° C. by as much as 2-fold.

Similarly clones 9a and 9b comprising an amino acid substitution at position 96 (glutamic acid for glycine) and further comprising a substitution serine for leucine at position 205 or phenylalanine for tryptophan at position 110, respectively, have varying activities. For instance, clones 9a and 9b have similar activities at 28° C. (36.3-fold v.33.1-fold) but dramatically different activities at 37° C. (22-fold v. 5-fold). Therefore, the introduction of a substitution of phenylalanine for tryptophan at position 110 modulates the activity of the resulting modified revTetR repressor at 37° C.

In addition, clone 11 comprising an amino acid substitution of glutamic acid for valine at position 99 (SEQ ID NO. 26) repressed transcription 41-fold at 37° C. and 18-fold at 28° C.; however, clones 15, 20e, and 21g comprising the glutamic acid for valine at position 99 and further comprising a substitution or substitutions of valine for isoleucine at position 194; cysteine for arginine at position 158; or valine for alanine at position 70 and glutamine for leucine at position 91, respectively, also have pronouncedly different activities. For instance, the additional substitution of cysteine for arginine at position 158 increases repression at 28° C. by 50% but reduces the level of repression 5-fold at 37° C. whereas the additional substitution of valine for isoleucine at position 194 increases repression at 28° C. by greater than 2.5-fold but reduces the level of repression 4-fold at 37° C.

Still further, clone 17a comprising amino acid substitutions of asparagine for isoleucine for position 59, glutamic acid for aspartic acid at position 95, and alanine for histidine at position 100 (e.g., SEQ ID NO. 10) repressed transcription at 28° C. and 37° C. to a similar extent as clone 5a comprising amino acid substitutions arginine for glycine at position 96 and leucine for proline at position 159 (about 9-fold and 20-fold, respectively). In contrast, clone 105 comprising the amino acid substitution of asparagine for isoleucine for position 59, but comprising different substitutions of arginine for lysine at position 98, histidine for leucine at position 101 and glycine for serine at position 192 (e.g., SEQ ID NO. 30) and, valine for alanine at position 71, glycine (GGC) for aspartic acid at position 95, and arginine for leucine at position 127 (e.g., SEQ ID NO. 28) exhibited little to no repression at 28° C. Clone 14 comprising amino acid substitutions of valine for alanine at position 160, valine for aspartic acid at position 178, tryptophan for glycine at position 196 (e.g., SEQ ID NO. 8) had greatly reduces levels of transcription at 28° C. in the presence or absence of tetracycline or tetracycline analog but relatively wild-type levels of transcription at 37° C., though the ratio of non-repressed to repressed levels of transcription was substantially lower than that of wild-type TetR.

6.2. Construction, Identification, and Use of Modified Tetracycline Repressors Exhibiting a Reverse Phenotype in Gram Positive Bacteria

Construction Identification, and Use of revTetR Repressors in Bacillus subtilis

A pool of mutated Tet repressors is created as in Example 6.1 and cloned into an expression vector comprising a promoter active in Bacillus subtilis, such as but not limited to the xyl-operon promoter of Bacillus, expression may be regulated by particular carbon source, such as xylose, or in other embodiments, maltose. Alternatively, each of the nucleotide sequences of SEQ ID NOS. 1, 3, 5, 7, 9, 11, 13, 15, 17, 19, 21, 23, 25, 27, 29, and 265-458 is operatively associated with a promoter active in Bacillus subtilis, and the recombinant gene expressing a revTetR protein so produced is introduced into Bacillus subtilis to establishing the revTetR phenotype in this host. In preferred embodiments, the promoter active in Bacillus subtilis is regulated by a carbon source selected from the group consisting of xylose and maltose.

The revTetR phenotype is determined, in certain embodiments, by analyzing the expression of a reporter gene selected from the group consisting of lacZ, GFP, and luxA, that is under the control of a promoter active in Bacillus subtilis, which promoter has been engineered to comprise at least one tetracycline operator sequence. Accordingly, expression of such indicator genes is repressed by a revTetR repressor in the presence of subinhibitory levels of tetracycline, anhydrotetracycline or other suitable tetracycline analogue. In alternative instances, a direct selection, rather than a screen, is established to allow the isolation of the revTetR mutants in Bacillus subtilis using the strategy described above in Section 6.1. For example, an antibiotic resistance gene, such as a gene encoding kanamycin resistance, can be placed under the control of a negative-regulatory element, such as a repressor protein. The repressor protein, in turn is operatively associated with one or more tet operators such that expression of the repressor results in sensitivity of the host cell to, e.g., kanamycin, in the presence of a wild-type TetR protein in the absence of sub-inhibitory levels of tetracycline, anhydrotetracycline, or other suitable tetracycline analog. In this embodiment, revTetR mutants are selected as kanamycin-resistant in the absence of tetracycline, anhydrotetracycline, or other suitable tetracycline analog, and the revTetR phenotype confirmed by demonstrating kanamycin-sensitivity in the presence of sub-inhibitory levels of tetracycline, anhydrotetracycline, or other suitable tetracycline analog.

Exemplary promoters, which are active in gram positive organisms, such as Bacillus subtilis and that have been modified so as to be placed under tetR regulation include those promoters that have been described (Geissendorfer & Hillen (1990) Appl. Microbiol. Biotechnol. 33: 657-63) as well as the Cad8 operon promoter engineered to contain one or more tet operators.

In certain embodiments, either one or both of the gene encoding the revTetR repressor and the gene encoding the tetracycline-regulated indicator gene are integrated, for example, into the att site in Bacillus subtilis using bacteriophage Φ11 or, alternatively, integrated into the chromosome via homologous recombination into a specified gene (e.g., amiA gene; see Brucker 1997 FEMS Microbiol. Lett. 151(1): 1-8; Biswas et al. 1993 175(11): 3628-35). In certain other embodiments, either or both of the gene encoding the revTetR repressor and the gene encoding the tetracycline regulated indicator gene are maintained episomally. Both may be episomal and carried on different replicons where the plasmids are compatible and different selectable markers are used. Such recombinant nucleic acids are introduced into Bacillus subtilis or other gram-positive prokaryotic organisms by electroporation, using methods known to those of ordinary skill in the art.

For example, where the reporter gene expresses β-galactosidase (lacZ), revTetR-encoding genes may be identified using the screen disclosed in EXAMPLE 6.1. Recombinant DNA can be isolated from the identified organisms, and the sequences encoding the revTetR repressors can be determined by methods known in the art.

Suitable plasmids that may be used for molecular cloning in Bacillus subtilis include chimeric derivatives of plasmids pUB110, pE194, and pSA0501, which encode resistance to kanamycin, erythromycin, and streptomycin, respectively have been described (Gryczan et al. 1980, J. Bacteriol. 141(1): 246-53; Gryczan et al. 1978, J. Bacteriol. 134(l): 318-29; Gryczan et al. 1978 Proc. Natl. Acad. Sci. U.S.A. 75(3): 1428-32). Methods for the direct, positive selection of recombinant plasmids in Bacillus subtilis have also been described, which are based upon pBD124, which encodes resistance to chloramphenicol as well as a wild type thyA protein which confers trimethoprim-sensitivity upon a thyA-thyB Bacillus subtilis host. Accordingly, transformed Bacillus subtilis thyA-thyB host cells carrying a revTetR gene inserted into the Thy gene of pBD124 are selected as resistant to erythromycin and trimethoprim. Other expression systems that are used for expression of revTetR genes in Bacillus subtilis are adapted from those described in U.S. Pat. No. 4,801,537, 4,920,054, and 6,268,169 B1 by removal, or non-incorporation of peptide secretion signals to allow intracellular expression of the encoded revTetR proteins.

Tet-regulated expression of potential target genes/essential genes in Bacillus subtilis is achieved in one non-limiting example, by allele replacement based upon homologous recombination between non-replicating episomal DNA carrying a tet-operator-regulated essential gene bracketed by DNA sequences found upstream and downstream of the target chromosomal gene. Exemplary target genes include, but are not limited to, rpoA, rpoB, gyrA, gyrB, fabG, fabI, and fusA.

Construction, Identification, and Use of revTetR Repressors in Staphylococcus aureus

A pool of mutated Tet repressors is created as in Example 6.1 and cloned into an expression vector comprising a promoter active in Staphylococcus aureus, such as the xyl-operon promoter of Bacillus, expression may be regulated by particular carbon source (xyl/mal). Alternatively, each of the nucleotide sequences of SEQ ID NOS. 1, 3, 5, 7, 9, 11, 13, 15, 17, 19, 21, 23, 25, 27, 29, and 265-458 is operatively associated with a promoter active in Staphylococcus aureus, and the recombinant gene expressing a revTetR protein so produced is introduced into Staphylococcus aureus to confirm the revTetR phenotype in this host. In preferred embodiments, the promoter active in Staphylococcus aureus is regulated by a carbon source selected from the group consisting of xylose and maltose.

The revTetR phenotype is determined, in certain embodiments, by analyzing the expression of a reporter gene selected from the group consisting of lacZ, GFP, and luxA, that is under the control of a promoter active in Staphylococcus aureus, which promoter has been engineered to comprise at least one tetracycline operator sequence. Accordingly, expression of such indicator genes is repressed by a revTetR repressor in the presence of subinhibitory levels of tetracycline, anhydrotetracycline or other suitable tetracycline analogue. In alternative instances, a direct selection, rather than a screen, is established to allow the isolation of the revTetR mutants in Staphylococcus aureus using the strategy described above in Section 6.1. For example, an antibiotic resistance gene, such as a gene encoding kanamycin resistance, can be place under the control of a negative-regulatory element, such as a repressor protein. The repressor protein, in turn is operatively associated with one or more tet operators such that expression of the repressor results in sensitivity of the host cell to, e.g., kanamycin, in the presence of a wild-type TetR protein in the absence of sub-inhibitory levels of tetracycline, anhydrotetracycline, or other suitable tetracycline analog. In this embodiment, revTetR mutants are selected as kanamycin-resistant in the absence of tetracycline, anhydrotetracycline, or other suitable tetracycline analog, and the revTetR phenotype confirmed by demonstrating kanamycin-sensitivity in the presence of sub-inhibitory levels of tetracycline, anhydrotetracycline, or other suitable tetracycline analog.

Exemplary promoters, which are active in gram positive organisms, such as Staphylococcus aureus and that have been modified so as to be placed under tetR regulation include those promoters that have been described (Geissendorfer & Hillen (1990) Appl. Microbiol. Biotechnol. 33: 657-63) including the phage T5 promoter engineered to contain one or more tet operators.

In certain embodiments, either one or both of the gene encoding the revTetR repressor and the gene encoding the tetracycline-regulated indicator gene are integrated, for example, into the chromosome via homologous recombination into a specified gene (e.g., amiA gene) or any non-essential gene. In certain other embodiments, either or both of the gene encoding the revTetR repressor and the gene encoding the tetracycline regulated indicator gene are maintained episomally. Both may be episomal and carried on different replicons where the plasmids are compatible and different selectable markers are used. Such recombinant nucleic acids are introduced into Staphylococcus aureus or other gram-positive prokaryotic organisms by electroporation, using methods known to those of ordinary skill in the art.

For example, where the reporter gene expresses β-galactosidase (lacZ), revTetR-encoding genes may be identified using the screen disclosed in EXAMPLE 6.1. Recombinant DNA can be isolated from the identified organisms, and the sequences encoding the revTetR repressors can be determined by methods known in the art.

Suitable plasmids that may be used for molecular cloning in Staphylococcus aureus include chimeric derivatives of plasmids pUB110, pC194, and pT181, which encode resistance to kanamycin+chloramphenicol, chloramphenicol, and tetracycline, respectively. Derivatives of these molecules have been described (Gryczan et al. 1980, J. Bacteriol. 141(1):246-53; Gryczan et al. 1978, J. Bacteriol. 134(1): 318-29; Gryczan et al. 1978 Proc. Natl. Acad. Sci. U.S.A. 75(3): 1428-32). Plasmid pT181 is a naturally-occurring Staphylococcus aureus plasmid that has a copy number of about 20 and belongs to the incompatibility group Inc3. This plasmid has been sequenced and shown to have 4,437 bp (Khan et al. 1983, Plasmid 10: 251-59). Plasmid pUB110 is a Staphylococcus aureus plasmid having a molecular weight of about 3×10⁶ daltons, and encodes resistance to kanamycin and chloramphenicol (Keggins et al. 1978, Proc. Natl. Acad. Sci. U.S.A. 75: 1423-27; Zyprian et al. 1983, Plasmid 10: 145-59). Plasmid pC194 is a low-molecular weight plasmid (about 2×10⁶ daltons) encoding chloramphenicol resistance, that replicates in Bacillus subtilis as well as in Staphylococcus aureus.

Recombinant DNA molecules are introduced into Staphylococcus aureus strains by transformation using, for example, electroporation. Suitable Staphylococcus aureus host strains include, but are not limited to RN450, RN4220 and N315.

Tet-regulated expression of potential target genes/essential genes in Staphylococcus aureus is achieved in one non-limiting example, by allele replacement based upon homologous recombination between non-replicating episomal DNA carrying a tet-operator-regulated essential gene bracketed by DNA sequences found upstream and downstream of the target chromosomal gene. In other instances, plasmid vectors that replicate only at low temperature by a rolling-circle model are integrated into the Staphylococcus aureus genome at high temperature (37° C.) to form integrants. The temperature is lowered to induce rolling-circle replication leading to excision of the integrated plasmid and, ultimately loss of the plasmid and allele replacement in which a plasmid-borne (recombinant) copy of a gene is substituted for the wild-type genomic copy of that gene (Brucker 1997 FEMS Microbiol. Lett. 151(1): 1-8; Biswas et al. 1993 175(11): 3628-35). In this manner, a wild-type target gene, which may be an essential gene and/or a gene required for virulence or pathogenicity, is replaced with a recombinant gene comprising one or more tet operators functionally associated with that gene. Accordingly, expression of the gene required for virulence or pathogenicity is modulated by the presence of a revTetR repressor protein combined with sub-inhibitory levels of tetracycline, anhydrotetracycline or other suitable tetracycline-like molecule. Expression of the target gene is repressed to a low level, for example, to provide as test strain that is extraordinarily sensitive to inhibitors of the product encoded by the target gene. Exemplary target genes include, but are not limited to, rpoA, rpoB, gyrA, gyrB, fabG, fabI, and fusA.

Construction Identification, and Use of revTetR Repressors in Enterococcus faecalis

A pool of mutated Tet repressors is created as in Example 6.1 and cloned into an expression vector comprising a promoter active in Enterococcus faecalis, such as the Lactococcus lactis P59 promoter, the Enterococcus bacA promoter, the Lactococcal nisin promoter (PnisA), and the pheromone-inducible prgX promoter (Bae et al. 2000, J. Mol. Biol. 297: 861-79). Each of these promoters can be genetically engineered to include one or more tetracycline operators, providing a tetracycline-regulated derivative thereof. Alternatively, each of the nucleotide sequences of SEQ ID NOS. 1, 3, 5, 7, 9, 11, 13, 15, 17, 19, 21, 23, 25, 27, 29, and 265-458, is operatively associated with a promoter active in Enterococcus faecalis, such as those provided above, and the recombinant gene expressing a revTetR protein so produced is introduced into Enterococcus faecalis to confirm the revTetR phenotype in this host. In preferred embodiments, the promoter active in Enterococcus faecalis is regulated; for example, the level of transcription from a prgX promoter, which can be induced by pheromones (Bae et al. 2000, J. Mol. Biol. 297: 861-79).

The revTetR phenotype is determined, in certain embodiments, by analyzing the expression of a reporter gene selected from the group consisting of lacZ, GFP, and luxA, that is under the control of a promoter active in Enterococcus faecalis, which promoter has been engineered to comprise at least one tetracycline operator sequence. Accordingly, expression of such indicator genes is repressed by a revTetR repressor in the presence of subinhibitory levels of tetracycline, anhydrotetracycline or other suitable tetracycline analogue. In alternative instances, a direct selection, rather than a screen, is established to allow the isolation of the revTetR mutants in Enterococcus faecalis using the strategy described above in Section 6.1. For example, an antibiotic resistance gene, such as a gene encoding kanamycin resistance, can be place under the control of a negative-regulatory element, such as a repressor protein. The repressor protein, in turn is operatively associated with one or more tet operators such that expression of the repressor results in sensitivity of the host cell to, e.g., kanamycin, in the presence of a wild-type TetR protein in the absence of sub-inhibitory levels of tetracycline, anhydrotetracycline, or other suitable tetracycline analog. In this embodiment, revTetR mutants are selected as kanamycin-resistant in the absence of tetracycline, anhydrotetracycline, or other suitable tetracycline analog, and the revTetR phenotype confirmed by demonstrating kanamycin-sensitivity in the presence of sub-inhibitory levels of tetracycline, anhydrotetracycline, or other suitable tetracycline analog.

Exemplary promoters, which are active in the gram negative organism, Enterococcus faecalis, are modified so as to be placed under tetR regulation; examples of such exemplary promoters are provided above, and each of these promoters can be engineered to include one or more tet operators to provide a tetracycline-regulated promoter that can be operatively associated with a target or indicator gene of interest.

In certain embodiments, either one or both of the gene encoding the revTetR repressor and the gene encoding the tetracycline-regulated indicator gene are integrated into the Enterococcus faecalis chromosome via homologous recombination. In certain other embodiments, either or both of the gene encoding the revTetR repressor and the gene encoding the tetracycline regulated indicator gene are maintained episomally. Both may be episomal and carried on different replicons where the plasmids are compatible and different selectable markers are used. Plasmid vectors useful for recombinant DNA expression and gene transfer in Enterococcus faecalis include but are not limited to Enterococcus/E. coli shuttle vectors, such as those based upon pAM401 (e.g. pMGS100 and pMGS101; Fujimoto et al. 2001, Appl. Environ. Microbiol. 67: 1262-67), vectors comprising the nisin promoter (Bryan et al. 2000, Plasmid, 44: 183-90 (Eichenbaum et al. 1998, Appl. Environ. Microbiol. 64: 2763-69), and conjugative plasmids, such pCF10, which comprises a pheromone-inducible tetracycline resistance gene (Chung et al. 1995, J. Bacteriol. 177: 2107-17; also see Manganelli et al. 1998 FEMS Microbiol. Lett. 168(2): 259-68; Chaffin et al. 1998, Gene 219(1-2): 91-99; and Poyart et al. 1997 FEMS Microbiol Lett. 1562(2): 193-98). Such recombinant nucleic acids are introduced into Enterococcus faecalis or other gram-positive prokaryotic organisms by electroporation, using methods known to those of ordinary skill in the art (e.g. Manganelli et al. 1998 FEMS Microbiol. Lett. 168(2): 259-68). Suitable markers useful for selection in Enterococcus faecalis include, but are not limited to, tetracycline resistance, kanamycin resistance, erythromycin resistance, and streptomycin resistance. Appropriate Enterococcus faecalis host strains include, but are not limited to OG1RF, which is described in Dunny et al. (Dunny et al. 1981, Plasmid 6: 270-78). One example of a suitable growth medium for propagation of Enterococcus faecalis is Todd-Hewitt Broth (THB) (see Dunney et al. 1985, Proc. Natl. Acad. Sci. U.S.A. 82: 8582-86).

For example, where the reporter gene expresses β-galactosidase (lacZ), revTetR-encoding genes may be identified using the screen disclosed in EXAMPLE 6.1. Recombinant DNA can be isolated from the identified organisms, and the sequences encoding the revTetR repressors can be determined by methods known in the art.

Tet-regulated expression of potential target genes/essential genes in Enterococcus faecalis is achieved in one non-limiting example, by allele replacement based upon homologous recombination between non-replicating episomal DNA carrying a tet-operator-regulated essential gene bracketed by DNA sequences found upstream and downstream of the target chromosomal gene. Exemplary target genes include, but are not limited to, rpoA, rpoB, gyrA, gyrB, fabG, fabI, and fusA. Modulation of the expression of such target genes can be performed, as noted above, to provide a host strain in which the gene product of the target gene is rate-limiting for growth and/or virulence and which serves as an indicator strain for the detection of compounds active against the product encoded by the target gene.

6.3. Construction, Identification, and Use of Modified Tetracycline Repressors Exhibiting a Reverse Phenotype in Gram Negative Bacteria

Construction, Identification, and Use of revTetR Repressors in Pseudomonas aeruginosa

A pool of mutated Tet repressors is created as in Example 6.1 and cloned into an expression vector comprising a promoter active in Pseudomonas aeruginosa, such as the T7 promoter of E. coli bacteriophage T7 or the recA promoter. Alternatively, each of the nucleotide sequences of SEQ ID NOS. 1, 3, 5, 7, 9, 11, 13, 15, 17, 19, 21, 23, 25, 27, 29, and 265-458, is operatively associated with a promoter active in Pseudomonas aeruginosa, such as the T7 and recA promoter, and the recombinant gene expressing a revTetR protein so produced is introduced into Pseudomonas aeruginosa to confirm the revTetR phenotype in this host. In preferred embodiments, the promoter active in Pseudomonas aeruginosa is regulated; for example, the level of transcription from a recA-promoter can be induced by exposing the host cell to nalidixic acid.

The revTetR phenotype is determined, in certain embodiments, by analyzing the expression of a reporter gene selected from the group consisting of lacZ, GFP, and luxA, that is under the control of a promoter active in Pseudomonas aeruginosa, which promoter has been engineered to comprise at least one tetracycline operator sequence. Accordingly, expression of such indicator genes is repressed by a revTetR repressor in the presence of subinhibitory levels of tetracycline, anhydrotetracycline or other suitable tetracycline analogue. In alternative instances, a direct selection, rather than a screen, is established to allow the isolation of the revTetR mutants in Pseudomonas aeruginosa using the strategy described above in Section 6.1. For example, an antibiotic resistance gene, such as a gene encoding kanamycin resistance, can be place under the control of a negative-regulatory element, such as a repressor protein. The repressor protein, in turn is operatively associated with one or more tet operators such that expression of the repressor results in sensitivity of the host cell to, e.g., kanamycin, in the presence of a wild-type TetR protein in the absence of sub-inhibitory levels of tetracycline, anhydrotetracycline, or other suitable tetracycline analog. In this embodiment, revTetR mutants are selected as kanamycin-resistant in the absence of tetracycline, anhydrotetracycline, or other suitable tetracycline analog, and the revTetR phenotype confirmed by demonstrating kanamycin-sensitivity in the presence of sub-inhibitory levels of tetracycline, anhydrotetracycline, or other suitable tetracycline analog.

Exemplary promoters, which are active in the gram negative organism, Pseudomonas aeruginosa, are modified so as to be placed under tetR regulation; examples of such promoters include but are not limited to the T7, mini-T7, anaerobically-inducible arcDABC operon promoter, the lac-repressor -regulated trc promoter, and the nalidixic-acid-inducible recA promoter (see Hoang et al., 2000, Plasmid, 43: 59-72); each of these promoters can be engineered to include one or more tet operators to provide a tetracycline-regulated promoter that can be operatively associated with a target or indicator gene of interest.

In certain embodiments, either one or both of the gene encoding the revTetR repressor and the gene encoding the tetracycline-regulated indicator gene are integrated into the Pseudomonas aeruginosa chromosome via homologous recombination or by using integration-proficient plasmids such as, but not limited to, mini-CTX1 and mini-CTX2 (Hoang et al. 2000 Plasmid 45: 59-72. In certain other embodiments, either or both of the gene encoding the revTetR repressor and the gene encoding the tetracycline regulated indicator gene are maintained episomally. Both may be episomal and carried on different replicons where the plasmids are compatible and different selectable markers are used. Such recombinant nucleic acids are introduced into Pseudomonas aeruginosa or other gram-negative prokaryotic organisms by electroporation, using methods known to those of ordinary skill in the art. Suitable selective markers useful for selection in Pseudomonas aeruginosa include, but are not limited to, tetracycline resistance, ampicillin resistance, and streptomycin resistance. Appropriate Pseudomonas aeruginosa host strains include, but are not limited to, ADD1976 and PAO1. One example of a suitable growth medium is LB medium, which includes, per liter, 10 g tryptone, 5 g yeast extract, and 5 g sodium chloride; this medium is generally supplemented with a carbon source, such as glucose or glycerol (e.g. to a level of 0.2% ) as desired.

Where the reporter gene expresses β-galactosidase (lacZ), revTetR-encoding genes may be identified using the screen disclosed in EXAMPLE 6.1. Recombinant DNA can be isolated from the identified organisms, and the sequences encoding the revTetR repressors can be determined by methods known in the art.

Suitable plasmid vectors useful for molecular cloning in Pseudomonas aeruginosa include Pseudomonas—E. coli shuttle vectors such as but not limited to pUCP19 derivatives such as pUCPKS, and pUCPSK (Watson et al. Gene 172: 163-64), IncQ-compatiblity plasmids comprising the arcDABC operon promoter (Winteler et al. 1996, Appl. Environ. Microbiol. 62: 3391-98), vectors comprising the nalidixic acid inducible recA promoter (Rangwala et al. 1991, Biotechnology, 2: 477-79), and plasmids pUM505 and pSUP104, which encode chromate resistance (Cervantes et al. 1990, J. Bacteriol. 172: 287-91).

Tet-regulated expression of potential target genes/essential genes in Pseudomonas aeruginosa is achieved in one non-limiting example, by allele replacement based upon homologous recombination between non-replicating episomal DNA carrying a tet-operator-regulated essential gene bracketed by DNA sequences found upstream and downstream of the target chromosomal gene. Exemplary target genes include, but are not limited to, rpoA, rpoB, gyrA, gyrB, fabG, fabI, and fusA. Modulation of the expression of such target genes can be performed, as noted above, to provide a host strain in which the gene product of the target gene is rate-limiting for growth and/or virulence and which serves as an indicator strain for the detection of compounds active against the product encoded by the target gene.

6.4 Construction of RevTetR Proteins Using Oligonucleotide directed Randomization Mutagenesis

Random mutations were introduced into three distinct regions of the DNA sequence encoding TetR. Mutagenesis within each region of the TetR coding region was carried out according to the “three-primer” method of Landt et al. (Landt et al. (1990) “A General Method for Rapid Site-directed Mutagenesis Using the Polymerase Chain Reaction,” Gene 96: 125-128, which is hereby incorporated by reference in its entirety). The three regions subjected to this site-directed mutagenic procedure were the coding regions for amino acids 14-25, for amino acids 48-63, and for amino acids 93-102. In each instance three oligonucleotides were prepared. Two of the oligonucleotides were upstream and downstream PCR primers for the region to be mutagenized. The third, mutagenic, “partially randomized” primer was synthesized so as to contain, at each nucleotide position within the sequence for the region to be mutagenized, approximately 85% wild-type base with the remainder distributed among the other three, non-wild type bases for that position. For example, the partially randomized primer used for mutagenesis of the coding region for amino acids 48-63 of SEQ ID NO: 32 had the following nucleotide sequence (SEQ ID NO: 459):

5′-ATAATCATGATGACGCGCCAAGATCTCCACCGCCAGCGCATCCAGTAGGGCCCGCTTATTTTTTAC-3′,

wherein each underlined base was present in approximately 85% of the oligonucleotides, while the remaining approximately 15% of the oligonucleotides contained one of the other three, non-wild type bases at that position. Similar mutagenic, partially randomized oligonucleotides were prepared for mutagenesis of the coding regions for amino acids 14-25 and 93-102. It was predicted that, according to a binomial distribution, each oligonucleotide would contain three to four mutations. PCR amplification reactions were carried out using the three indicated oligonucleotides (upstream, downstream, and mutagenic partially randomized oligonucleotide primers) according to the method of Landt using pWH1411 plasmid DNA as template. Accordingly, three PCR products comprising mutagenized sequences were obtained corresponding, respectively, to the coding regions for amino acids 14-25, 48-63, and 93-102. Each pool was separately cloned into the corresponding region of the TetR coding sequence. In addition, each of the three possible pairs (coding regions for amino acids 14-25 and 48-63; coding regions for amino acids 14-25 and 93-102; and coding regions for amino acids 48-63 and 93-102) of PCR products were also inserted, using genetic engineering methodology, into the coding region of the TetR protein. All six pools of mutagenized TetR sequences were screened for TetR variants having a reverse phenotype. Transformed strains are analyzed using the materials and methods disclosed in Section 6.1, above. Isolates carrying mutant TetR proteins exhibiting a reverse phenotype that were obtained using this procedure include those designated TetRevAtc4-1 to TetRevAF6/5 of Tables 1, 2, and 6. More specifically, the clone designation, SEQ ID NO:, and identified amino acid substitution(s) are provided in Table 1; the clone designation, SEQ ID NO:, and identified nucleotide substitution(s) are provided in Table 2; and the clone designation and activity of non-repressed and repressed levels of β-galactosidase activity (i.e. in the absence and in the presence of anhydrotetracycline) are shown in Table 6, below.

6.5 Construction of RevTetR Proteins Using Oligonucleotide directed Randomization Mutagenesis of the Coding Sequence for Amino Acid 96 and Amino Acid 96 and 99

Site specific mutagenesis was also carried out that was directed toward either the codon for amino acid 96 alone or for codons 96 and 99 simultaneously. Again, the site-directed mutagenesis was carried out according to the “three-primer” method of Landt. However, in this instance, the mutagenic oligonucleotide was randomized only with respect to the particular codon or pair of codons to be mutagenized; in each case the wild type sequence was replace with the triplet NNS (where N is any nucleotide, i.e. A, T, G, or C, and S is the single-letter code indicating that the nucleotide at this position is either C or G). Plasmid DNA (pWH1411) was used as the template for the PCR amplification reactions. Transformed strains are analyzed using the materials and methods disclosed in Section 6.1, above. Isolates carrying mutant TetR proteins exhibiting a reverse phenotype that were obtained using this procedure include those designated TetRev 96/99-1 to TetRev 96/99-P of Tables 1, 2, and 6. More specifically, the clone designation, SEQ ID NO., and identified amino acid substitution(s) are provided in Table 1; the clone designation, SEQ ID NO., and identified nucleotide substitution(s) are provided in Table 2; and the clone designation and activity of non-repressed and repressed levels of β-galactosidase activity (i.e. in the absence and in the presence of anhydrotetracycline) are shown in Table 6:

TABLE 6
β-Galactosidase Activity of RevTetR Isolates
Cultured in the Presence and the
Absence of Anhydrotetracycline (ATC)

| | Cultured | Standard | Cultured | Standard |
|---|---|---|---|---|
| | Without | Deviation | With | Deviation |
| RevTetR Isolate | ATC | (without ATC) | ATC | (with ATC) |
| TetRrevAtc4-1 | 100.076 | 3.2043 | 6.9011 | 3.4749 |
| TetRrevAtc4-10 | 69.401 | 3.557 | 11.275 | 0.576 |
| TetRrevAtc4-11 | 103.132 | 4.935 | 17.479 | 1.119 |
| TetRrevAtc4-13 | 98.8175 | 10.634 | 8.1397 | 0.294 |
| TetRrevAtc4-14 | 62.985 | 1.189 | 25.025 | 0.754 |
| TetRrevAtc4-16 | 104.174 | 1.8764 | 16.914 | 0.2459 |
| TetRrevAtc4-2 | 3.14294 | 1.1491 | 0.9516 | 0.0843 |
| TetRrevAtc4-20 | 78.478 | 3.34 | 8.048 | 0.754 |
| TetRrevAtc4-21 | 104.757 | 5.262 | 16.831 | 1.603 |
| TetRrevAtc4-22 | 80.2859 | 1.7426 | 3.7584 | 0.5517 |
| TetRrevAtc4-23 | 105.95 | 8.641 | 21.606 | 1.904 |
| TetRrevAtc4-24 | 61.649 | 4.011 | 13.63 | 0.271 |
| TetRrevAtc4-25 | 98.629 | 4.534 | 12.116 | 0.833 |
| TetRrevAtc4-28 | 85.141 | 1.931 | 4.217 | 0.251 |
| TetRrevAtc4-31 | 65.039 | 10.041 | 2.181 | 0.163 |
| TetRrevAtc4-4 | 70.5665 | 2.253 | 2.5363 | 0.2812 |
| TetRrevAtc4-40 | 56.743 | 4.67 | 14.428 | 2.894 |
| TetRrevAtc4-43 | 99.61 | 10.258 | 13.878 | 0.611 |
| TetRrevAtc4-47 | 83.987 | 8.117 | 13.969 | 1.868 |
| TetRrevAtc4-48 | 110.179 | 1.696 | 16.146 | 1.515 |
| TetRrevAtc4-5 | 98.272 | 3.25 | 12.552 | 0.342 |
| TetRrevAtc4-52 | 105.367 | 0.999 | 5.575 | 0.604 |
| TetRrevAtc4-53 | 87.055 | 0.965 | 6.945 | 1.407 |
| TetRrevAtc4-6 | 108.478 | 7.148 | 23.873 | 0.573 |
| TetRrevAtc4-61 | 88.785 | 2.032 | 23.831 | 3.674 |
| TetRrevAtc4-67 | 61.815 | 6.352 | 12.616 | 1.458 |
| TetRrevAtc4-7 | 84.416 | 4.208 | 27.649 | 1.619 |
| TetRrevAtc4-70 | 16.119 | 0.847 | 11.125 | 0.834 |
| TetRrevAtc4-71 | 70.197 | 1.416 | 7.372 | 4.434 |
| TetRrevAtc4-9 | 97.1039 | 2.5704 | 1.8695 | 0.0705 |
| TetRrevAtc4-9b | 107.982 | 4.4152 | 1.0594 | 0.1013 |
| TetRrevDox4-1 | 36.03 | 1.518 | 5.627 | 0.316 |
| TetRrevDox4-2 | 58.776 | 4.54 | 9.547 | 0.705 |
| TetRrev04-1 | 102.092 | 2.934 | 44.145 | 1.246 |
| TetRrev04-4 | 99.655 | 3.09 | 19.878 | 4.306 |
| TetRrev6-13 | 15.7835 | 0.859 | 2.5043 | 0.1865 |
| TetRrev6-17 | 56.5081 | 1.4194 | 12.5 | 1.4 |
| TetRrev6-2 | 95.487 | 3.355 | 18.3 | 6.815 |
| TetRrev6-23 | 101 | 4.9 | 49 | 2 |
| TetRrev6-25 | 42.8584 | 1.5726 | 4.136 | 0.256 |
| TetRrev6-26 | 53.6535 | 4.9827 | 6.9688 | 0.3698 |
| TetRrev6-27 | 108.105 | 10.073 | 44.43 | 16.272 |
| TetRrev6-3 | 23.0695 | 4.7672 | 1.3657 | 0.3418 |
| TetRrev6-31 | 55.658 | 0.825 | 35.26 | 9.053 |
| TetRrev6-32 | 79.3731 | 3.2936 | 3.789 | 0.165 |
| TetRrev6-33 | 108.964 | 5.645 | 2.984 | 0.213 |
| TetRrev6-34 | 85.725 | 2.248 | 40.254 | 3.489 |
| TetRrev6-35 | 74.8797 | 8.3714 | 7.0255 | 0.462 |
| TetRrev6-37-1 | 55.277 | 6.786 | 20.04 | 2.14 |
| TetRrev6-38 | 96.194 | 2.689 | 46.91 | 9.409 |
| TetRrev6-50 | 60.464 | 2.328 | 2.469 | 1.081 |
| TetRrev6-51 | 93.974 | 3.312 | 4.499 | 0.845 |
| TetRrev6-53 | 107.107 | 3.239 | 27.918 | 2.095 |
| TetRrev6-54 | 6.393 | 0.923 | 1.268 | 0.019 |
| TetRrev4/6-3 | 69.291 | 9.307 | 0.878 | 0.096 |
| TetRrev4/6-4 | 96.491 | 1.081 | 1.639 | 0.203 |
| TetRrev4/6-5 | 97.92 | 3.486 | 5.7 | 0.321 |
| TetRrev4/6-6 | 67.879 | 5.678 | 3.197 | 0.161 |
| TetRrev4/6-7 | 86.036 | 2.657 | 2.151 | 0.119 |
| TetRrev4/6-10 | 97.964 | 1.525 | 6.149 | 0.293 |
| TetRrev4/6-15 | 66.054 | 2.358 | 7.647 | 0.266 |
| TetRrev4/6-17 | 54.403 | 6.124 | 2.601 | 0.149 |
| TetRrev4/6-24 | 95.596 | 0.889 | 2.428 | 0.067 |
| TetRrev4/6-25 | 102.926 | 3.614 | 3.433 | 0.044 |
| TetRrev4/6-27 | 108 | 10 | 49 | 4.8 |
| TetRrev1/34 | 79.3141 | 3.7375 | 0.9226 | 0.011 |
| TetRrev3/38 | 97.043 | 1.9557 | 1.8287 | 0.3367 |
| TetRrev19/48 | 79.9581 | 3.4181 | 1.1143 | 0.0463 |
| TetRrev22/5 | 92.312 | 2.3888 | 0.9755 | 0.0213 |
| TetRrev25/43 | 99.1241 | 7.9256 | 1.7535 | 0.083 |
| TetRrev28/8 | 98.6289 | 4.9401 | 4.4123 | 0.3144 |
| TetRrev28/16 | 96.3269 | 1.2921 | 0.9453 | 0.0233 |
| TetRrev28/23 | 92.2055 | 2.5032 | 2.9867 | 0.0966 |
| TetRrev28/26 | 89.3918 | 1.8774 | 3.8006 | 0.0692 |
| TetRrev28/27 | 95.0152 | 4.3584 | 1.6907 | 0.0693 |
| TetRrev28/30 | 55.1783 | 5.2783 | 3.7384 | 0.0407 |
| TetRrev28/31 | 99.8749 | 1.4435 | 1.1121 | 0.0621 |
| TetRrev28/36 | 65.5335 | 2.7194 | 1.0579 | 0.0548 |
| TetRrev28/40 | 91 .691 | 2.3601 | 3.4834 | 0.8788 |
| TetRrev28/41 | 15.4643 | 1.2494 | 5.6333 | 0.1529 |
| TetRrev28/46 | 95.9627 | 2.011 | 1.1482 | 0.0337 |
| TetRrev28/48 | 99.3107 | 2.4637 | 1.1587 | 0.051 |
| TetRrev28/49 | 62.5137 | 5.9944 | 8.2716 | 0.2795 |
| TetRrev29/9 | 74.4357 | 3.6638 | 11.772 | 0.7402 |
| TetRrev29/17 | 44.6409 | 5.7567 | 1.7738 | 0.0666 |
| TetRrev29/24 | 85.5708 | 4.1606 | 7.9819 | 1.0449 |
| TetRrev29/25 | 92.9327 | 3.2324 | 3.112 | 0.4435 |
| TetRrev29/27 | 82.7579 | 3.671 | 9.453 | 0.4703 |
| TetRrev29/35 | 58.0891 | 2.6235 | 2.1051 | 0.1521 |
| TetRrev29/42 | 96.187 | 1.9875 | 11.861 | 4.2925 |
| TetRrev29/52 | 48.4964 | 2.7558 | 3.3035 | 0.2923 |
| TetRrevAD1/2 | 84.85 | 2.0606 | 1.6137 | 0.7902 |
| TetRrevAD1/6 | 85.6043 | 0.8583 | 2.5079 | 0.1054 |
| TetRrevAF1/7 | 72.199 | 0.6256 | 2.2753 | 0.0949 |
| TetRrevAF1/8 | 42.088 | 1.3939 | 1.1643 | 0.0385 |
| TetRrevAF1/11 | 96.2362 | 4.9178 | 1.1578 | 0.0062 |
| TetRrevAF2/5 | 97.8373 | 0.9757 | 2.4067 | 0.1718 |
| TetRrevAD2/4 | 32.2603 | 2.2984 | 1.0068 | 0.0326 |
| TetRrevAD2/6 | 89.5374 | 4.1857 | 3.8811 | 0.0424 |
| TetRrevAD2/12 | 73.5981 | 1.6391 | 1.2806 | 0.0064 |
| TetRrevAD2/13 | 71.1255 | 1.2898 | 1.3355 | 0.0277 |
| TetRrevAD2/2 | 94.968 | 0.902 | 5.2312 | 0.3179 |
| TetRrevAF1/3 | 59.773 | 1.257 | 1.2536 | 0.0174 |
| TetRrevAF1/4 | 81.2613 | 2.204 | 13.459 | 0.509 |
| TetRrevAF1/5 | 102.015 | 8.58 | 1.1988 | 0.0833 |
| TetRrevAD3/2 | 60.3284 | 4.1931 | 3.5891 | 0.1647 |
| TetRrevAF2/7 | 70.3163 | 4.5063 | 2.8977 | 0.2432 |
| TetRrevAF6/12 | 82.6734 | 10.966 | 1.3259 | 0.0137 |
| TetRrevAF7/1 | 89.3536 | 6.1618 | 1.3792 | 0.0548 |
| TetRrevAF7/2 | 58.8637 | 6.1831 | 3.7321 | 0.3709 |
| TetRrevAD3/5 | 89.8129 | 4.6758 | 1.1481 | 0.0472 |
| TetRrevAD3/6 | 93.0552 | 7.6646 | 1.6494 | 0.0369 |
| TetRrevAD3/7 | 73.4537 | 5.3256 | 2.4531 | 0.3358 |
| TetRrevAD3/8 | 65.0174 | 2.4169 | 1.9046 | 0.2399 |
| TetRrevAF2/14 | 90.5688 | 1.1264 | 1.239 | 0.0063 |
| TetRrevAF2/15 | 82.5755 | 2.0008 | 2.828 | 0.1574 |
| TetRrevAF2/16 | 90.6438 | 3.6313 | 2.622 | 0.1294 |
| TetRrevAF3/5 | 101.824 | 3.9 | 4.696 | 1.4121 |
| TetRrevAD3/9 | 60.2932 | 2.0912 | 4.2427 | 0.2096 |
| TetRrevAD3/10 | 89.1669 | 4.4232 | 3.5004 | 0.0747 |
| TetRrevAF3/6 | 77.2272 | 1.5973 | 1.0546 | 0.0305 |
| TetRrevAF3/8 | 95.5476 | 4.2398 | 2.7257 | 0.208 |
| TetRrevAF4/4 | 94.4528 | 5.0745 | 3.8273 | 0.4438 |
| TetRrevAF4/5 | 105.33 | 5.7659 | 0.9927 | 0.0229 |
| TetRrevAF4/6 | 100.702 | 5.712 | 3.7257 | 0.2822 |
| TetRrevAF4/9 | 110.616 | 4.8699 | 1.3761 | 0.0546 |
| TetRrevAD2/5 | 50.466 | 0.7082 | 1.4141 | 0.0554 |
| TetRrevAD2/8 | 73.8584 | 3.1264 | 1.8468 | 0.1056 |
| TetRrevAD2/1 | 34.2242 | 1.7633 | 1.17 | 0.0786 |
| TetRrevAF4/13 | 98.8886 | 3.559 | 2.5827 | 0.0302 |
| TetRrevAF5/1 | 100.053 | 6.8865 | 1.072 | 0.0294 |
| TetRrevAF5/3 | 58.5651 | 3.8923 | 1.8337 | 0.0109 |
| TetRrevAF5/5 | 100.546 | 6.1541 | 1.6948 | 0.0753 |
| TetRrevAF5/13 | 105.942 | 7.7376 | 4.4642 | 0.3006 |
| TetRrevAF6/1 | 72.8186 | 3.8996 | 3.7966 | 0.0857 |
| TetRrevAF6/5 | 102.848 | 2.9009 | 2.8572 | 0.2495 |
| TetRrev96/99-1 | 58.4545 | 2.5556 | 25.484 | 1.5401 |
| TetRrev96/99-2 | 94.5832 | 8.2982 | 31.604 | 1.3004 |
| TetRrev96/99-3 | 103.749 | 3.0429 | 31.808 | 0.5679 |
| TetRrev96/99-4 | 109.291 | 3.0636 | 32.997 | 0.5983 |
| TetRrev96/99-5 | 57.8391 | 3.2552 | 17.087 | 1.2772 |
| TetRrev96/99-6 | 103.602 | 2.0728 | 28.364 | 0.9685 |
| TetRrev96/99-7 | 55.0127 | 5.0167 | 13.224 | 0.4851 |
| TetRrev96/99-8 | 103.657 | 2.4813 | 24.309 | 0.6575 |
| TetRrev96/99-9 | 71.6829 | 1.566 | 14.528 | 0.7889 |
| TetRrev96/99-10 | 106.137 | 4.517 | 20.797 | 2.1892 |
| TetRrev96/99-11 | 97.6643 | 2.1647 | 18.656 | 0.7293 |
| TetRrev96/99-12 | 102.406 | 3.3595 | 18.841 | 0.6756 |
| TetRrev96/99-13 | 103.963 | 2.0753 | 16.95 | 0.8005 |
| TetRrev96/99-14 | 110.999 | 1.9805 | 14.693 | 0.3837 |
| TetRrev96/99-15 | 110.413 | 5.3214 | 14.218 | 0.9044 |
| TetRrev96/99-16 | 93.7512 | 3.5679 | 9.8301 | 0.6439 |
| TetRrev96/99-17 | 92.9198 | 1.8727 | 8.4558 | 0.0755 |
| TetRrev96/99-18 | 85.7142 | 4.2359 | 7.5138 | 1.126 |
| TetRrev96/99-19 | 95.188 | 1.0819 | 7.4009 | 0.1084 |
| TetRrev96/99-20 | 81.8583 | 2.1543 | 3.0074 | 0.1654 |
| TetRrev96P | 24.1109 | 0.9607 | 4.1882 | 2 |

The present invention is not to be limited by the scope of the specific embodiments described herein. Indeed, various modifications of the invention in addition to those described herein will become apparent to those of skill in the art from the foregoing description and accompanying figures. Such modifications are intended to fall within the scope of the appended claims.

Various publications are cited herein, the disclosures of which are hereby incorporated by reference in their entireties.

What is claimed is:

1. An isolated nucleic acid that comprises a nucleotide sequence selected from the group consisting of SEQ ID NO: 1, 3, 5, 7, 9, 11, 13, 15, 17, 19, 21, 23, 25, 27, 29, or 265-458 or a nucleotide sequence that encodes an amino acid sequence selected from the group consisting of SEQ ID NO: 2, 4, 6, 8, 10, 12, 14, 16, 18, 20, 22, 24, 26, 28, 30, or 71-264.

2. An isolated nucleic acid that encodes a modified tetracycline repressor that:
(i) binds to a tetracycline operator sequence in a prokaryotic organism with a greater affinity in the presence of tetracycline or a tetracycline analog than in the absence of tetracycline or a tetracycline analog;
(ii) comprises at least one amino acid substitution that corresponds to an amino acid substitution present in an amino acid sequence selected from the group consisting of SEQ ID NO: 2, 4, 6, 8, 10, 12, 14, 16, 18, 20, 22, 24, 26, 28, 30 and 71-264; and wherein said nucleic acid
(iii) hybridizes under high stringency over substantially the entire length to a nucleic acid probe consisting of SEQ ID NO: 31, or
(iv) has at least 60 % nucleotide sequence identity to SEQ ID NO: 31.

3. An isolated nucleic acid that encodes a modified tetracycline repressor that
(i) binds to a tetracycline operator sequence in a prokaryotic organism with a greater affinity in the presence of tetracycline or a tetracycline analog than in the absence of tetracycline or a tetracycline analog; and
(ii) comprises at least one amino acid substitution that corresponds to an amino acid substitutions present in an amino acid sequence selected from the group consisting of SEQ ID NO: 2, 4, 6, 8, 10, 12, 14, 16, 18, 20, 22, 24, 26, 28, 30 and 71-264 as compared to an unmodified tetracycline repressor of any one of tet(A), tet(B), tet(C), tet(D), Tet(E), tet(G), tet(H), tet(J), or tet(Z) family.

4. The isolated nucleic acid of claim 3, wherein the unmodified tetracycline repressor comprises an amino acid sequence selected from the group consisting of SEQ ID NO: 34, 36, 38, 40, 42, 44, 46, 48 and 50.

5. The isolated nucleic acid of claim 3, wherein the nucleic acid
(i) hybridizes under high stringency over substantially the entire length to a nucleic acid probe consisting of a nucleotide sequence selected from the group consisting of SEQ ID NO: 33, 35, 37, 39, 41, 43, 45, 47, and 49, or
(ii) has at least 80% nucleotide sequence identity to a nucleic acid selected from the group consisting of SEQ ID NO: 33, 35, 37, 39, 41, 43, 45, 47, and 49; or
(iii) encodes a polypeptide that has at least 80% amino acid sequence identity to a peptide sequence selected from the group consisting of SEQ ID NO: 34, 36, 38, 40, 42, 44, 46, 48, and 50.

6. The isolated nucleic acid of claim 2 or 3, wherein the prokaryotic organism is a bacterium.

7. The isolated nucleic acid of claim 6, wherein the bacterium is a gram-positive bacterium.

8. The isolated nucleic acid of claim 6, wherein the bacterium is a gram-negative bacterium.

9. The isolated nucleic acid of claim 6, wherein the prokaryotic organism is an archaeabacterium.

10. An isolated nucleic acid, comprising a fragment of one of SEQ ID NO: 1, 3, 5, 7, 9, 11, 13, 15, 17, 19, 21, 23, 25, 27, 29, and 265-458, said fragment selected from the group consisting of fragments comprising at least 10, at least 20, at least 25, at least 30, at least 50 and at least 100 consecutive nucleotides of one of SEQ ID NO: 1, 3, 5, 7, 9, 11, 13, 15, 17, 19, 21, 23, 25, 27, 29, and 265-458, wherein said fragment encodes an amino substitution selected from the group consisting of I 59 N, A 70 V, A 71 V, L 91 Q, D 95 E, D 95 G, G 96 R, G 96 E, K 98 R, V 99 E, H 100 A, L 101 H, T 103 S, Y 110 F, E 114 V, L 127 R, D 157 N, R 158 C, P 159 L, A 160 V, D 178 V, H 188 Q, S 192 G, I 194 V, G 196 W, Q 200 H, and L 205 S, as compared to the amino acid sequence of SEQ ID NO: 32.

11. The isolated nucleic acid of claim 1, 2, or 3, further comprising a promoter operatively associated with the nucleotide sequence encoding the modified tetracycline repressor.

12. A vector comprising an isolated nucleic acid of claim 1, 2, or 3.

13. A prokaryotic organism comprising an isolated nucleic acid of claim 1, 2, or 3.

14. The prokaryotic organism of claim 13, wherein the prokaryotic organism is selected from the group consisting of Bacillus anthracis, Bacteriodes fragilis, Bordetella pertussis, Burkholderia cepacia, Camplyobacter jejuni, Chlamydia pneumoniae, Chlamydia trachomatus, Clostridium botulinum, Clostridum tetani, Clostridium perfringens, Clostridium difficile, Corynebacterium diptheriae, Enterobacter clocae, Enterococcus faecalis, Escherichia coli, Haemophilus influenzae, Helicobacter pylori, Klebsiella pneumoniae, Listeria monocytogenes, Moraxella catarrhalis, Mycobacterium leprae, Mycobacterium tuberculosis, Neisseria gonorrhoeae, Nesseria meningitidis, Nocardia asteroides, Proteus vulgaris, Pseudomonas aeruginosa, Salmonella typhi, Salmonella typhimurium, Shigella boydii, Shigella dysenteriae, Shigella flexneri, Shigella sonnei, Staphylococcus aureus, Staphylococcus epidermidis, Streptococcus mutans, Streptococcus pneumoniae, Treptonema pallidum, Vibrio cholerae, Vibrio parahemolyticus, and Yersina pestis.

15. A method for preparing a modified tetracycline repressor that binds to a tetracycline operator sequence in a prokaryotic organism with a greater affinity in the presence of tetracycline or a tetracycline analog than in the absence of tetracycline or a tetracycline analog, comprising:
introducing into a prokaryotic organism an expression vector comprising the nucleic acid of claim 1, 2, or 3 in operative association with a promoter; and
expressing the modified tetracycline repressor protein in the prokaryotic organism.

16. An isolated modified tetracycline repressor protein comprising an amino acid sequence selected from the group consisting of SEQ ID NO: 2, 4, 6, 8, 10, 12, 14, 16, 18, 20, 22, 24, 26, 28, 30, and 71-264.

17. An isolated modified tetracycline repressor protein comprising an amino acid sequence encoded by the isolated nucleic acid of claim 1, 2, or 3.

18. A method for identifying a modified tetracycline repressor that binds to a tetracycline operator sequence in a prokaryotic organism with a greater affinity in the presence of tetracycline or a tetracycline analog than in the absence of tetracycline or a tetracycline analog, comprising:
introducing into the prokaryotic organism a nucleic acid that comprises a reporter gene operatively linked to a promoter regulated by a tetracycline operator, and an expressible nucleic acid encoding a modified tetracycline repressor containing at least one amino acid substitution relative to a wild type tetracycline repressor, wherein the wild type tetracycline repressor binds the tetracycline operator with a greater affinity in the absence of tetracycline or the tetracycline analog than in the presence of tetracycline or the tetracycline analog;
culturing the prokaryotic organism in the presence or absence of tetracycline or the tetracycline analog, and under conditions such that the modified tetracycline repressor is expressed; and
identifying the prokaryotic organism that expresses the reporter gene at a higher level in the absence than in the presence of the tetracycline or the tetracycline analog.

19. The method of claim 18, wherein the nucleotide sequence encoding the modified tetracycline repressor hybridizes under stringent conditions to a nucleic acid probe consisting of the nucleotide sequence of SEQ ID NO: 31.

20. A method for identifying a gene essential for proliferation or pathogenicity of a prokaryotic organism, comprising:
culturing a prokaryotic organism, comprising a first expressible nucleic acid encoding a putative essential gene under the control of at least one tet operator and a second expressible nucleic acid encoding a modified tetracycline repressor that binds to a tetracycline operator sequence in a prokaryotic organism with a greater affinity in the presence of tetracycline or a tetracycline analog than in the absence of tetracycline or the tetracycline analog, under conditions such that the modified tetracycline repressor is expressed and in the presence of a sub-inhibitory concentration of tetracycline or a tetracycline analog sufficient to repress expression of the putative essential gene; and
determining the viability or pathogenicity of the organism, whereby an decrease in or lack of proliferation or pathogenicity in the presence of tetracycline or the tetracycline analog indicates that the gene is essential.

21. The method of claim 20, wherein the second expressible nucleic acid comprises the nucleic acid of claim 1, 2, or 3.

22. A method for identifying a compound that inhibits an essential gene or gene product of a prokaryotic organism, comprising:
culturing a prokaryotic organism, comprising a first expressible nucleic acid encoding the essential gene under the control of at least one tet operator and a second expressible nucleic acid encoding a modified tetracycline repressor that binds to a tetracycline operator sequence in a prokaryotic organism with a greater affinity in the presence of tetracycline or a tetracycline analog than in the absence of tetracycline or a tetracycline analog, under conditions such that the modified tetracycline repressor is expressed and in the presence of a sub-inhibitory concentration of tetracycline or a tetracycline analog sufficient to repress expression of the essential gene;
contacting the prokaryotic organism with a test compound; and
determining the effect of the test compound compared to control cells not cultured in tetracycline or tetracycline analog.

23. A method for identifying a compound that inhibits an essential gene
or gene product of a prokaryotic organism, comprising:
culturing a prokaryotic organism, comprising a first expressible nucleic acid encoding the essential gene under the control of at least one tet operator and a second expressible nucleic acid encoding a modified tetracycline repressor that binds to a tetracycline operator sequence in a prokaryotic organism with a greater affinity in the presence of tetracycline or a tetracycline analog than in the absence of tetracycline or a tetracycline analog, under conditions such that the modified tetracycline repressor is expressed and in the presence of a sub-inhibitory concentration of tetracycline or a tetracycline analog sufficient to repress expression of the essential gene;
contacting the prokaryotic organism with a test compound; and
determining the effect of the test compound compared to control cells cultured under the same conditions, wherein the control cells comprise said second expressible nucleic acid.

24. The method of claim 22, wherein the second expressible nucleic acid comprises the nucleic acid of claim 1, 2, or 3.

25. A method for identifying a compound that modulates the binding affinity of a modified tetracycline repressor to a tetracycline operator sequence in a prokaryotic organism, wherein the modified tetracycline repressor binds the tetracycline operator with a greater affinity in the presence of tetracycline or a tetracycline analog than in the absence of tetracycline or a tetracycline analog, comprising:
culturing a prokaryotic organism, comprising a nucleic acid comprising a reporter gene operatively linked to a promoter regulated by a tetracycline operator and said organism further comprising an expression vector comprising a nucleotide sequence encoding the modified tetracycline repressor, in the presence or absence of the compound under conditions such that the modified tetracycline repressor is expressed; and
identifying the compound that modulates expression of the reporter gene product.

26. The method of claim 25, wherein the modified tetracycline repressor is encoded by a nucleic acid of claim 1, 2, or 3.

27. A method for identifying a compound that inhibits an essential gene product of a prokaryotic organism, comprising:
infecting a first and a second animal with a prokaryotic organism comprising a nucleic acid comprising a nucleotide sequence encoding the essential gene under the control of at least one tet operator, said organism further comprising an expressible nucleic acid encoding a modified tetracycline repressor that binds to a tetracycline operator sequence in a prokaryotic organism with a greater affinity in the presence of tetracycline or a tetracycline analog than in the absence of tetracycline or a tetracycline analog, said first animal being provided with tetracycline or a tetracycline analog at a concentration sufficient to substantially repress expression of the essential gene in the prokaryotic organism;
contacting said first and second animals with a test compound; and
determining the effect of the test compound on said first and said second animals.

28. The method of claim 27, wherein the prokaryotic organism is selected from the group consisting of Bacillus anthracis, Bacteriodes fragilis, Bordetella pertussis, Burkholderia cepacia, Camplyobacter jejuni, Chlamydia pneumoniae, Chlamydia trachomatus, Clostridium botulinum, Clostridum tetani, Clostridium perfringens, Clostridium difficile, Corynebacterium diptheriae, Enterobacter clocae, Enterococcus faecalis, Escherichia coli, Haemophilus influenzae, Helicobacter pylori, Klebsiella pneumoniae, Listeria monocytogenes, Moraxella catarrhalis, Mycobacterium leprae, Mycobacterium tuberculosis, Neisseria gonorrhoeae, Nesseria meningitidis, Nocardia asteroides, Proteus vulgaris, Pseudomonas aeruginosa, Salmonella typhi, Salmonella typhimurium, Shigella boydii, Shigella dysenteriae, Shigella flexneri, Shigella sonnet, Staphylococcus aureus, Staphylococcus epidermidis, Streptococcus mutans, Streptococcus pneumoniae, Treptonema pallidum, Vibrio cholerae, Vibrio parahemolyticus, and Yersina pestis.

29. A method for correlating an expressed protein detected in proteomics analyses with a gene encoding the expressed protein, the method comprising the steps of:
(a) developing a first protein expression profile for a control prokaryotic organism, wherein the control organism comprises a target gene;
(b) providing a derivative of the control prokaryotic organism wherein said derivative comprises an expressible nucleic acid encoding a revTetR according to claim 1, 2, or 3, and wherein the target gene is operably associated with a tetracyline operator;
(c) developing a second protein expression profile for said derivative, wherein said derivative is grown is cultured in the presence of a subinhibitory level of tetracycline or a tetracycline analog, wherein said target gene is substantially underexpressed as compared to the expression of said target gene in the control strain; and
(d) comparing the first protein expression profile with the second protein expression profile to identify a protein expressed at a lower level in the second profile as compared to the level thereof in first profile.

30. An antibody that binds to a modified tetracycline repressor polypeptide of claim 16 or 17, wherein said antibody binds to said modified tetracycline repressor with an affinity greater than that with which said antibody binds to a wild-type tetracycline repressor polypeptide, wherein said wild-type tetracycline repressor polypeptide binds to a tetracycline operator sequence in a prokaryotic organism with a greater affinity in the absence of tetracycline or a tetracycline analogue than in the presence of tetracycline or the tetracycline analogue.

31. A method for producing a molecule selected from the group of proteins and nucleic acids, said method comprising:
a) providing a prokaryotic organism comprising a first expressible nucleic acid encoding a modified tetracycline repressor polypeptide of claim 16 or 17, and a second expressible nucleic acid encoding said molecule, wherein said second expressible nucleic acid is operably associated with a promoter and a tetracycline operator;
b) culturing said prokaryotic organism in a first growth medium comprising a first level of tetracycline or tetracycline analog for a first period of time sufficient to provide a plurality of said prokaryotic organisms; and
c) culturing said prokaryotic organism in a second growth medium comprising a second level of tetracycline or a tetracycline analog for a second period of time, wherein said second level of tetracycline or tetracycline analog is lower than said first level of tetracycline or tetracycline analog.

32. A method for modulating the level of synthesis of a target gene product in a prokaryotic cells, said method comprising:
a) providing a prokaryotic cell comprising a first expressible nucleic acid encoding a modified tetracycline repressor polypeptide according to claim 16 or 17, and a second expressible nucleic acid encoding an anti-RNA molecule, wherein said anti-RNA molecule inhibits synthesis of said target gene product, wherein said second expressible nucleic acid is operably associated with a promoter and a tetracycline operator; and
b) culturing said prokaryotic organism in a growth medium comprising a sub-inhibitory concentration tetracycline or a tetracycline analog for a period of time sufficient to provide a plurality of said prokaryotic organisms, whereby said level of synthesis is proportional to said concentration of tetracycline or tetracycline analog.

33. The isolated nucleic acid of claim 1, 2, or 3, wherein the encoded modified tetracycline repressor binds to a tetracycline operator sequence in a prokaryotic organism with a greater affinity in the presence of tetracycline or a tetracycline analog than in the absence of tetracycline or the tetracycline analog with a greater affinity at 28° C. than at 37° C.

34. The isolated nucleic acid of claim 1, 2, or 3, wherein the encoded modified tetracycline repressor binds to a tetracycline operator sequence in a prokaryotic organism with a greater affinity in the presence of tetracycline or a tetracycline analog than in the absence of tetracycline or the tetracycline analog with a greater affinity at 37° C. than at 28° C.

35. The method of claim 23, wherein the second expressible nucleic acid comprises the nucleic acid of claim 1, 2, or 3.
